(19) United States
(12) Patent Application Publication
(10) Pub. No.: US 2026/0076022 A1
(43) Pub. Date: Mar. 12, 2026

(54) COMPOSITION FOR AN ORGANIC OPTOELECTRONIC DEVICE, ORGANIC OPTOELECTRONIC DEVICE, AND DISPLAY DEVICE

(71) Applicant: SAMSUNG SDI CO., LTD., Yongin-si (KR)

(72) Inventors: Wook KIM, Suwon-si (KR); Dalho HUH, Suwon-si (KR); Hyung Sun KIM, Suwon-si (KR); Hanill LEE, Suwon-si (KR); Jonghoon KIM, Suwon-si (KR); Jiah YOON, Suwon-si (KR); Seonyeong GWAK, Suwon-si (KR); Suheon IM, Suwon-si (KR); Minji PARK, Suwon-si (KR); Jinseok JANG, Suwon-si (KR)

(21) Appl. No.: 19/321,489

(22) Filed: Sep. 8, 2025

(30) Foreign Application Priority Data
Sep. 11, 2024 (KR) 10-2024-0124263

(51) Int. Cl. H10K 50/11; H10K 85/40; H10K 85/60

(52) U.S. Cl. CPC H10K 50/11; H10K 85/40; H10K 85/622; H10K 85/626; H10K 85/633; H10K 85/636; H10K 85/654; H10K 85/6572; H10K 85/6574; H10K 85/6576

(57) ABSTRACT

A composition for an organic optoelectronic device, an organic optoelectronic device including the composition, and a display device including the organic optoelectronic device, the composition for an organic optoelectronic device including a first compound represented by Chemical Formula 1 and a second compound represented by a combination of Chemical Formula 2 and Chemical Formula 3:

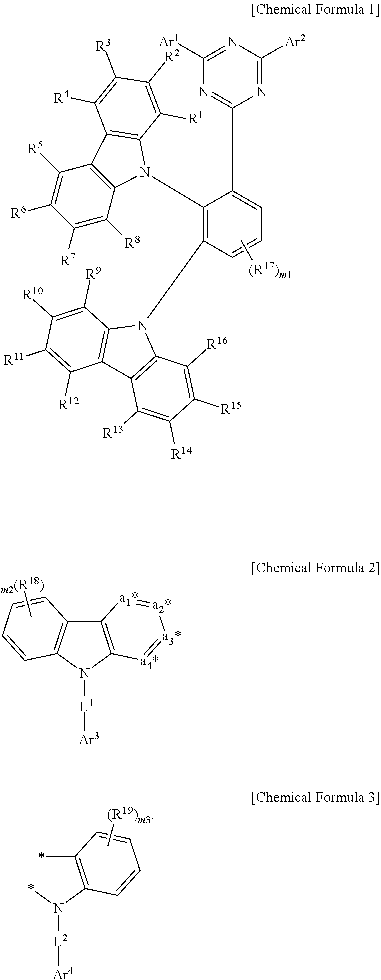

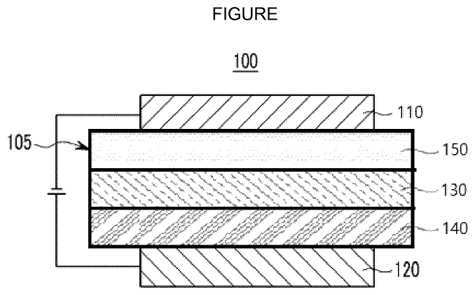

### CROSS-REFERENCE TO RELATED APPLICATION

This application claims priority to and the benefit of Korean Patent Application No. 10-2024-0124263 filed in the Korean Intellectual Property Office on Sep. 11, 2024, the entire contents of which are incorporated herein by reference.

### 1. FIELD

Embodiments relate to a composition for an organic optoelectronic device, an organic optoelectronic device, and a display device.

### 2. DESCRIPTION OF THE RELATED ART

An organic optoelectronic device (e.g., an organic optoelectronic diode) is a device capable of converting electrical energy and optical energy to each other.

Organic optoelectronic devices may be divided into two types according to a principle of operation. One is a photoelectric device that generates electrical energy by separating excitons formed by light energy into electrons and holes, and transferring the electrons and holes to different electrodes, respectively and the other is a light emitting device that generates light energy from electrical energy by supplying voltage or current to the electrodes.

Examples of the organic optoelectronic device may include an organic photoelectric device, an organic light emitting diode, an organic solar cell, or an organic photoconductor drum.

Among them, organic light emitting diodes (OLEDs) are attracting much attention in recent years due to increasing demands for flat panel display devices. The organic light emitting diode is a device that converts electrical energy into light, and the performance of the organic light emitting diode may be influenced by an organic material between electrodes.

### SUMMARY

The embodiments may be realized by providing a composition for an organic optoelectronic device, the composition including a first compound represented by Chemical Formula 1 and a second compound represented by a combination of Chemical Formula 2 and Chemical Formula 3:

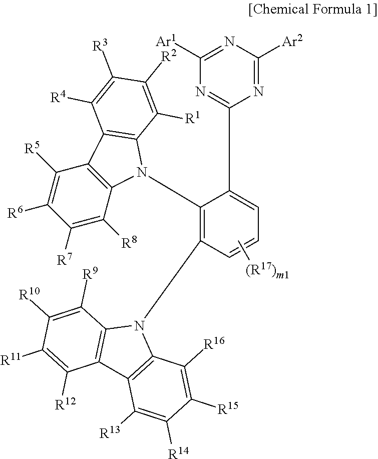

wherein, in Chemical Formula 1, R¹ to R¹⁷ are each independently hydrogen, deuterium, a cyano group, a substituted or unsubstituted C1 to C10 alkyl group, a substituted or unsubstituted C1 to C10 alkylsilyl group, or a substituted or unsubstituted C6 to C30 aryl group, at least one of R¹ to R¹⁶ is deuterium, a C1 to C10 alkyl group substituted with one or more deuterium atoms, a C1 to C10 alkylsilyl group substituted with one or more deuterium atoms, or a C6 to C30 aryl group substituted with one or more deuterium atoms, Ar¹ and Ar² are each independently a substituted or unsubstituted C6 to C30 aryl group or a substituted or unsubstituted C2 to C30 heterocyclic group, m1 is an integer of 1 to 3, and when m1 is 2 or 3, each R¹⁷ is the same or different from each other,

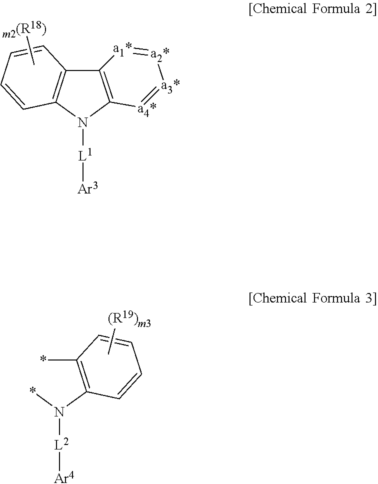

wherein, in Chemical Formula 2 and Chemical Formula 3, two adjacent ones of a₁* to a₄* of Chemical Formula 2, are linking carbons linked at * of Chemical Formula 3, the remaining two of a₁* to a₄* of Chemical Formula 2 that are not linked at * of Chemical Formula 3 are C-L^{a}-R^{a}, L^{a}, L¹, and L² are each independently a single bond or a substituted or unsubstituted C6 to C20 arylene group, R^{a}, R¹⁸, and R¹⁹ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group, Ar³ and Ar⁴ are each independently a substituted or unsubstituted C6 to C20 aryl group or a substituted or unsubstituted C2 to C30 heterocyclic group, m2 and m3 are each independently an integer of 1 to 4, when m2 is 2, 3, or 4, each R¹⁸ is the same or different from each other, and when m3 is 2, 3, or 4, each R¹⁹ is the same or different from each other.

The embodiments may be realized by providing an organic optoelectronic device including an anode and a cathode facing each other, and at least one organic layer between the anode and the cathode, wherein the organic layer includes the composition for an organic optoelectronic device according to some embodiments.

The embodiments may be realized by providing a display device including the organic optoelectronic device according to some embodiments.

### BRIEF DESCRIPTION OF THE DRAWING

Features will become apparent to those of skill in the art by describing in detail exemplary embodiments with reference to the attached drawings in which:

the FIG. is a cross-sectional view showing an organic light emitting diode according to some example embodiments.

### DETAILED DESCRIPTION

Example embodiments will now be described more fully hereinafter with reference to the accompanying drawings; however, they may be embodied in different forms and should not be construed as limited to the embodiments set forth herein. Rather, these embodiments are provided so that this disclosure will be thorough and complete, and will fully convey exemplary implementations to those skilled in the art.

In the drawing figures, the dimensions of layers and regions may be exaggerated for clarity of illustration. It will also be understood that when a layer or element is referred to as being “on” another layer or substrate, it can be directly on the other layer or substrate, or intervening layers may also be present. Further, it will be understood that when a layer is referred to as being “under” another layer, it can be directly under, and one or more intervening layers may also be present. In addition, it will also be understood that when a layer is referred to as being “between” two layers, it can be the only layer between the two layers, or one or more intervening layers may also be present. Like reference numerals refer to like elements throughout.

As used herein, when a definition is not otherwise provided, “substituted” refers to replacement of at least one hydrogen of a substituent or a compound by deuterium, a halogen, a hydroxyl group, an amino group, a substituted or unsubstituted C1 to C30 amine group, a nitro group, a substituted or unsubstituted C1 to C40 silyl group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C20 alkoxy group, a C1 to C10 trifluoroalkyl group, a cyano group, or a combination thereof.

In one example, the “substituted” refers to replacement of at least one hydrogen of a substituent or a compound by deuterium, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, or a cyano group. In a specific example, the “substituted” refers to replacement of at least one hydrogen of a substituent or a compound by deuterium, a C1 to C20 alkyl group, a C1 to C5 alkylsilyl group, a C6 to C20 aryl group, a C2 to C20 heteroaryl group, or a cyano group. In a specific example, the “substituted” refers to replacement of at least one hydrogen of a substituent or a compound by deuterium, a C1 to C5 alkyl group, a C1 to C5 alkylsilyl group, a C6 to C18 aryl group, a C2 to C18 heteroaryl group, or a cyano group. In a specific example, the “substituted” refers to replacement of at least one hydrogen of a substituent or a compound by deuterium, a cyano group, a methyl group, an ethyl group, an n-propyl group, an isopropyl group, an n-butyl group, an iso-butyl group, a sec-butyl group, a tert-butyl group, a trimethylsilyl group, a phenyl group, a biphenyl group, a terphenyl group, or a naphthyl group.

“Unsubstituted” refers to non-replacement of a hydrogen atom by another substituent and remaining of the hydrogen atom.

In the present specification, “hydrogen substitution (—H)” may include “deuterium substitution (-D)” or “tritium substitution (-T)”.

As used herein, when a definition is not otherwise provided, “hetero” refers to one including one to three heteroatoms selected from N, O, S, P, and Si, and remaining carbons in one functional group.

As used herein, “aryl group” refers to a group including at least one hydrocarbon aromatic moiety, and all elements of the hydrocarbon aromatic moiety have p-orbitals which form conjugation, for example a phenyl group, a naphthyl group, or the like, two or more hydrocarbon aromatic moieties may be linked by a sigma bond and may be, for example a biphenyl group, a terphenyl group, a quarterphenyl group, or the like, and two or more hydrocarbon aromatic moieties may be fused directly or indirectly to provide a non-aromatic fused ring, for example a fluorenyl group.

The aryl group may include a monocyclic, polycyclic, or fused ring polycyclic (i.e., rings sharing adjacent pairs of carbon atoms) functional group.

As used herein, “heterocyclic group” is a generic concept of a heteroaryl group, and may include at least one heteroatom selected from N, O, S, P, and Si instead of carbon (C) in a cyclic compound such as aryl group, a cycloalkyl group, a fused ring thereof, or a combination thereof. When the heterocyclic group is a fused ring, the entire ring or each ring of the heterocyclic group may include one or more heteroatoms.

In an implementation, “heteroaryl group” may refer to aryl group including at least one heteroatom selected from N, O, S, P, and Si. Two or more heteroaryl groups are linked by a sigma bond directly, or when the heteroaryl group includes two or more rings, the two or more rings may be fused. When the heteroaryl group is a fused ring, each ring may include one to three heteroatoms.

More specifically, the substituted or unsubstituted C6 to C30 aryl group may be a substituted or unsubstituted phenyl group, a substituted or unsubstituted naphthyl group, a substituted or unsubstituted anthracenyl group, a substituted or unsubstituted phenanthrenyl group, a substituted or unsubstituted naphthacenyl group, a substituted or unsubstituted pyrenyl group, a substituted or unsubstituted biphenyl group, a substituted or unsubstituted p-terphenyl group, a substituted or unsubstituted m-terphenyl group, a substituted or unsubstituted o-terphenyl group, a substituted or unsubstituted chrysenyl group, a substituted or unsubstituted benzophenanthrenyl group, a substituted or unsubstituted triphenylene group, a substituted or unsubstituted perylenyl group, a substituted or unsubstituted fluorenyl group, a substituted or unsubstituted indenyl group, or a combination thereof.

More specifically, the substituted or unsubstituted C2 to C30 heterocyclic group may be a substituted or unsubstituted furanyl group, a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzthiazinyl group, a substituted or unsubstituted acridinyl group, a substituted or unsubstituted phenazinyl group, a substituted or unsubstituted phenothiazinyl group, a substituted or unsubstituted phenoxazinyl group, a substituted or unsubstituted carbazolyl group, a substituted or unsubstituted dibenzofuranyl group, a substituted or unsubstituted dibenzothiophenyl group, a substituted or unsubstituted benzonaphthofuranyl group, a substituted or unsubstituted benzonaphthothiophenyl group, a substituted or unsubstituted benzofuranofluorenyl group, a substituted or unsubstituted benzothiophenefluorenyl group, or a combination thereof.

As used herein, hole characteristics refer to an ability to donate an electron to form a hole when an electric field is applied and that a hole formed in the anode may be easily injected into the light emitting layer and transported in the light emitting layer due to conductive characteristics according to a highest occupied molecular orbital (HOMO) level.

In addition, electron characteristics refer to an ability to accept an electron when an electric field is applied and that electron formed in the cathode may be easily injected into the light emitting layer and transported in the light emitting layer due to conductive characteristics according to a lowest unoccupied molecular orbital (LUMO) level.

Hereinafter, a composition for an organic optoelectronic device according to some example embodiments is described.

A composition for an organic optoelectronic device according to some example embodiments includes a first compound and a second compound, wherein the first compound may be represented by Chemical Formula 1.

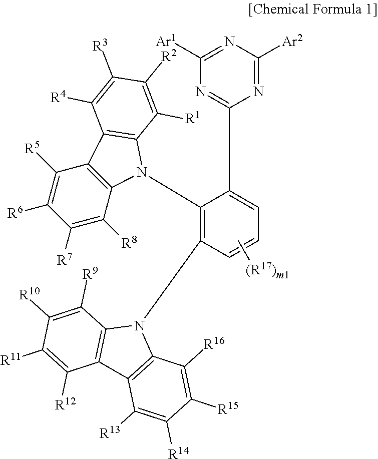

In Chemical Formula 1, R¹ to R¹⁷ may each independently be or include, e.g., hydrogen, deuterium, a cyano group, a substituted or unsubstituted C1 to C10 alkyl group, a substituted or unsubstituted C1 to C10 alkylsilyl group, or a substituted or unsubstituted C6 to C30 aryl group.

At least one of R¹ to R¹⁶ may be or include, e.g., deuterium, a C1 to C10 alkyl group substituted with one or more deuterium atoms, a C1 to C10 alkylsilyl group substituted with one or more deuterium atoms, or a C6 to C30 aryl group substituted with one or more deuterium atoms.

Ar¹ and Ar² may each independently be or include, e.g., a substituted or unsubstituted C6 to C30 aryl group or a substituted or unsubstituted C2 to C30 heterocyclic group.

m1 may be, e.g., an integer of 1 to 3.

In an implementation, m1 may be 2 or 3, and each R¹⁷ may be the same or different from each other.

The compound represented by Chemical Formula 1 has a structure in which carbazoles are substituted at the ortho and meta positions with respect to a triazine, e.g., the carbazole groups are substituted at ortho and meta positions of a phenyl group bonded to the triazine group, and carbazoles are additionally substituted with deuterium, thereby increasing the stability of a moiety having hole characteristics for electrons, and an organic light emitting diode including the compound can realize significantly improved life-span characteristics while maintaining high efficiency.

In an implementation, in Chemical Formula 1, at least one of R¹ to R⁸ may be, e.g., deuterium, a C1 to C10 alkyl group substituted with one or more deuterium atoms, a C1 to C10 alkylsilyl group substituted with one or more deuterium atoms, or a C6 to C30 aryl group substituted with one or more deuterium atoms.

In an implementation, in Chemical Formula 1, at least one of R⁹ to R¹⁶ may be, e.g., deuterium, a C1 to C10 alkyl group substituted with one or more deuterium atoms, a C1 to C10 alkylsilyl group substituted with one or more deuterium atoms, or a C6 to C30 aryl group substituted with one or more deuterium atoms.

In an implementation, in Chemical Formula 1, R¹ to R⁸ may each independently be, e.g., deuterium, a C1 to C10 alkyl group substituted with one or more deuterium atoms, a C1 to C10 alkylsilyl group substituted with one or more deuterium atoms, or a C6 to C30 aryl group substituted with one or more deuterium atoms.

In an implementation, in Chemical Formula 1, R⁹ to R¹⁶ may each independently be, e.g., deuterium, a C1 to C10 alkyl group substituted with one or more deuterium atoms, a C1 to C10 alkylsilyl group substituted with one or more deuterium atoms, or a C6 to C30 aryl group substituted with one or more deuterium atoms.

In an implementation, R¹ to R¹⁶ may each independently be, e.g., deuterium, a C1 to C10 alkyl group substituted with one or more deuterium atoms, a C1 to C10 alkylsilyl group substituted with one or more deuterium atoms, or a C6 to C30 aryl group substituted with one or more deuterium atoms.

In an implementation, each of R¹ to R¹⁶ may be deuterium.

In an implementation, Ar¹ and Ar² may each independently be, e.g., a substituted or unsubstituted phenyl group, a substituted or unsubstituted biphenyl group, a substituted or unsubstituted dibenzofuranyl group, a substituted or unsubstituted dibenzothiophenyl group, or a substituted or unsubstituted carbazolyl group.

In an implementation, R¹⁷ may be, e.g., hydrogen, deuterium, a substituted or unsubstituted C1 to C6 alkyl group, a substituted or unsubstituted C1 to C6 alkylsilyl group, or a substituted or unsubstituted C6 to C12 aryl group.

In an implementation, R¹⁷ may be, e.g., hydrogen, deuterium, a substituted or unsubstituted phenyl group.

In an implementation, the first compound represented by Chemical Formula 1 may be, e.g., a compound of Group 1.

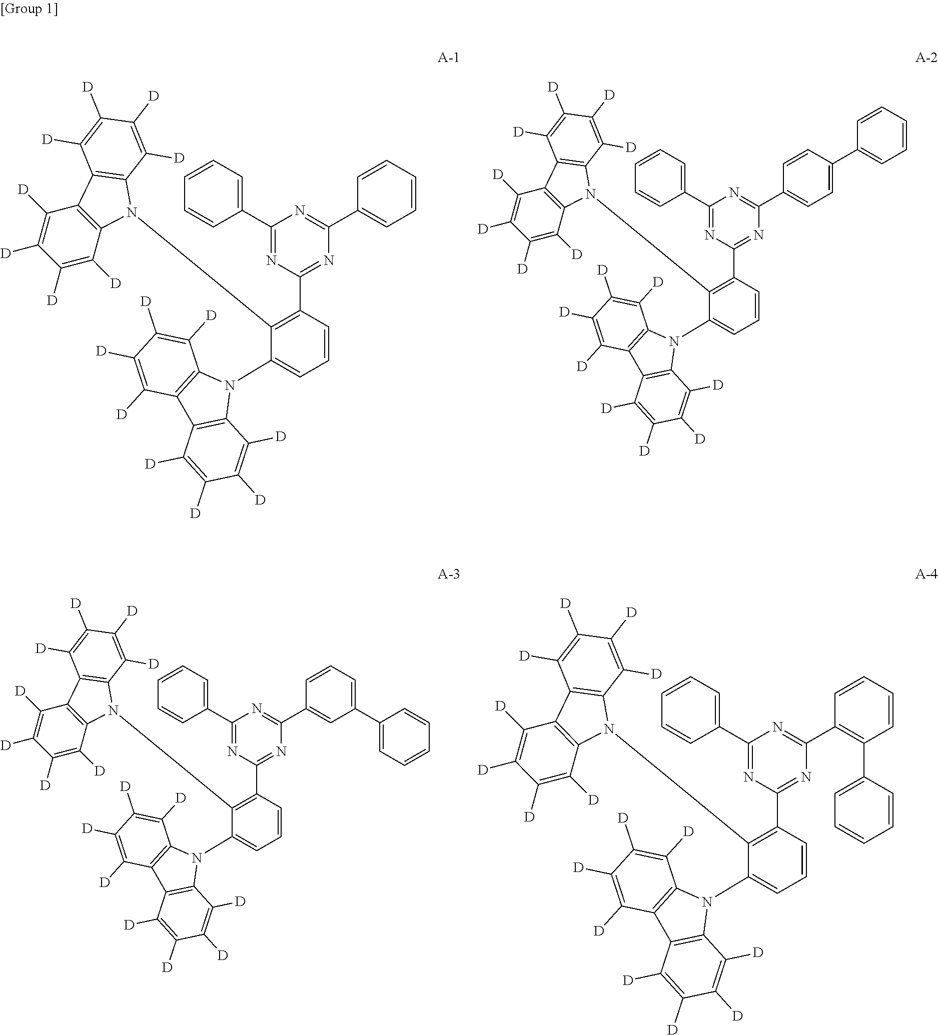

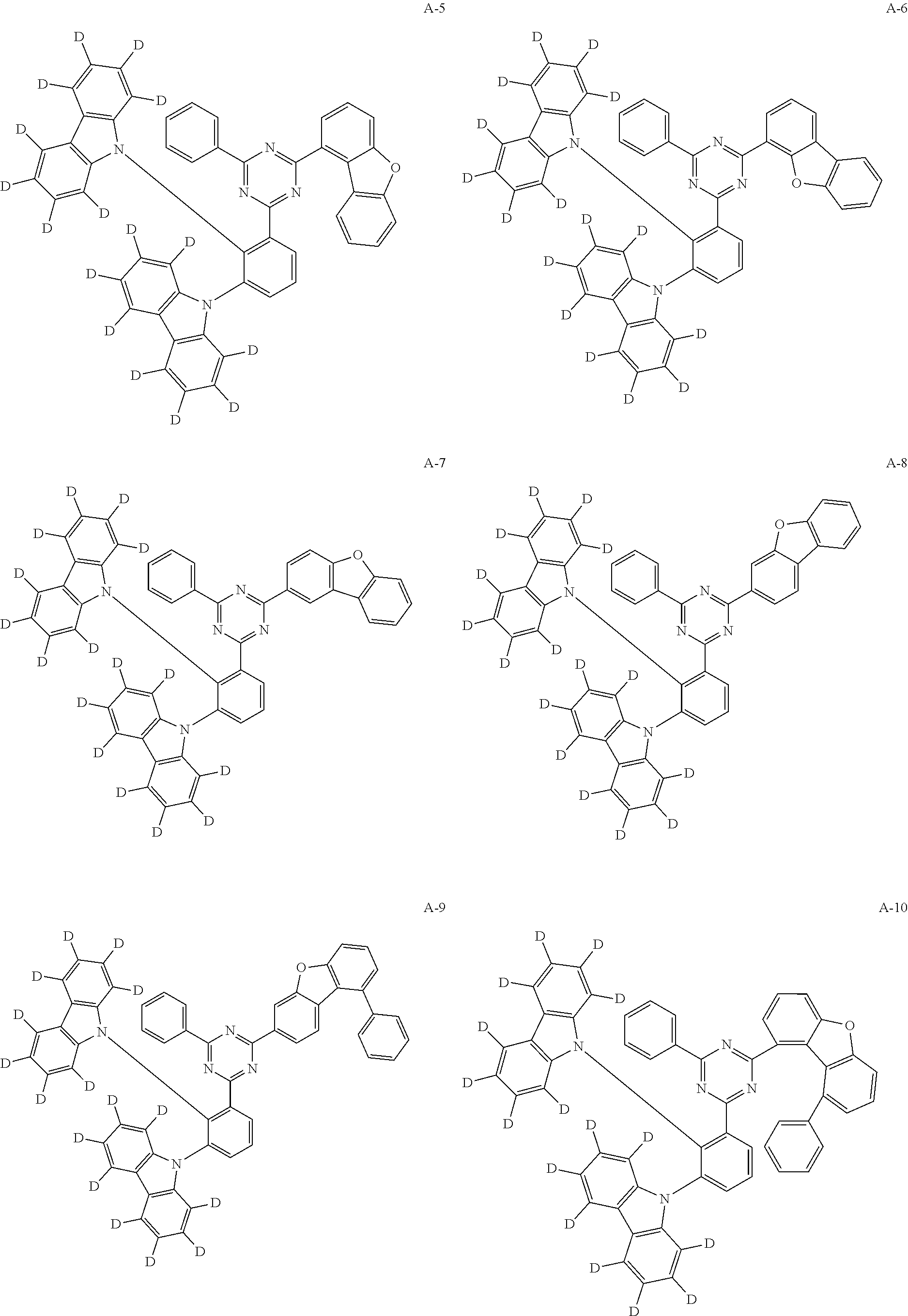

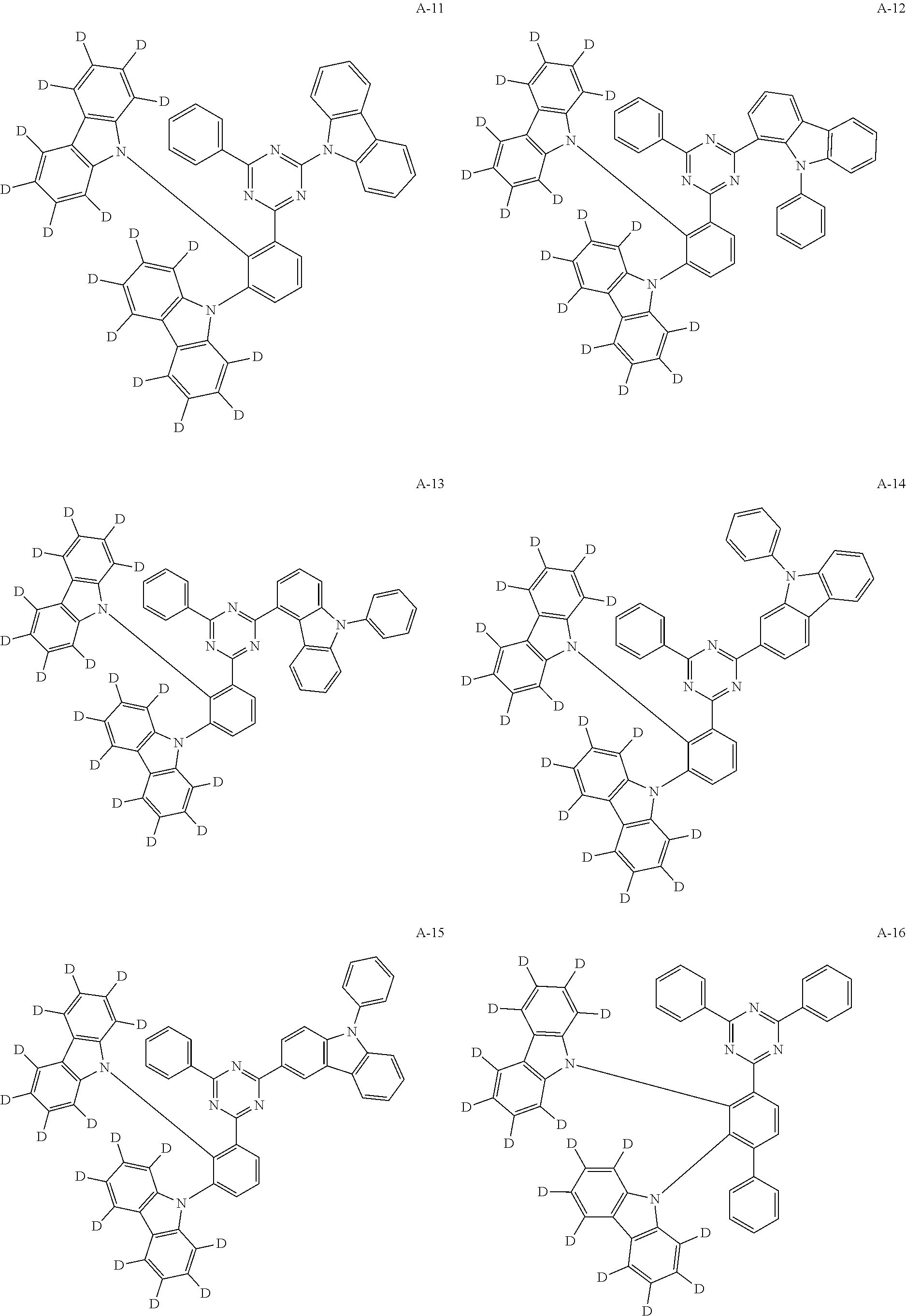

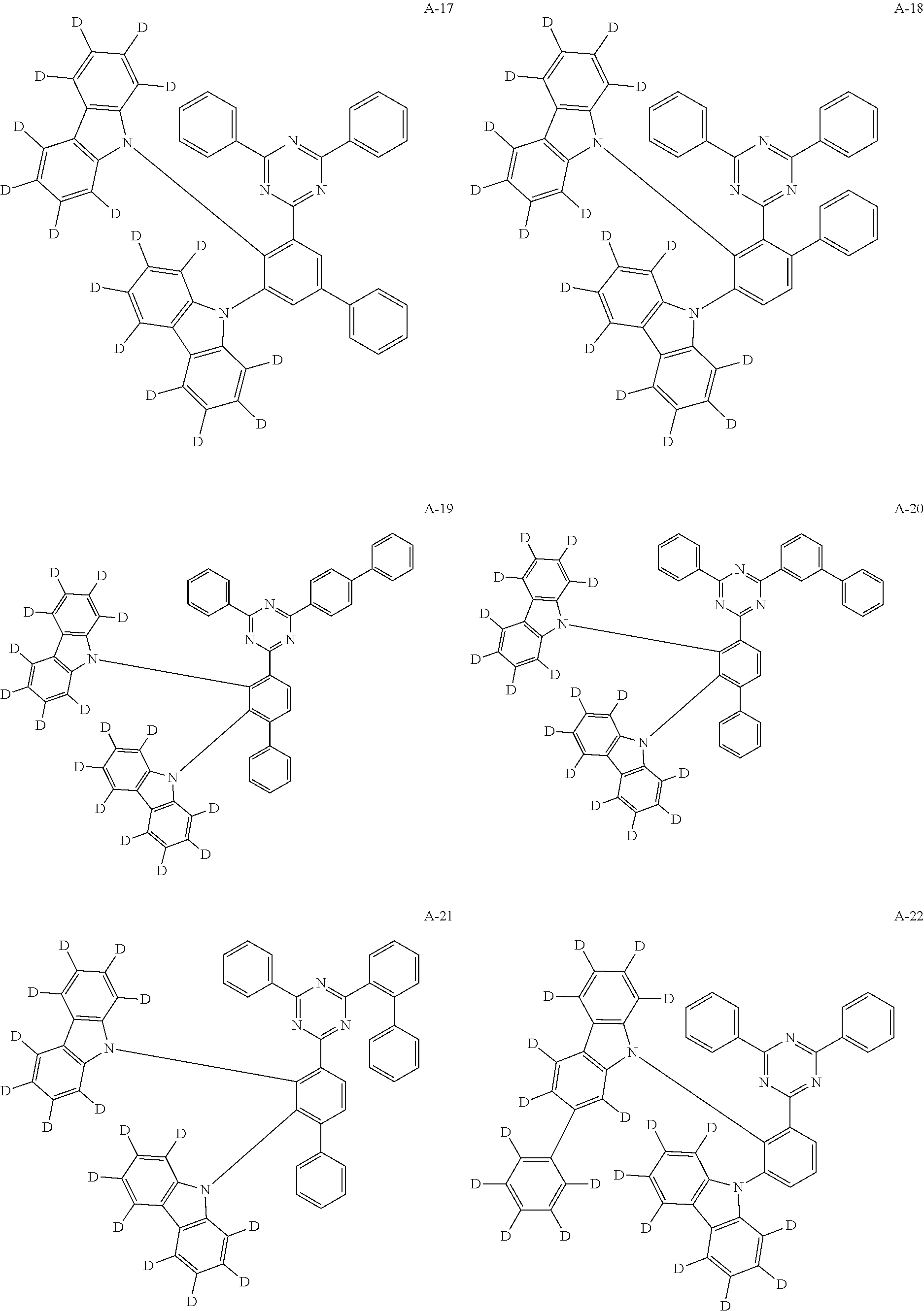

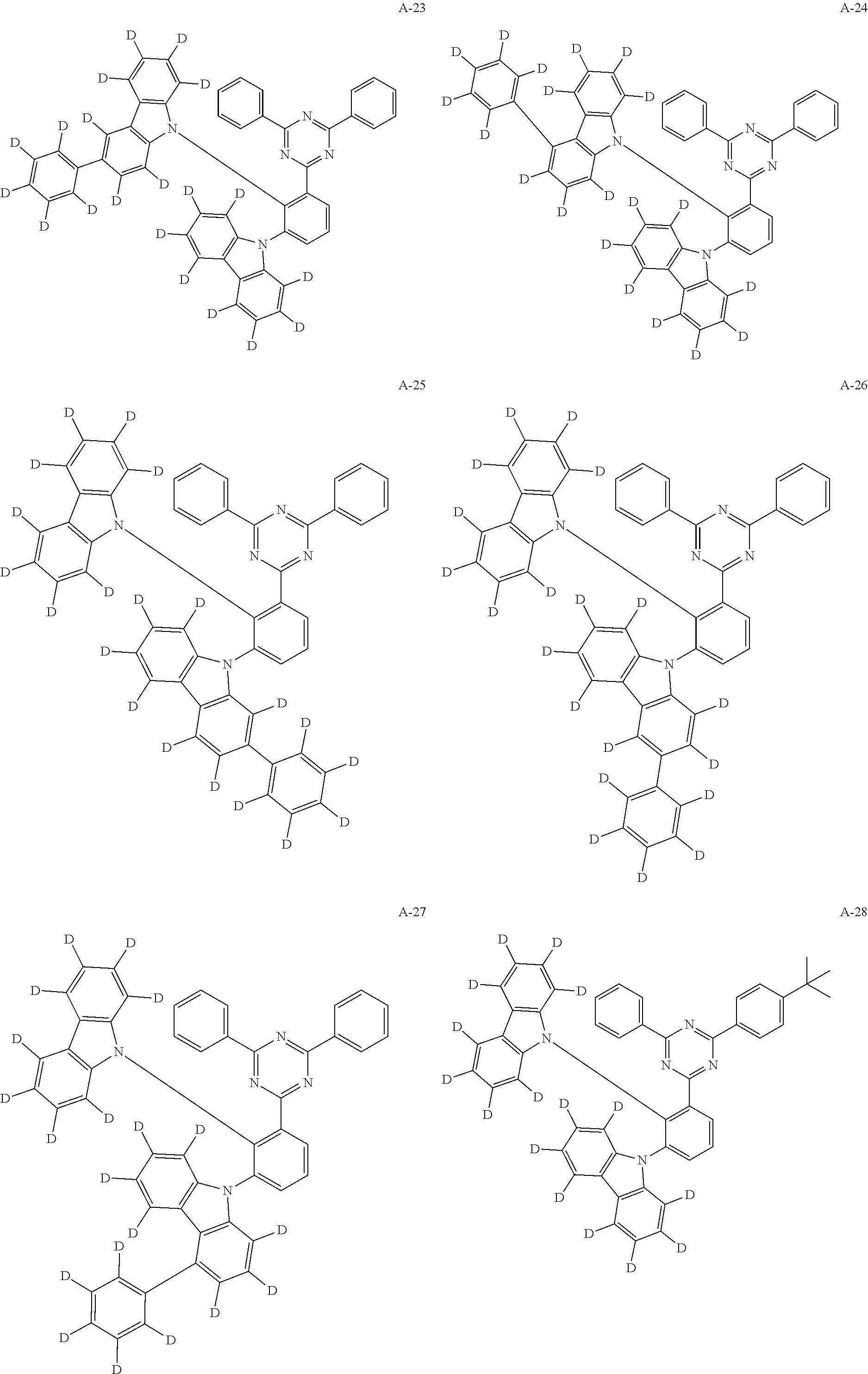

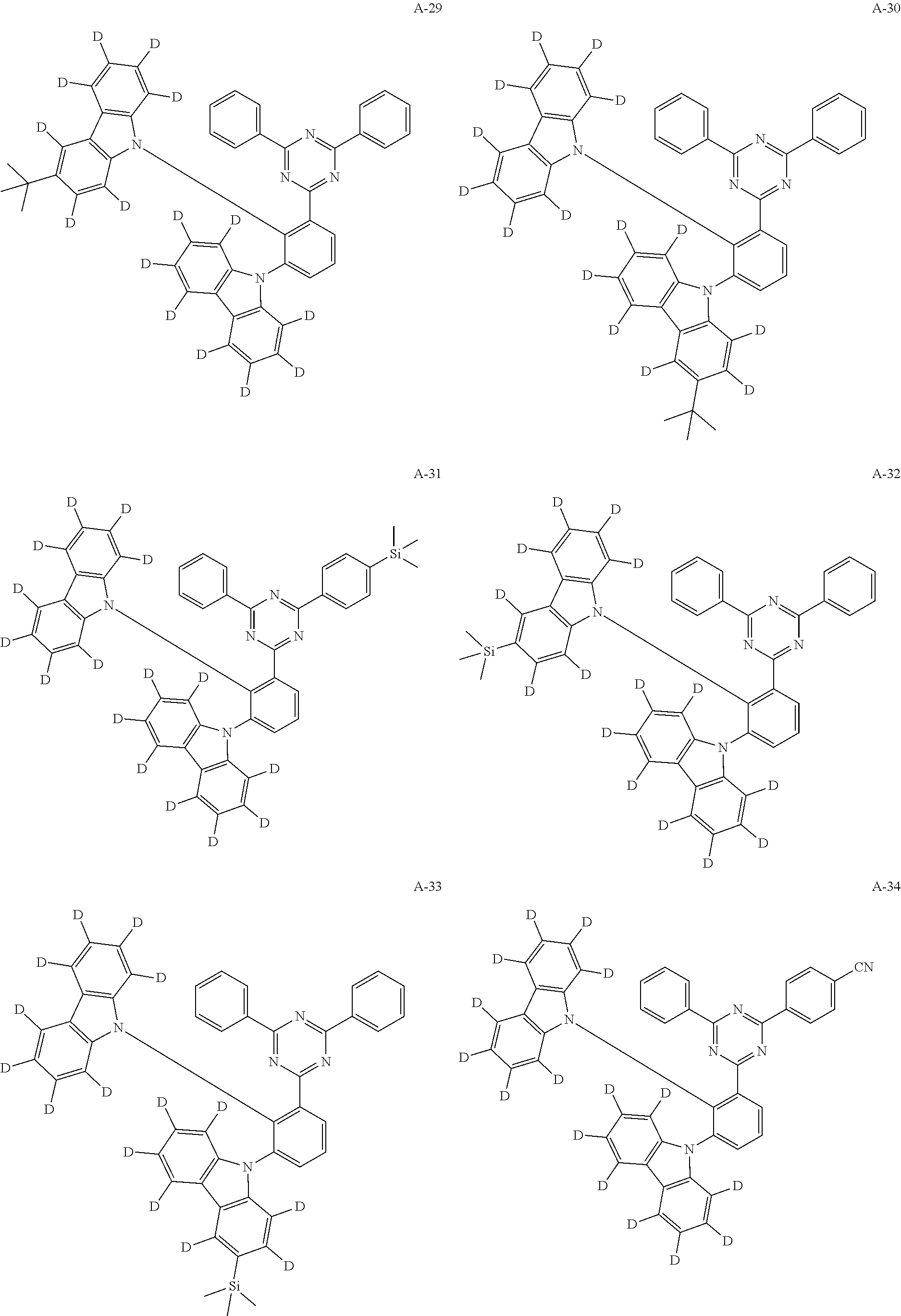

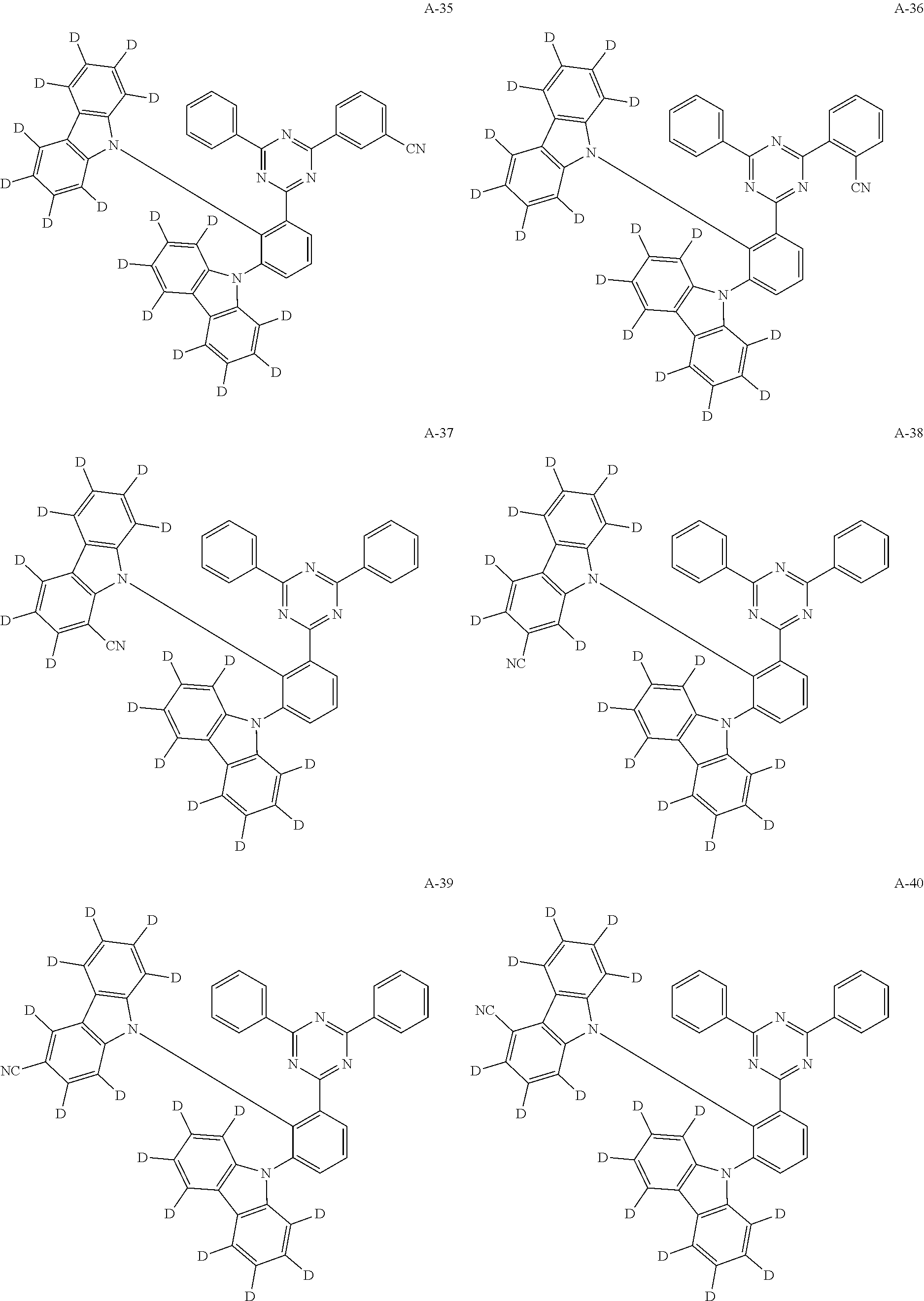

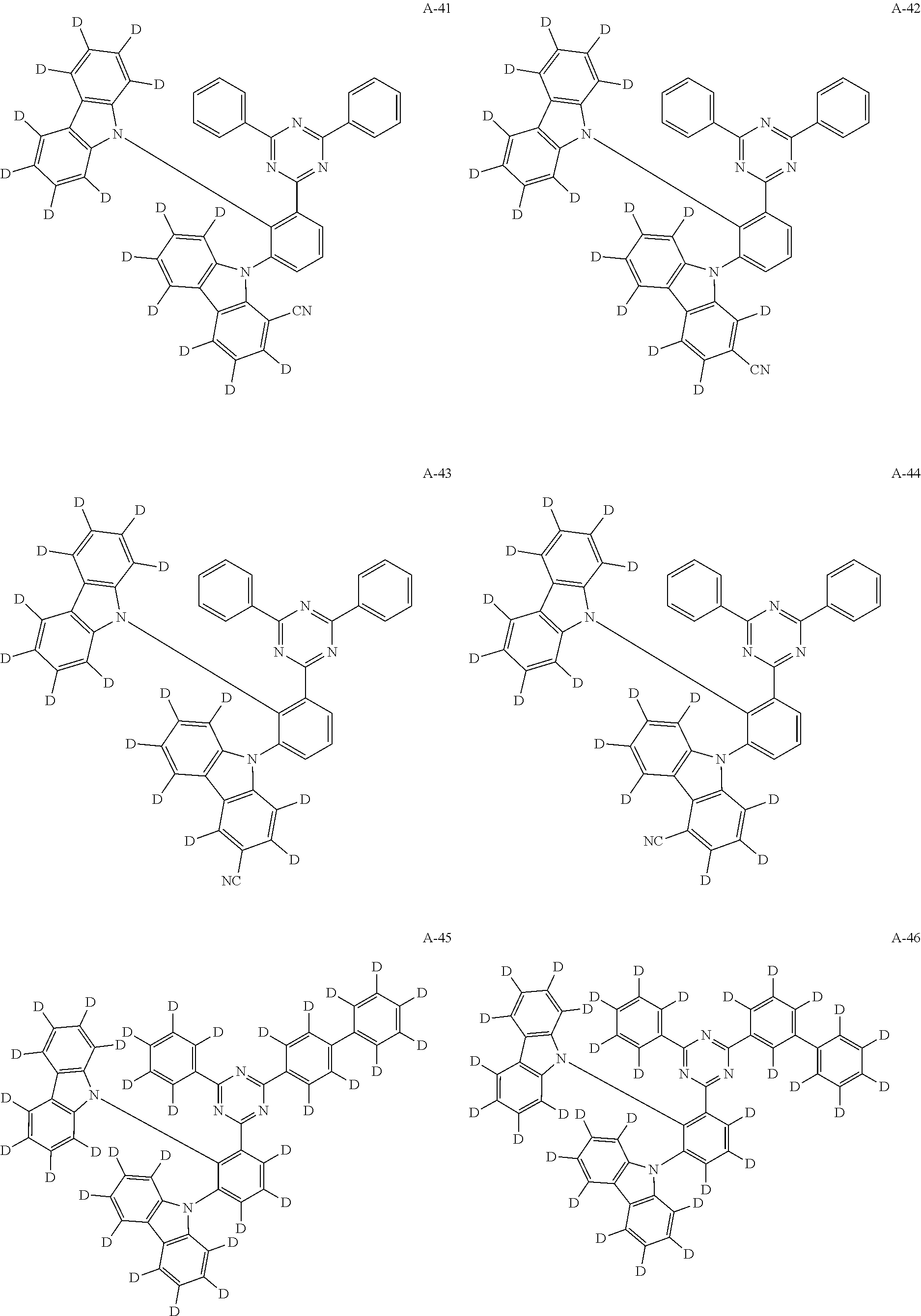

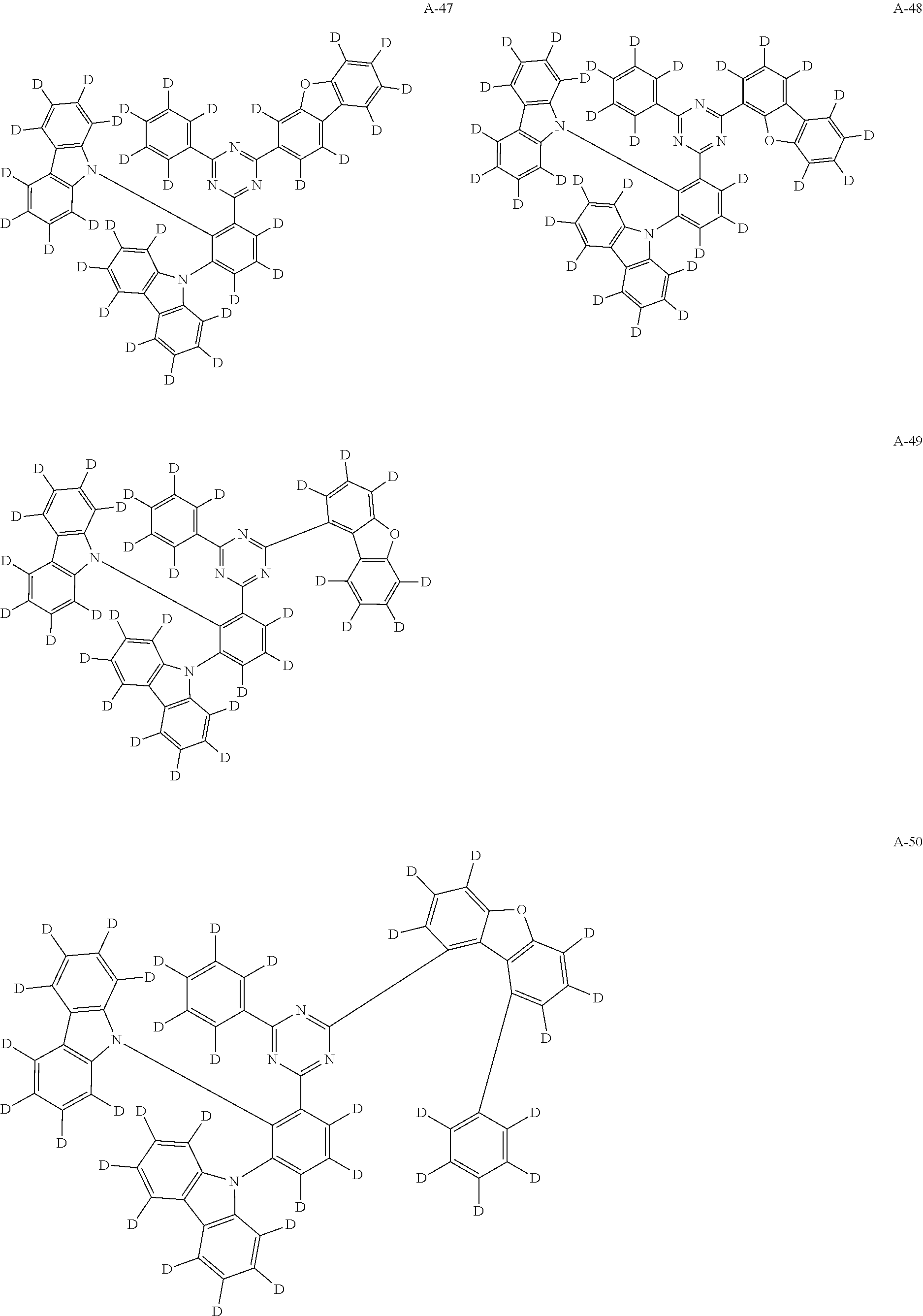

The second compound may be represented by a combination of Chemical Formula 2 and Chemical Formula 3.

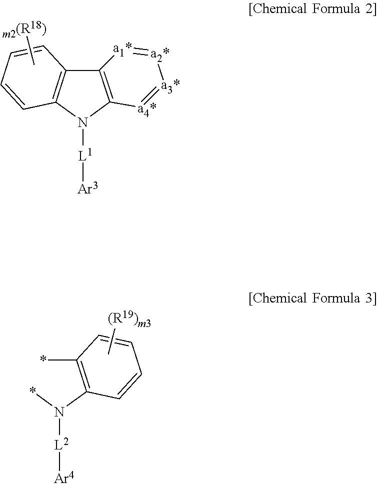

In Chemical Formula 2 and Chemical Formula 3, two adjacent ones of a₁* to a₄* of Chemical Formula 2, may be linking carbons linked at * of Chemical Formula 3.

The remaining two of a₁* to a₄* of Chemical Formula 2 that are not linked at * of Chemical Formula 3 may be, e.g., C-L^{a}-R^{a}.

L^{a}, L¹, and L² may each independently be, e.g., a single bond or a substituted or unsubstituted C6 to C20 arylene group.

R^{a}, R¹⁸, and R¹⁹ may each independently be, e.g., hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group.

Ar³ and Ar⁴ may each independently be, e.g., a substituted or unsubstituted C6 to C20 aryl group or a substituted or unsubstituted C2 to C30 heterocyclic group.

m2 and m3 may each independently be, e.g., an integer of 1 to 4.

In an implementation, m2 may be, e.g., 2, 3, or 4, and each R¹⁹ may be the same or different from each other.

In an implementation, m3 may be, e.g., 2, 3, or 4, and each R¹¹ may be the same or different from each other.

The second compound may be used in the light emitting layer together with the first compound to improve luminous efficiency and life-span characteristics by increasing charge mobility and stability.

In an implementation, in Chemical Formula 2 and Chemical Formula 3, “substituted” may refer to replacement of at least one hydrogen by deuterium, a C1 to C₄ alkyl group, a C6 to C18 aryl group, or a C2 to C30 heteroaryl group.

The combination of Chemical Formula 2 and Chemical Formula 3 may be represented, e.g., by one of Chemical Formula 2A, Chemical Formula 2B, Chemical Formula 2C, Chemical Formula 2D, or Chemical Formula 2E.

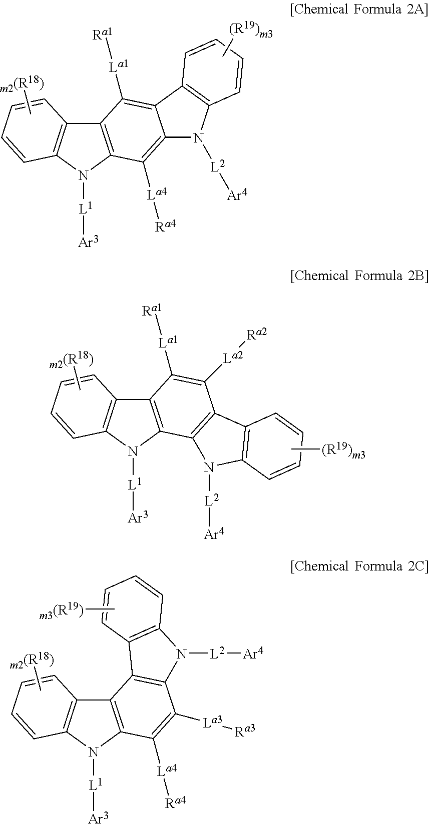

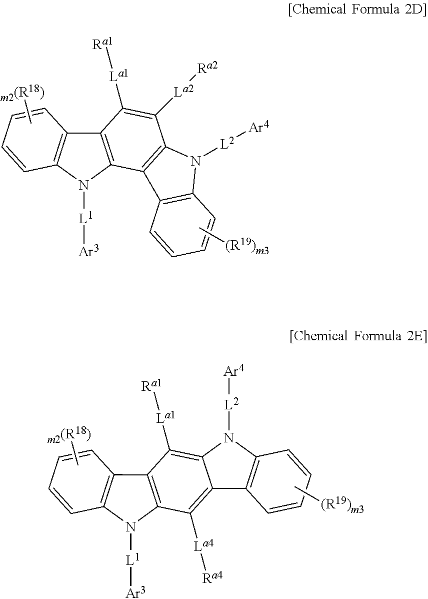

In Chemical Formula 2A to Chemical Formula 2E, L, L², Ar³, Ar⁴, R¹⁸, R¹⁹, m2, and m3 may be defined the same as described above.

L^{a1} to L^{a4} may be defined the same as L¹ and L² described above.

R^{a1} to R^{a4} may be defined the same as R¹⁸ and R¹⁹ described above.

In an implementation, in Chemical Formula 2 and Chemical Formula 3, Ar³ and Ar⁴ may each independently be, e.g., a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted triphenylenyl group, a substituted or unsubstituted carbazolyl group, a substituted or unsubstituted dibenzothiophenyl group, a substituted or unsubstituted dibenzofuranyl group, or a substituted or unsubstituted fluorenyl group.

R^{a1} to R^{a4}, R¹⁸, and R¹⁹ may each independently be, e.g., hydrogen, deuterium, a cyano group, a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

In an implementation, in Chemical Formulas 2 and 3, moieties *-L-Ar³ and *-L²-Ar⁴ may each independently be, e.g., a moiety of Group I.

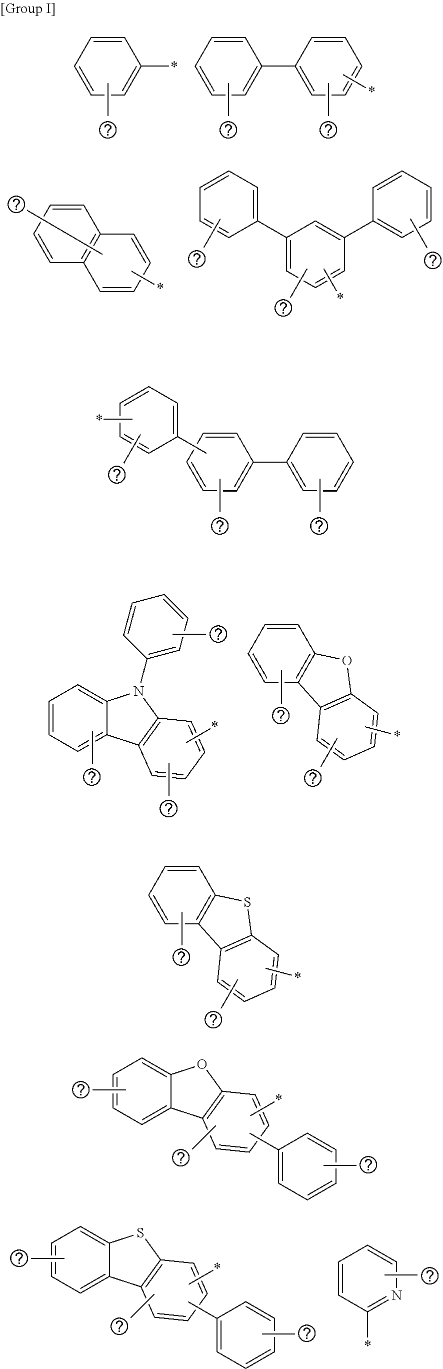

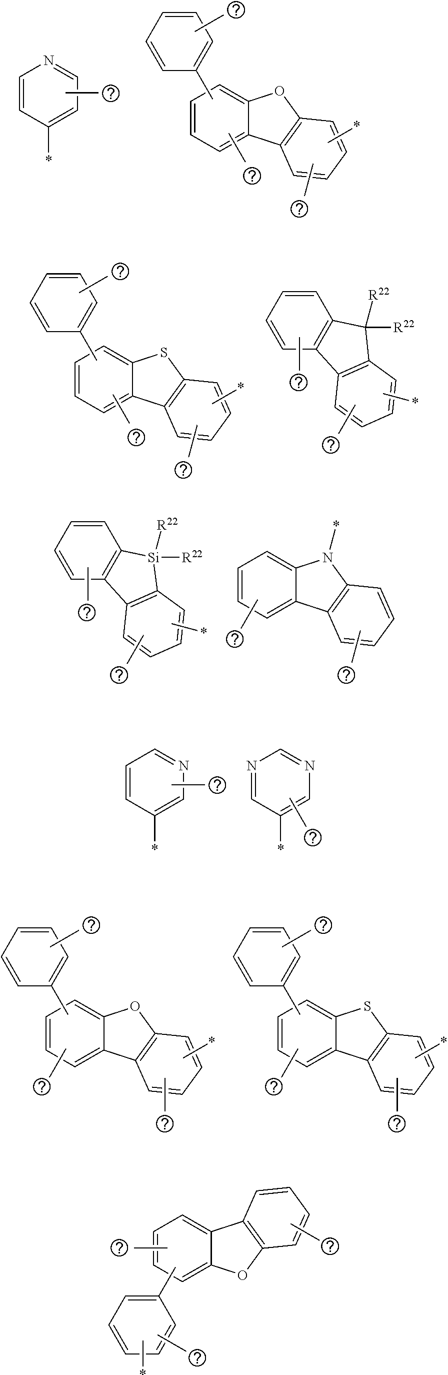

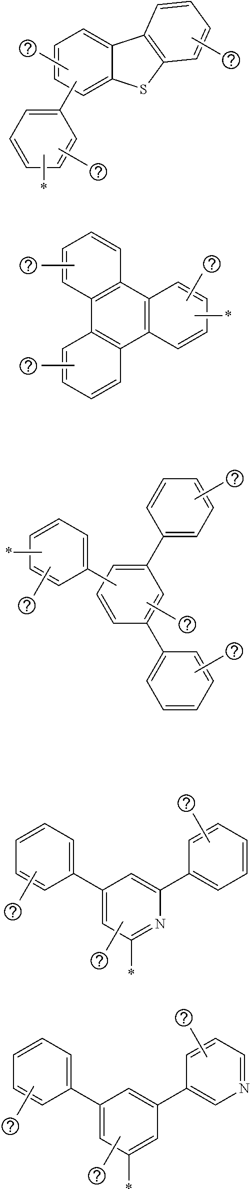

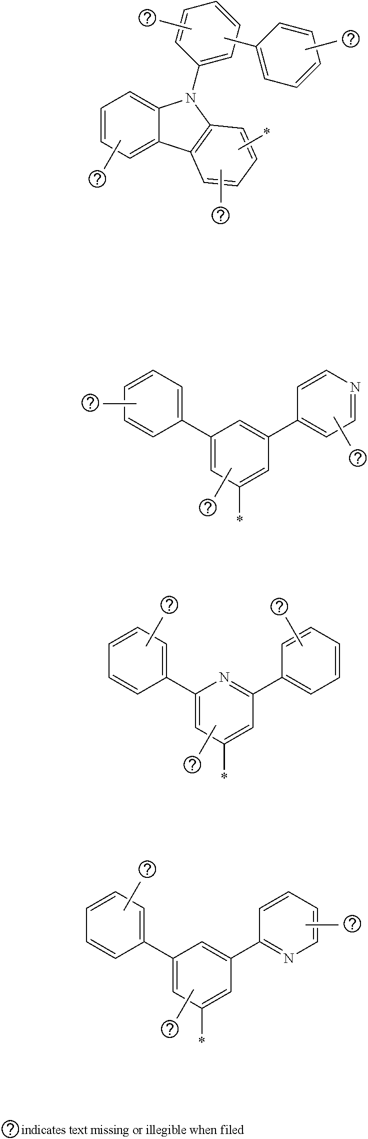

In Group I, R²⁰ to R²⁴ may each independently be, e.g., hydrogen, deuterium, a cyano group, a substituted or unsubstituted C1 to C10 alkyl group, or a substituted or unsubstituted C6 to C12 aryl group.

m4 may be, e.g., an integer of 1 to 5.

m5 may be, e.g., an integer of 1 to 4.

m6 may be, e.g., an integer of 1 to 3.

m7 may be, e.g., an integer of 1 or 2.

m8 may be, e.g., an integer of 1 to 7.

* is a linking point.

In an implementation, m4 may be 2, 3, 4, or 5, and each R²⁰ may be the same or different from each other.

In an implementation, m5 may be 2, 3, or 4, and each R²¹ may be the same or different from each other.

In an implementation, m6 may be 2 or 3, and each R²² may be the same or different from each other.

In an implementation, when m7 may be 2, and each R²³ may be the same or different from each other.

In an implementation, when m8 may be 2, 3, 4, 5, 6, or 7, and each R²⁴ may be the same or different from each other.

In an implementation, R^{a1} to R^{a4}, R¹⁸, and R¹⁹ may each independently be, e.g., hydrogen, deuterium, a cyano group, a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

In an implementation, R^{a1} to R^{a4}, R¹⁸, and R¹⁹ may each independently be, e.g., hydrogen, deuterium, a cyano group, or a substituted or unsubstituted phenyl group.

In an implementation, R^{a1} to R^{a4}, R¹⁸, and R¹⁹ may each independently be, e.g., hydrogen, deuterium, or a substituted or unsubstituted phenyl group.

In an implementation, the second compound may be represented by Chemical Formula 2C, wherein in Chemical Formula 2C, L^{a3} and L^{a4} may each be a single bond, L¹ and L² may each independently be, e.g., a single bond or a substituted or unsubstituted C6 to C12 arylene group, R¹⁸, R¹⁹, R^{a3}, and R^{a4} may each independently be, e.g., hydrogen, deuterium or a substituted or unsubstituted phenyl group, and Ar³ and Ar⁴ may each independently be, e.g., a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

In an implementation, in Chemical Formula 2C, L^{a3} and L^{a4} may each be a single bond, R¹⁸, R¹⁹, R^{a3}, and R^{a4} may each independently be, e.g., hydrogen, deuterium, or a substituted or unsubstituted C6 to C12 aryl group, and moieties *-L¹-Ar³ and *-L²-Ar⁴ may each independently be, e.g., a moiety of Group I.

In an implementation, the second compound may be, e.g., a compound of Group 2.

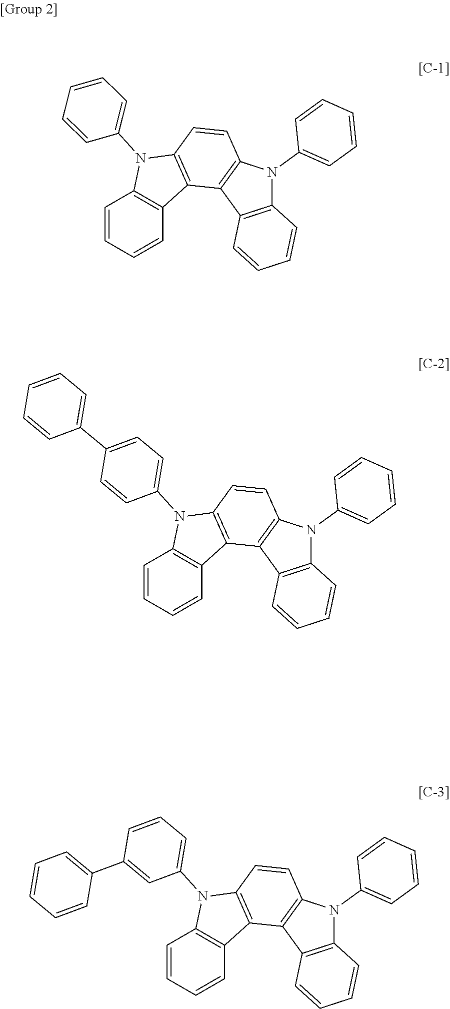 [SMILES: c1ccc(-n2c3ccccc3c3c4c5ccccc5n(-c5ccccc5)c4ccc32)cc1 ; c1ccc(-c2ccc(-n3c4ccccc4c4c5c6ccccc6n(-c6ccccc6)c5ccc43)cc2)cc1 ; c1ccc(-c2cccc(-n3c4ccccc4c4c5c6ccccc6n(-c6ccccc6)c5ccc43)c2)cc1]

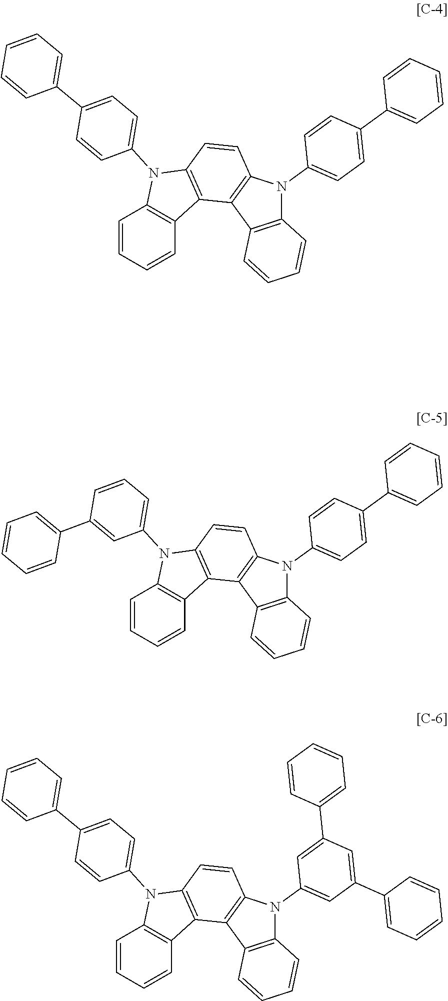 [SMILES: c1ccc(-c2ccc(-n3c4ccccc4c4c5c6ccccc6n(-c6ccc(-c7ccccc7)cc6)c5ccc43)cc2)cc1 ; c1ccc(-c2ccc(-n3c4ccccc4c4c5c6ccccc6n(-c6cccc(-c7ccccc7)c6)c5ccc43)cc2)cc1 ; c1ccc(-c2ccc(-n3c4ccccc4c4c5c6ccccc6n(-c6cc(-c7ccccc7)cc(-c7ccccc7)c6)c5ccc43)cc2)cc1]

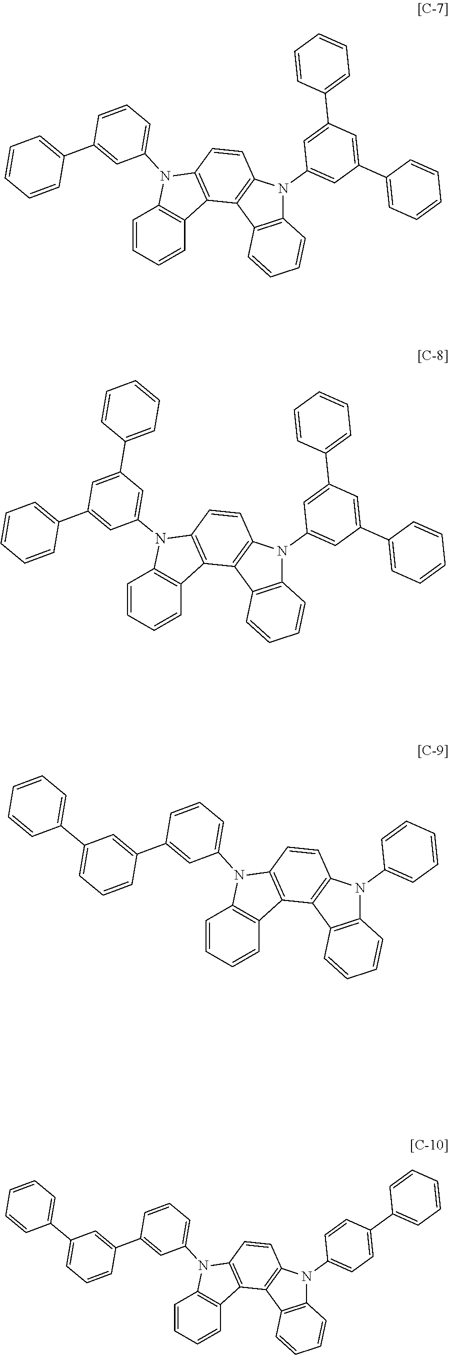 [SMILES: c1ccc(-c2cccc(-n3c4ccccc4c4c5c6ccccc6n(-c6cc(-c7ccccc7)cc(-c7ccccc7)c6)c5ccc43)c2)cc1 ; c1ccc(-c2cc(-c3ccccc3)cc(-n3c4ccccc4c4c5c6ccccc6n(-c6cc(-c7ccccc7)cc(-c7ccccc7)c6)c5ccc43)c2)cc1 ; c1ccc(-c2cccc(-c3cccc(-n4c5ccccc5c5c6c7ccccc7n(-c7ccccc7)c6ccc54)c3)c2)cc1 ; c1ccc(-c2ccc(-n3c4ccccc4c4c5c6ccccc6n(-c6cccc(-c7cccc(-c8ccccc8)c7)c6)c5ccc43)cc2)cc1]

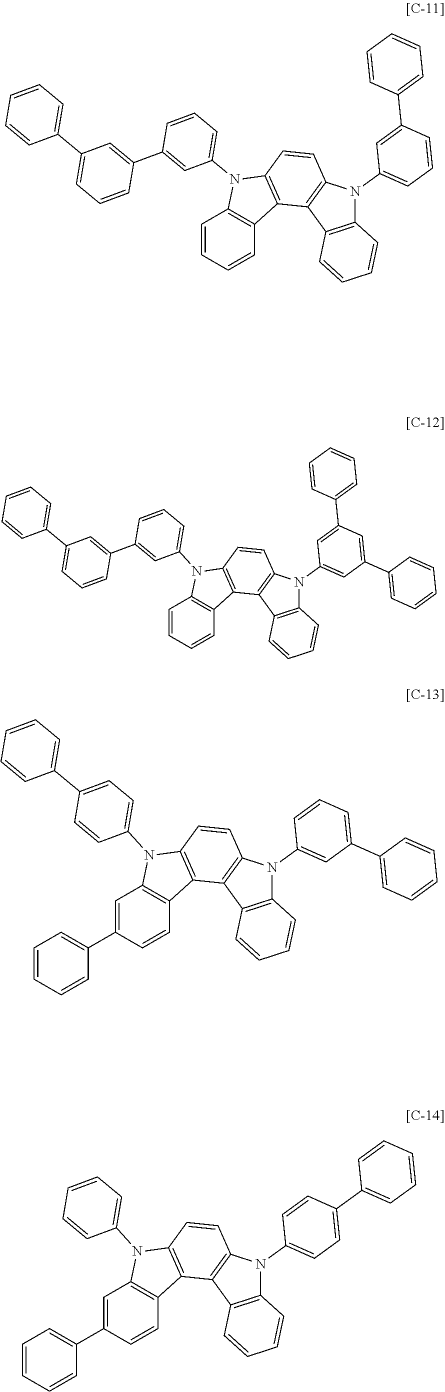 [SMILES: c1ccc(-c2cccc(-c3cccc(-n4c5ccccc5c5c6c7ccccc7n(-c7cccc(-c8ccccc8)c7)c6ccc54)c3)c2)cc1 ; c1ccc(-c2cccc(-c3cccc(-n4c5ccccc5c5c6c7ccccc7n(-c7cc(-c8ccccc8)cc(-c8ccccc8)c7)c6ccc54)c3)c2)cc1 ; c1ccc(-c2ccc(-n3c4cc(-c5ccccc5)ccc4c4c5c6ccccc6n(-c6cccc(-c7ccccc7)c6)c5ccc43)cc2)cc1 ; c1ccc(-c2ccc(-n3c4ccccc4c4c5c6ccc(-c7ccccc7)cc6n(-c6ccccc6)c5ccc43)cc2)cc1]

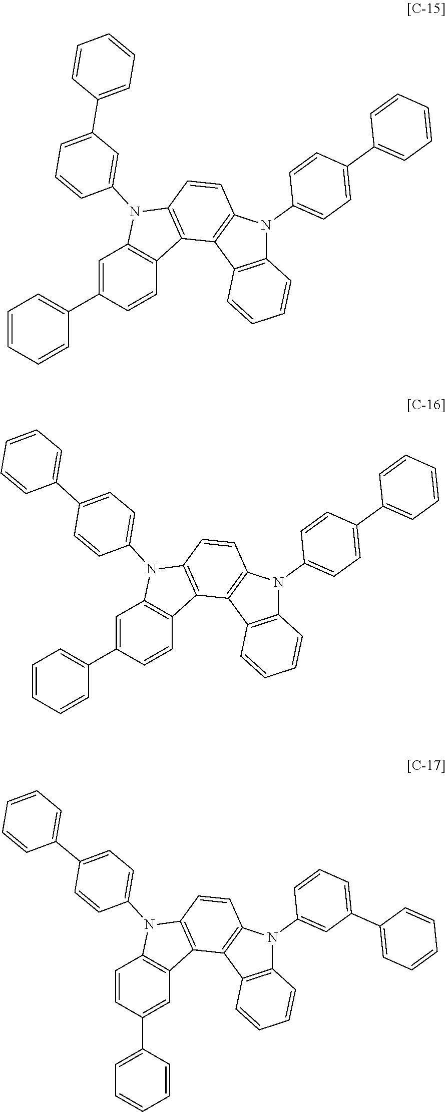 [SMILES: c1ccc(-c2ccc(-n3c4ccccc4c4c5c6ccc(-c7ccccc7)cc6n(-c6cccc(-c7ccccc7)c6)c5ccc43)cc2)cc1 ; c1ccc(-c2ccc(-n3c4ccccc4c4c5c6ccc(-c7ccccc7)cc6n(-c6ccc(-c7ccccc7)cc6)c5ccc43)cc2)cc1 ; c1ccc(-c2ccc(-n3c4ccc(-c5ccccc5)cc4c4c5c6ccccc6n(-c6cccc(-c7ccccc7)c6)c5ccc43)cc2)cc1]

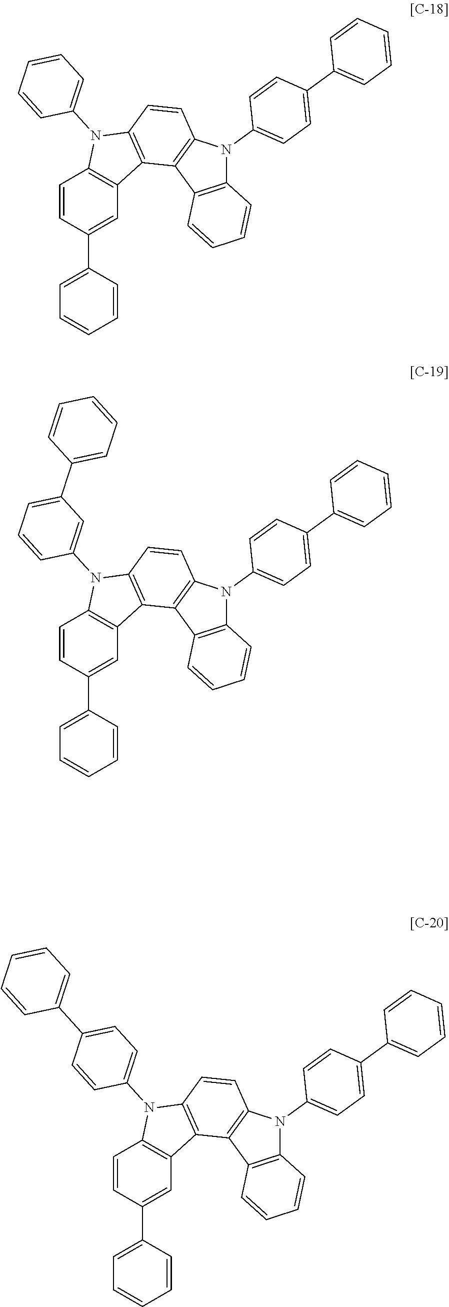 [SMILES: c1ccc(-c2ccc(-n3c4ccccc4c4c5c6cc(-c7ccccc7)ccc6n(-c6ccccc6)c5ccc43)cc2)cc1 ; c1ccc(-c2ccc(-n3c4ccccc4c4c5c6cc(-c7ccccc7)ccc6n(-c6cccc(-c7ccccc7)c6)c5ccc43)cc2)cc1 ; c1ccc(-c2ccc(-n3c4ccccc4c4c5c6cc(-c7ccccc7)ccc6n(-c6ccc(-c7ccccc7)cc6)c5ccc43)cc2)cc1]

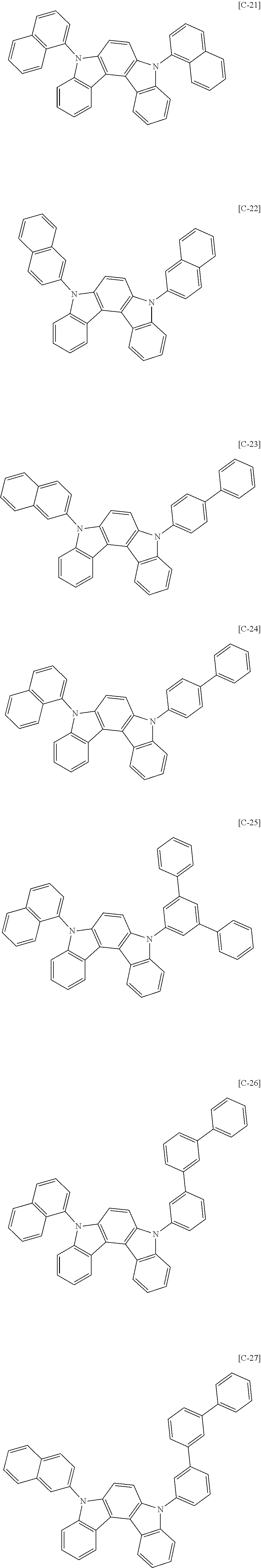

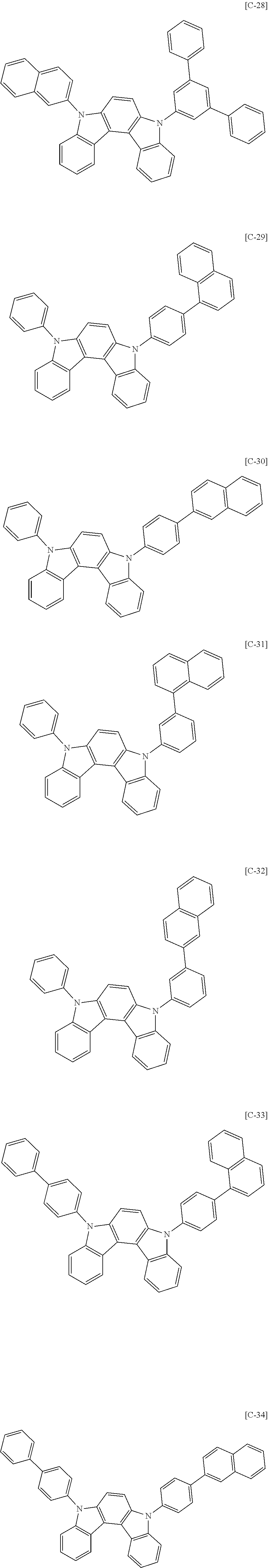

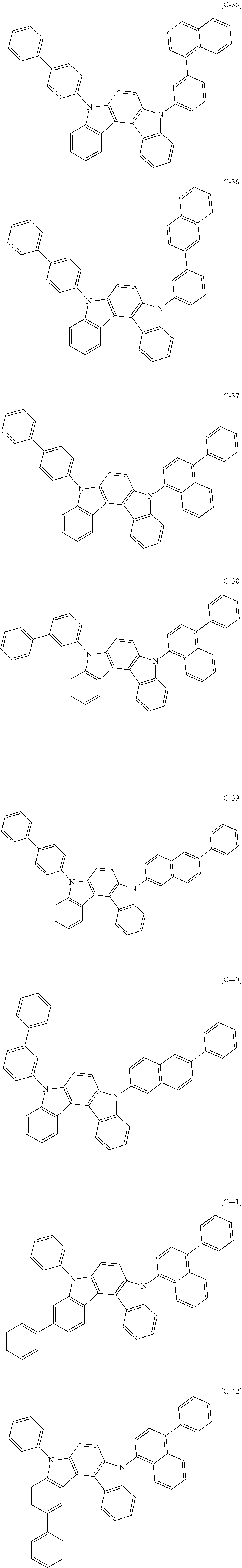

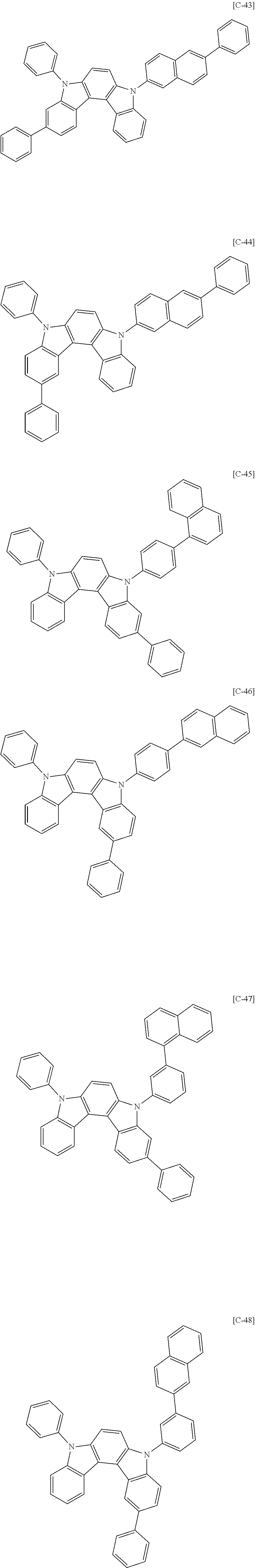 [SMILES: c1ccc(-c2ccc3cc(-n4c5ccccc5c5c6c7ccc(-c8ccccc8)cc7n(-c7ccccc7)c6ccc54)ccc3c2)cc1 ; c1ccc(-c2ccc3cc(-n4c5ccccc5c5c6c7cc(-c8ccccc8)ccc7n(-c7ccccc7)c6ccc54)ccc3c2)cc1 ; c1ccc(-c2ccc3c4c5c6ccccc6n(-c6ccccc6)c5ccc4n(-c4ccc(-c5cccc6ccccc56)cc4)c3c2)cc1 ; c1ccc(-c2ccc3c(c2)c2c4c5ccccc5n(-c5ccccc5)c4ccc2n3-c2ccc(-c3ccc4ccccc4c3)cc2)cc1 ; c1ccc(-c2ccc3c4c5c6ccccc6n(-c6ccccc6)c5ccc4n(-c4cccc(-c5cccc6ccccc56)c4)c3c2)cc1 ; c1ccc(-c2ccc3c(c2)c2c4c5ccccc5n(-c5ccccc5)c4ccc2n3-c2cccc(-c3ccc4ccccc4c3)c2)cc1]

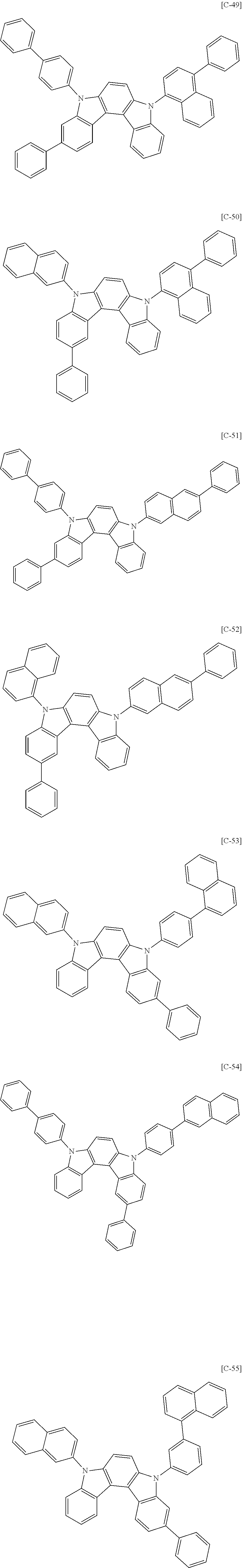

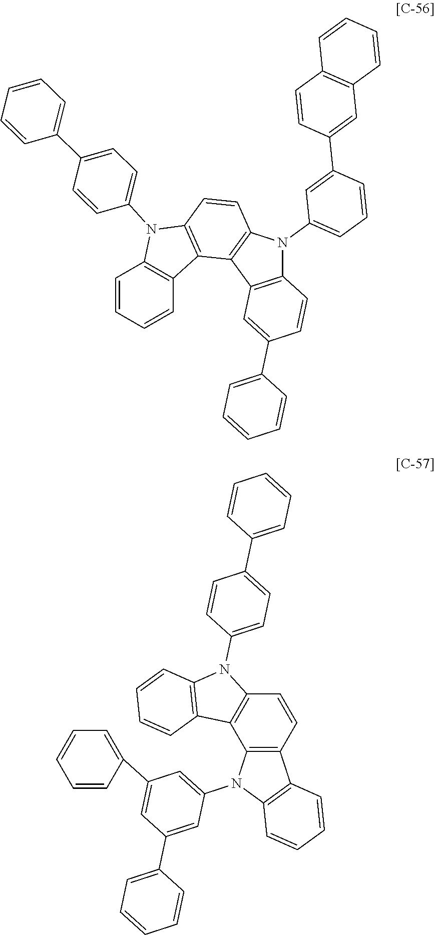

In an implementation, examples of Compound C-1 to Compound C-57 listed in Group 2 in which at least one hydrogen is replaced with deuterium are given below. In an implementation, deuterium may be substituted, e.g., as shown in Compound C-58 to Compound C-72, exemplified below.

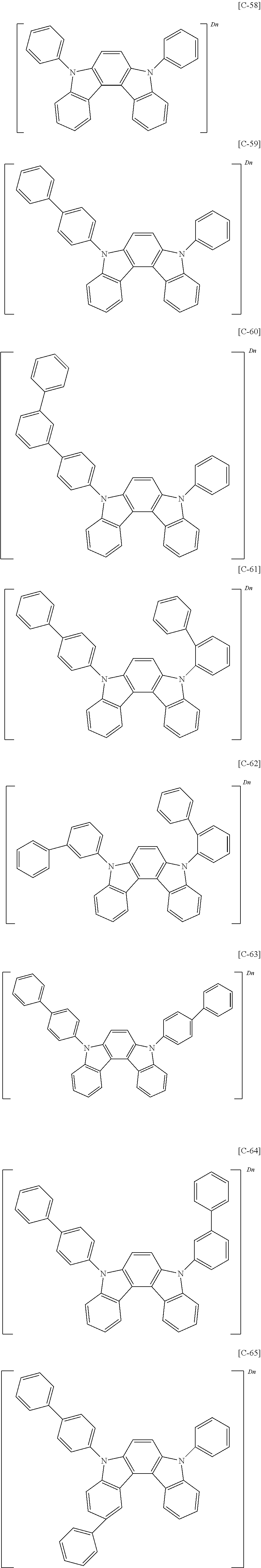

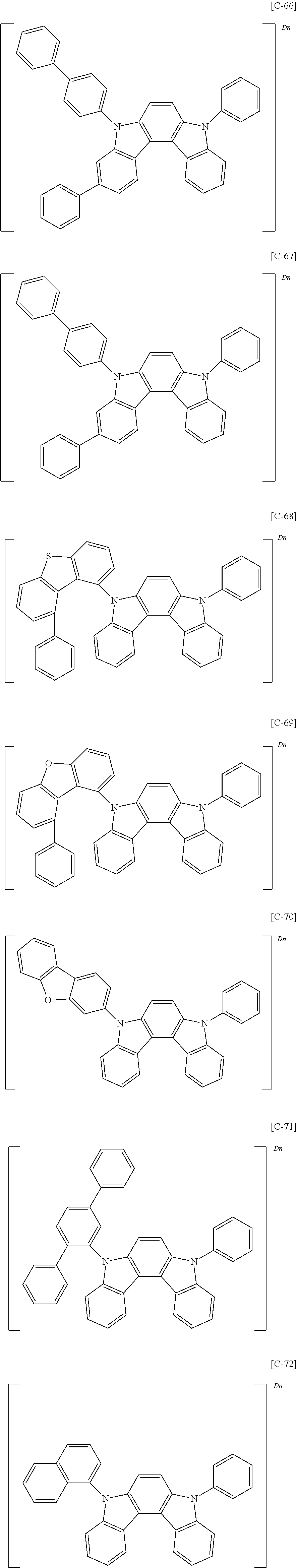

(Dn Refers to the Number of Deuterium Substitutions and Indicates a Structure Substituted with One or More Deuterium Atoms)

The deuterium substitution position and deuterium substitution ratio may include all changeable ranges within the range of Compound C-1 to Compound C-72 (e.g., any hydrogen in any compound may be a protium or a deuterium).

The most specific structures for Compound C-58 to Compound C-72 of Group 2 are presented below as examples according to the position and substitution rate of deuterium substitution.

In an implementation, deuterium may be substituted as shown in Compound C-73 to Compound C-102.

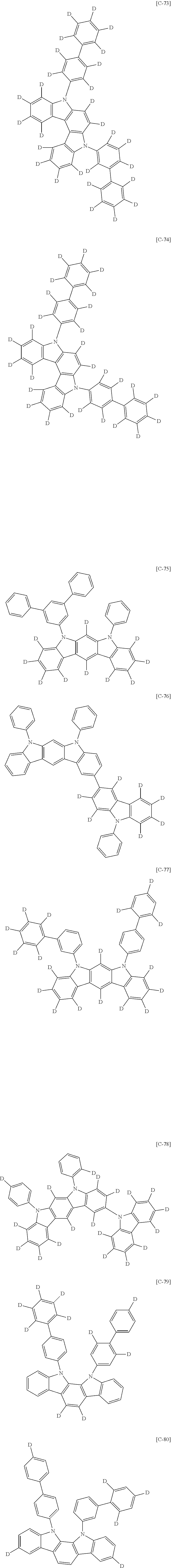

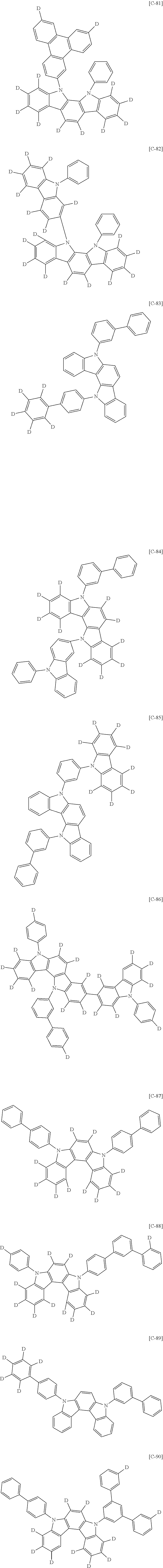

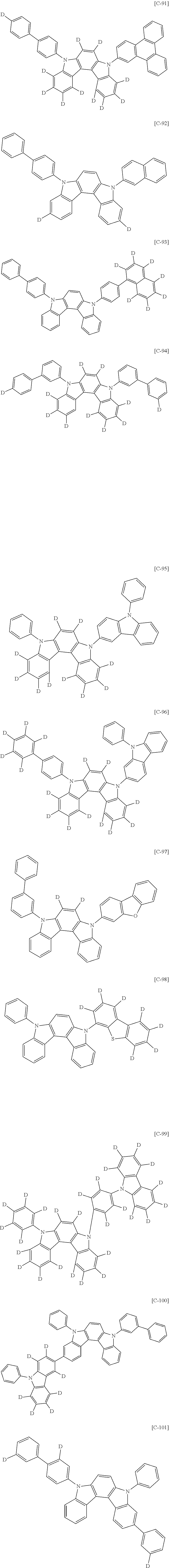

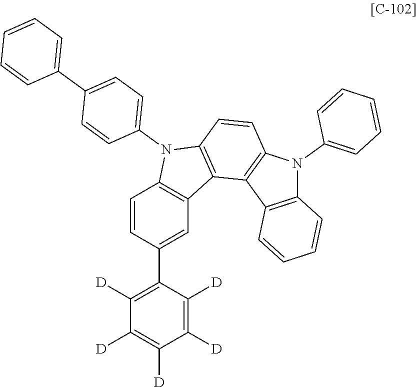 [SMILES: [2H]c1c([2H])c([2H])c(-c2ccc3c(c2)c2c4c5ccccc5n(-c5ccccc5)c4ccc2n3-c2ccc(-c3ccccc3)cc2)c([2H])c1[2H]]

The first compound and the second compound may be included in a weight ratio of, e.g., about 1:99 to about 99:1. By being included in the above range, efficiency and life-span may be improved by implementing bipolar characteristics by adjusting the appropriate weight ratio using the electron transport capability of the first compound and the hole transport capability of the second compound. Within the above range, they may be included in a weight ratio of, e.g., about 10:90 to about 90:10, about 20:80 to about 80:20, e.g., about 20:80 to about 70:30, about 20:80 to about 60:40, and about 30:70 to about 60:40. In an implementation, they may be included in a weight ratio of about 40:60, about 50:50, or about 60:40.

Hereinafter, an organic optoelectronic device including the aforementioned composition for an organic optoelectronic device will be described.

The organic optoelectronic device may be a suitable device to convert electrical energy into photoenergy and vice versa, e.g., an organic photoelectric device, an organic light emitting diode, an organic solar cell, or an organic photoconductor drum.

Herein, an organic light emitting diode as one example of an organic optoelectronic device is described referring to the drawing.

The FIG. is a cross-sectional view showing an organic light emitting diode according to some example embodiments.

Referring to the FIGURE, an organic light emitting diode 100 according to some example embodiments may include an anode 120 and a cathode 110 facing each other and an organic layer 105 between the anode 120 and cathode 110.

The anode 120 may be made of a conductor having a large work function to help hole injection, and may be, e.g., a metal, a metal oxide, or a conductive polymer. The anode 120 may be, e.g., a metal such as nickel, platinum, vanadium, chromium, copper, zinc, gold, or the like or an alloy thereof; a metal oxide such as zinc oxide, indium oxide, indium tin oxide (ITO), indium zinc oxide (IZO), or the like; a combination of a metal and an oxide such as ZnO and Al or SnO₂ and Sb; a conductive polymer such as poly(3-methylthiophene), poly(3,4-(ethylene-1,2-dioxy)thiophene) (PEDOT), polypyrrole, or polyaniline.

The cathode 110 may be made of a conductor having a small work function to help electron injection, and may be, e.g., a metal, a metal oxide, or a conductive polymer. The cathode 110 may be, e.g., a metal such as magnesium, calcium, sodium, potassium, titanium, indium, yttrium, lithium, gadolinium, aluminum, silver, tin, lead, cesium, barium, or the like, or an alloy thereof; a multi-layer structure material such as LiF/Al, LiO₂/Al, LiF/Ca, and BaF₂/Ca.

The organic layer 105 may include the aforementioned composition for an organic optoelectronic device.

The organic layer 105 may include a light emitting layer 130, and the light emitting layer 130 may include a host and a dopant, the host may include the aforementioned composition for an organic optoelectronic device, and the dopant may be, e.g., a phosphorescent dopant, e.g., a red, green or blue phosphorescent dopant, e.g., a red or green phosphorescent dopant.

The dopant may be a material mixed with the composition for an organic optoelectronic device in a small amount to cause light emission and may be generally a material such as a metal complex that emits light by multiple excitation into a triplet or more. The dopant may be, e.g., an inorganic, organic, or organic-inorganic compound, and one or more types thereof may be used.

Examples of the dopant may be or include a phosphorescent dopant and examples of the phosphorescent dopant may be or include an organic metal compound including Ir, Pt, Os, Ti, Zr, Hf, Eu, Tb, Tm, Fe, Co, Ni, Ru, Rh, Pd, or a combination thereof. The phosphorescent dopant may be, e.g., a compound represented by Chemical Formula Z.

L⁶MX¹ [Chemical Formula Z]

In Chemical Formula Z, M may be, e.g., a metal and L⁶ and X¹ may be the same or different and may be, e.g., a ligand forming a complex compound with M.

The M may be, e.g., Ir, Pt, Os, Ti, Zr, Hf, Eu, Tb, Tm, Fe, Co, Ni, Ru, Rh, Pd, or a combination thereof, and L⁶ and X¹ may be, e.g., a bidentate ligand.

The ligands represented by L⁶ and X¹ may be, e.g., one of Chemical Formula Z-1 to Chemical Formula Z-8.

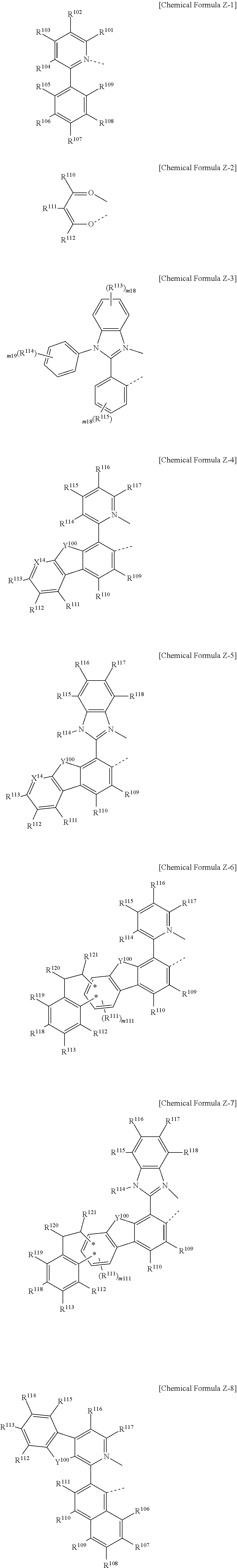

In Chemical Formula Z-1 to Chemical Formula Z-8, X¹⁴ may be, e.g., carbon or nitrogen.

Y¹⁰⁰ may be, e.g., O or S.

R¹⁰¹ to R¹²² may each independently be, e.g., hydrogen, deuterium, a halogen, a substituted or unsubstituted C1 to C30 alkyl group, a substituted or unsubstituted C3 to C30 cycloalkyl group, a substituted or unsubstituted C6 to C20 aryl group, —SiR¹³³R¹³⁴R¹³⁵, or —GeR¹³³R¹³⁴R¹³⁵; or may be linked to adjacent substituents to form a substituted or unsubstituted ring, and, e.g., together with pyridine may form a substituted or unsubstituted quinoline, a substituted or unsubstituted benzofuropyridine, a substituted or unsubstituted benzothienopyridine, a substituted or unsubstituted indenopyridine, a substituted or unsubstituted benzofuroquinoline, a substituted or unsubstituted benzothienoquinoline, or a substituted or unsubstituted indenoquinoline.

m18 may be, e.g., an integer of 1 to 4.

m19 may be, e.g., an integer of 1 to 5.

m111 may be, e.g., an integer of 1 to 2.

R¹³³ to R¹³⁵ are each independently a C1 to C6 alkyl group.

Examples of ligands represented by L⁶ and X¹ may be, e.g., a chemical formula of Group A.

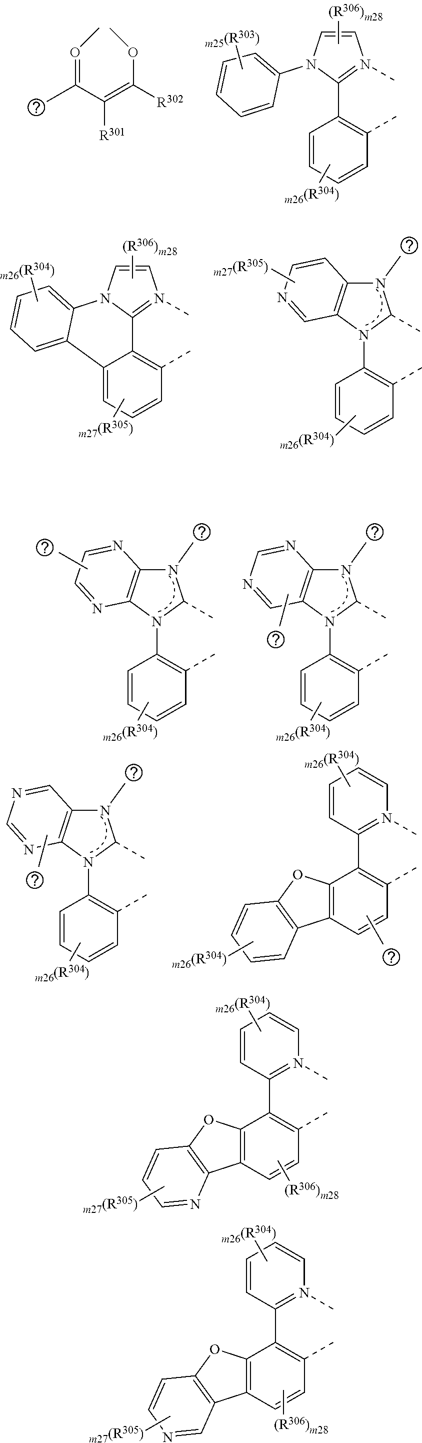

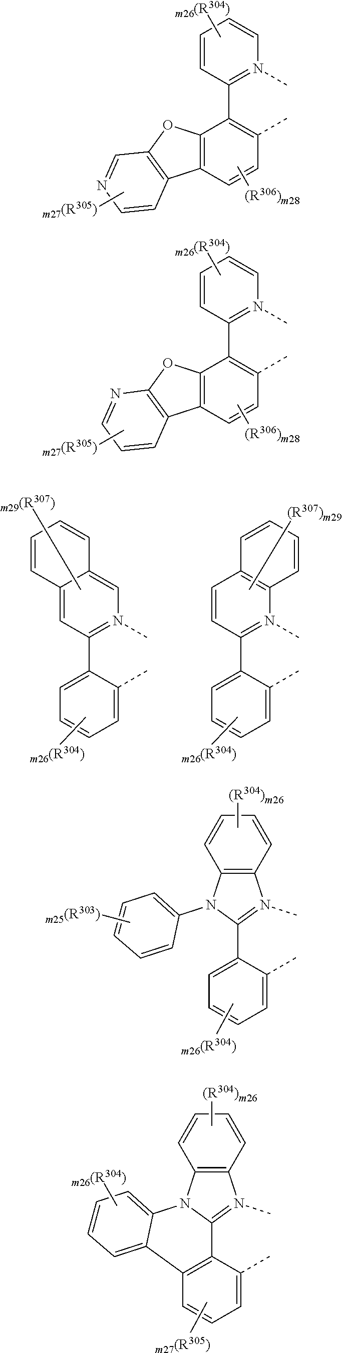

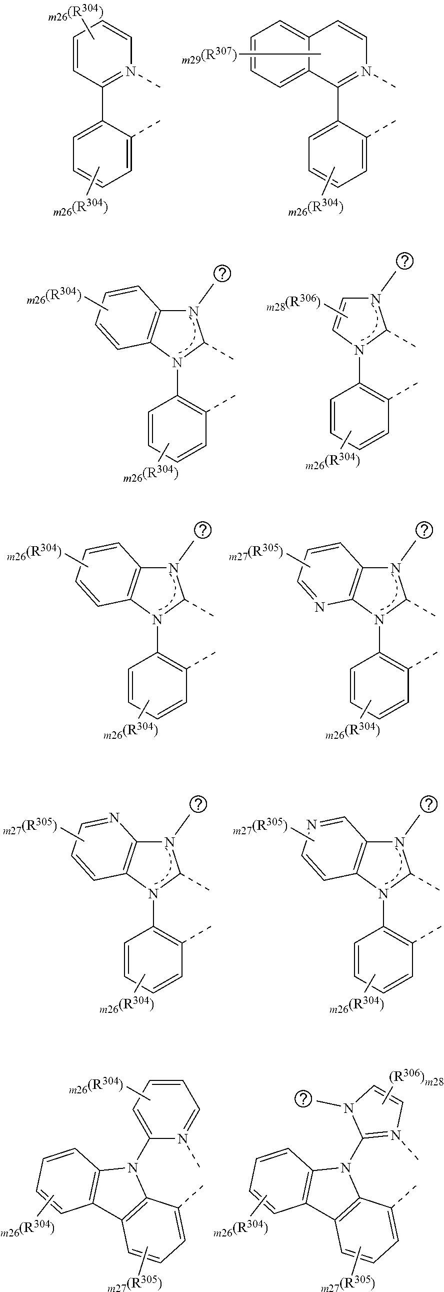

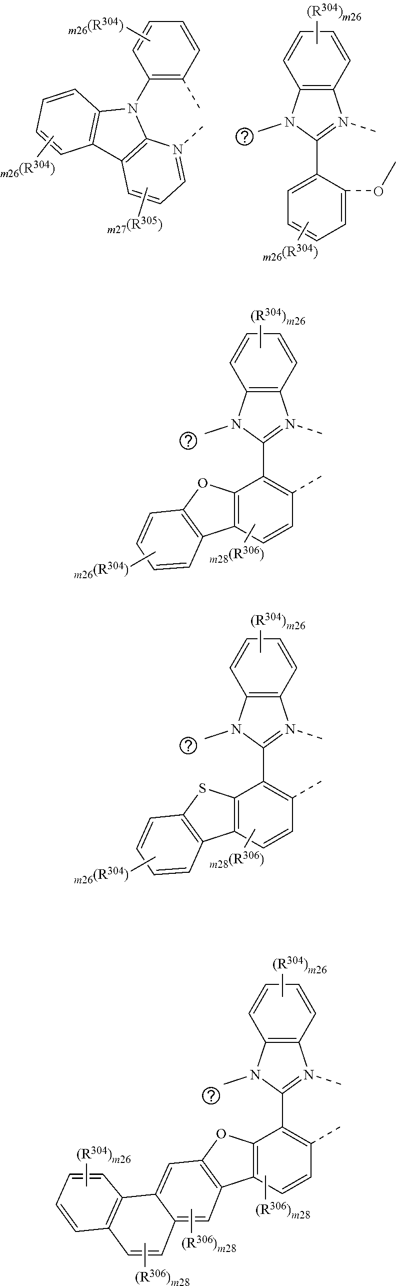

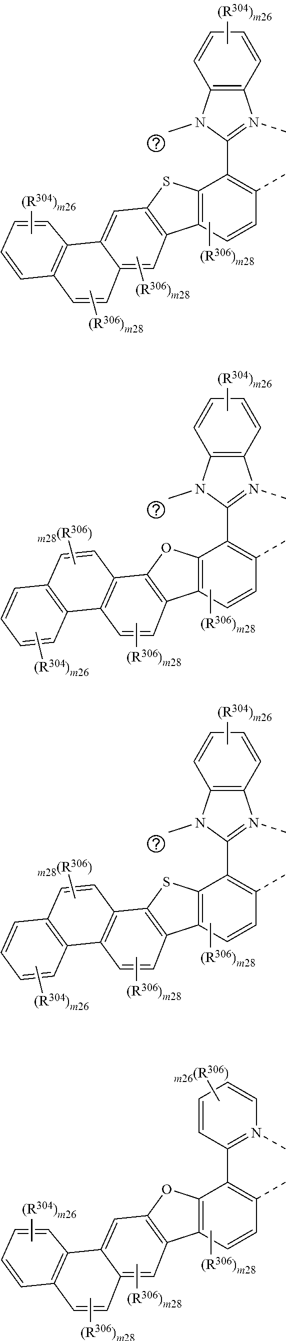

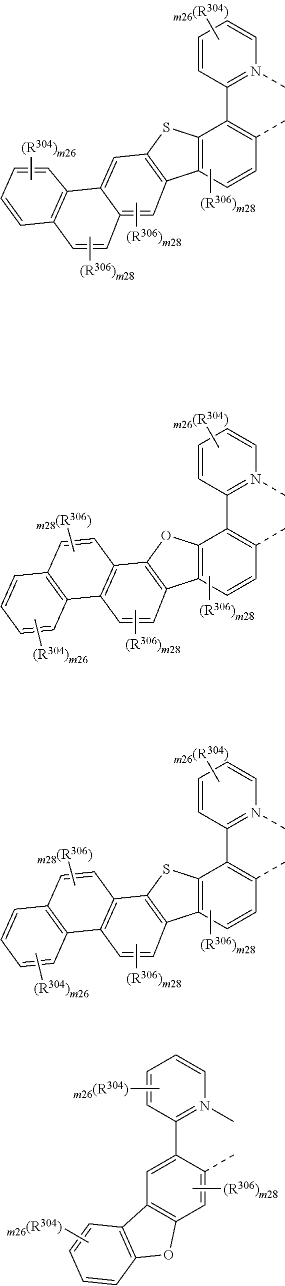

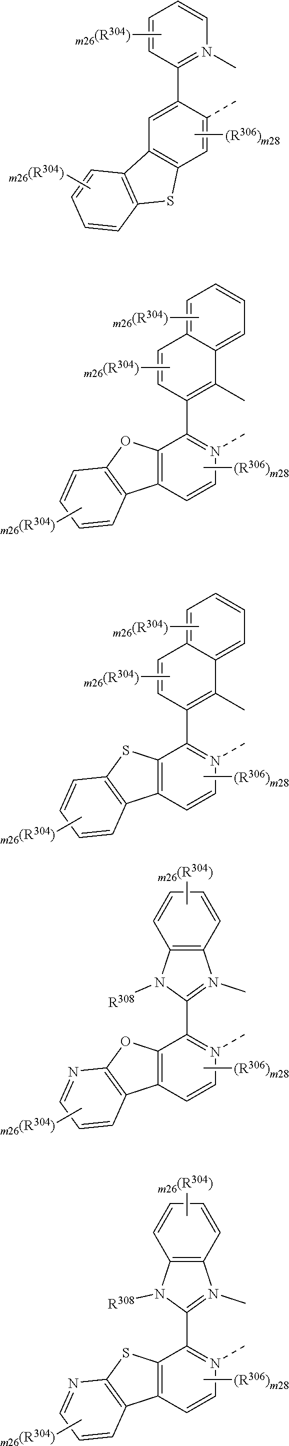

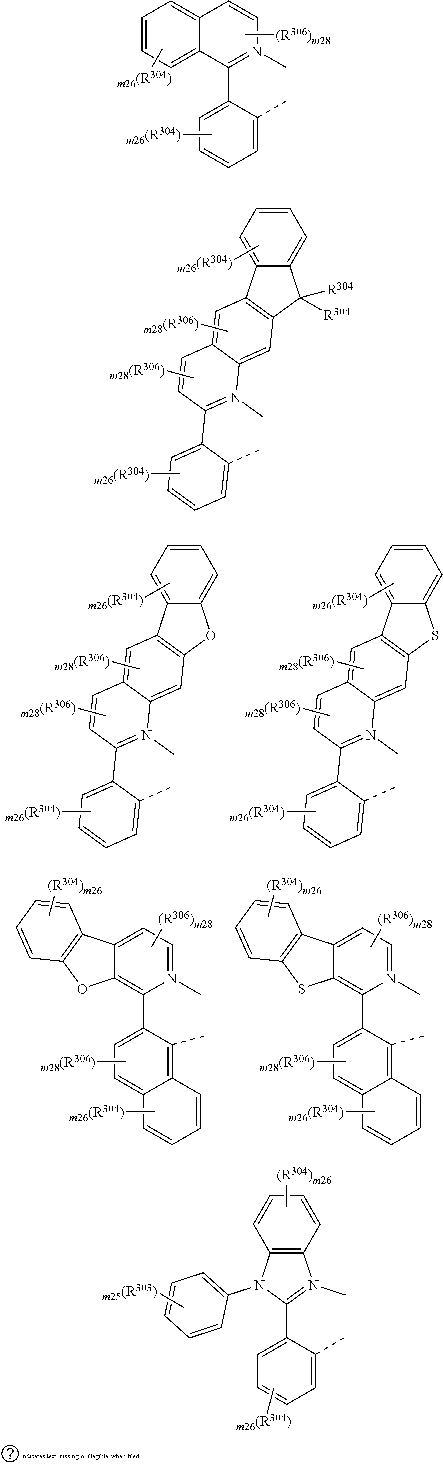

In Group A, R³⁰⁰ to R³⁰² may each independently be, e.g., hydrogen, deuterium, a C to C30 alkyl group substituted or unsubstituted with a halogen, a C6 to C30 aryl group substituted or unsubstituted with C1 to C30 alkyl, or a halogen.

R³⁰³ to R³⁰¹ may each independently be, e.g., hydrogen, deuterium, a halogen, a substituted or unsubstituted C1 to C30 alkyl group, a substituted or unsubstituted C1 to C30 alkoxy group, a substituted or unsubstituted C₃ to C30 cycloalkyl group, a substituted or unsubstituted C2 to C30 alkenyl group, a substituted or unsubstituted C6 to C30 aryl group, a substituted or unsubstituted C1 to C30 heteroaryl group, a substituted or unsubstituted C1 to C30 amino group, a substituted or unsubstituted C6 to C30 arylamino group, SF₅, a trialkylsilyl group having a substituted or unsubstituted C1 to C30 alkyl group, a dialkylarylsilyl group having a substituted or unsubstituted C1 to C30 alkyl group and a C6 to C30 aryl group, or a triarylsilyl group having a substituted or unsubstituted C6 to C30 aryl group.

m25 may be, e.g., an integer of 1 to 5.

m26 may be, e.g., an integer of 1 to 4.

m27 may be, e.g., an integer of 1 to 3.

m28 may be, e.g., an integer of 1 or 2.

m29 may be, e.g., an integer of 1 to 6.

In an implementation, any one of m25 to m27 and m29 may be an integer of 2 or more and m28 may be 2, and the respective ones of R³⁰³ to R³⁰⁷ may be the same or different from each other.

The dopant according to some example embodiments may be an iridium complex, and may be represented, e.g., by one of Chemical Formula 7 to Chemical Formula 9.

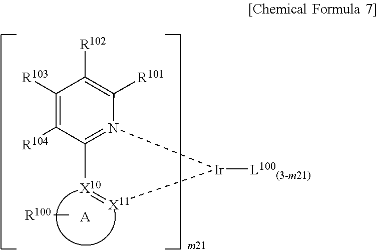

In Chemical Formula 7, ring A may be, e.g., a monocyclic ring or a polycyclic fused ring, wherein each ring of the monocyclic ring and polycyclic fused ring may be, e.g., a 5- or 6-membered carbocyclic or heterocyclic ring.

R¹⁰⁰ may represent, e.g., one to a maximum number of monovalent substituents.

In an implementation, there may be two or more R¹⁰⁰ groups, and each R¹⁰⁰ may be the same or different from each other.

R¹⁰⁰ to R¹⁰⁴ may each independently be, e.g., hydrogen, deuterium, a halogen, a substituted or unsubstituted C1 to C10 alkyl group, a substituted or unsubstituted C6 to C20 aryl group, —SiR¹¹⁴R¹¹⁵R¹¹⁶, —GeR¹¹⁴R¹¹⁵R¹¹⁶, or a combination thereof.

R¹¹⁴ to R¹¹⁶ may each independently be, e.g., a substituted or unsubstituted C1 to C6 alkyl group.

X¹⁰ and X¹¹ may each independently be, e.g., carbon or nitrogen.

L¹⁰⁰ may be, e.g., a ligand of a monovalent anion or a bidentate ligand, which coordinates to iridium through the unshared electron pair of carbon or a heteroatom.

m21 may be, e.g., an integer of 0 to 3.

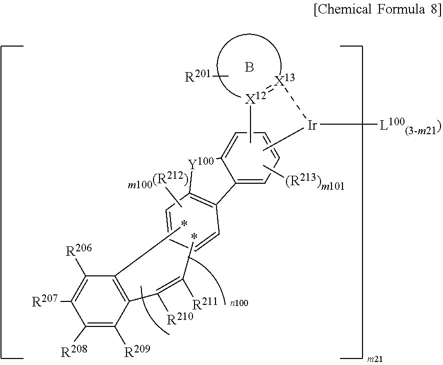

In Chemical Formula 8, ring B may be, e.g., a monocyclic ring or a polycyclic fused ring, wherein each ring of the monocyclic ring and polycyclic fused ring may be, e.g., a 5- or 6-membered carbocyclic or heterocyclic ring.

Y¹⁰⁰ may be, e.g., O or S.

R²⁰¹ may represent, e.g., one to a maximum number of monovalent substituents.

In an implementation, there may be two or more R²⁰¹ groups, and each R²⁰¹ may be the same or different from each other.

R²⁰¹ and R²⁰⁶ to R²¹³ may each independently be, e.g., hydrogen, deuterium, a halogen, a substituted or unsubstituted C1 to C10 alkyl group, a substituted or unsubstituted C6 to C20 aryl group, —SiR¹¹⁴R¹¹⁵R¹¹⁶, —GeR¹¹⁴R¹¹⁵R¹¹⁶, or a combination thereof.

R¹¹⁴ to R¹¹⁶ may each independently be, e.g., a substituted or unsubstituted C1 to C6 alkyl group.

X¹² and X¹³ may each independently be, e.g., carbon or nitrogen.

L¹⁰⁰ may be, e.g., a ligand of a monovalent anion or a bidentate ligand, which coordinates to iridium through the unshared electron pair of carbon or a heteroatom.

m21 may be, e.g., an integer of 0 to 3.

m100 may be, e.g., an integer of 1 to 3.

m101 may be, e.g., an integer of 1 or 2.

n100 may be, e.g., an integer of 0 or 1.

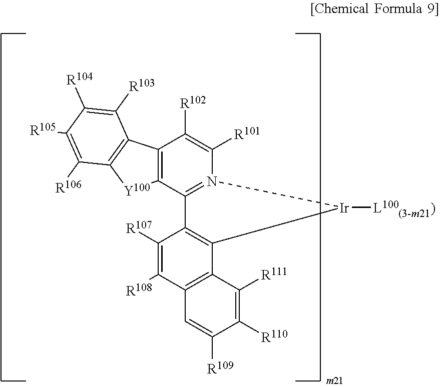

In Chemical Formula 9, Y¹⁰⁰ may be, e.g., O or S.

R¹⁰¹ to R¹¹¹ may each independently be, e.g., hydrogen, deuterium, a halogen, a substituted or unsubstituted C1 to C10 alkyl group, a substituted or unsubstituted C6 to C20 aryl group, —SiR¹¹⁴R¹¹⁵R¹¹⁶, —GeR¹¹⁴R¹¹⁵R¹¹⁶, or a combination thereof.

R¹¹⁴ to R¹¹⁶ may each independently be, e.g., a substituted or unsubstituted C1 to C6 alkyl group.

L¹⁰⁰ may be, e.g., a ligand of a monovalent anion or a bidentate ligand, which coordinates to iridium through the unshared electron pair of carbon or a heteroatom.

m21 may be, e.g., an integer of 0 to 3.

In an implementation, the iridium complex may be represented by, e.g., one of Chemical Formula 7-1 to Chemical Formula 7-6.

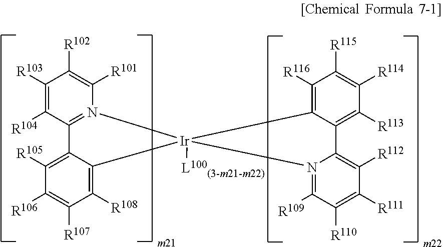

In Chemical Formula 7-1, R¹⁰¹ to R¹¹⁶ may each independently be, e.g., hydrogen, deuterium, a halogen, a substituted or unsubstituted C1 to C10 alkyl group, a substituted or unsubstituted C6 to C20 aryl group, —SiR¹³²R¹³³R¹³⁴, or —GeR¹³²R¹³³R¹³⁴.

R¹³² to R¹³⁴ may each independently be, e.g., a substituted or unsubstituted C1 to C6 alkyl group.

At least one of R¹⁰¹ to R¹¹⁶ may be, e.g., a functional group represented by Chemical Formula V-1.

L¹⁰° may be, e.g., a bidentate ligand of a monovalent anion and may be, e.g., a ligand that coordinates to iridium through a lone pair of carbons or heteroatoms.

m21 and m22 may each independently be, e.g., an integer of 0 to 3 and m21+m22 may be, e.g., an integer of 1 to 3.

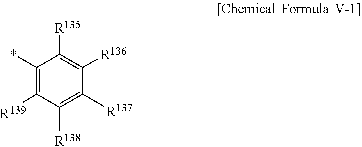

In Chemical Formula V-1, R¹³⁵ to R¹³⁹ may each independently be, e.g., hydrogen, deuterium, a substituted or unsubstituted C1 to C10 alkyl group, a substituted or unsubstituted C6 to C20 aryl group, or —SiR¹³²R¹³³R¹³⁴.

R¹³² to R¹³⁴ may each independently be, e.g., a substituted or unsubstituted C1 to C6 alkyl group.

* refers to a portion linked to a carbon atom.

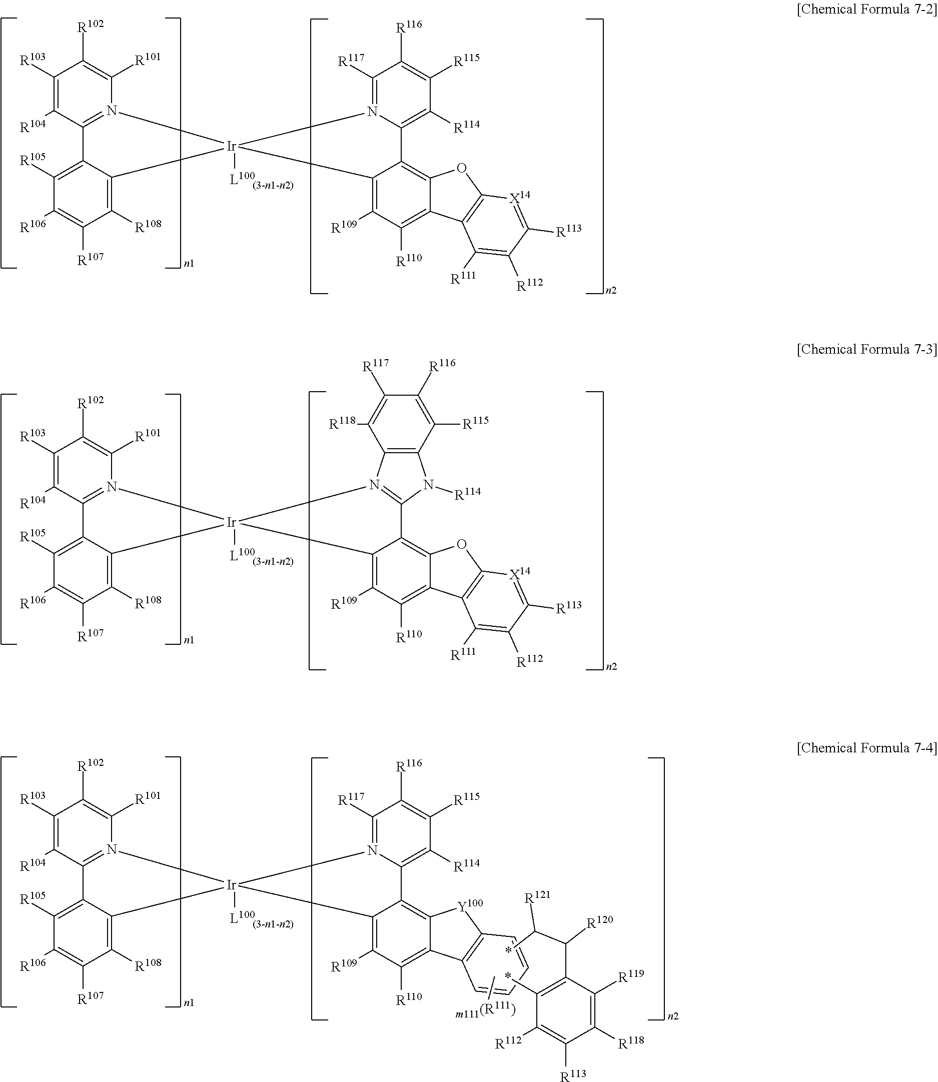

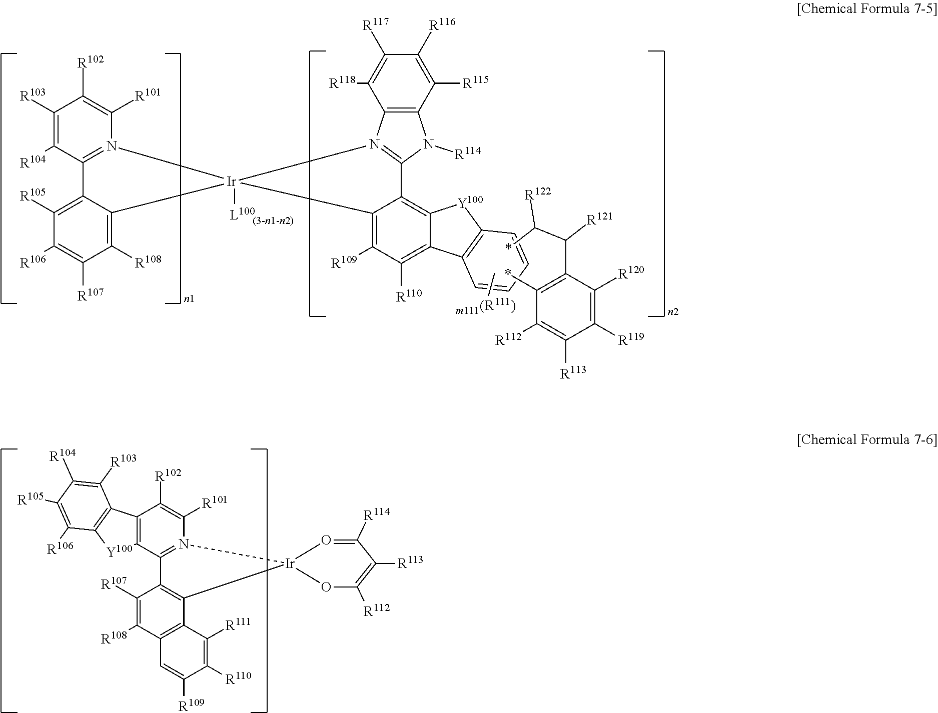

In Chemical Formula 7-2 to Chemical Formula 7-6, X¹⁴ may be, e.g., carbon or nitrogen.

Y¹⁰⁰ may be. e.g. O or S.

R¹⁰¹ to R¹²² may each independently be, e.g., hydrogen, deuterium, a halogen, a substituted or unsubstituted C1 to C10 alkyl group, a substituted or unsubstituted C6 to C20 aryl group, —SiR¹³³R¹³⁴R¹³⁵ or —GeR¹³³R¹³⁴R¹³⁵.

R¹³³ to R¹³⁵ may each independently be, e.g., a substituted or unsubstituted C1 to C6 alkyl group.

L¹⁰⁰ may be, e.g., bidentate ligand of a monovalent anion and may be, e.g., a ligand that coordinates to iridium through a lone pair of carbons or heteroatoms.

m111 may be, e.g., an integer of 1 to 2.

n1 and n2 may each independently be, e.g., an integer of 0 to 3 and n1+n2 may be, e.g., an integer of 1 to 3.

The dopant according to an implementation may be, e.g., a platinum complex, and may be represented, e.g., by Chemical Formula Z-9.

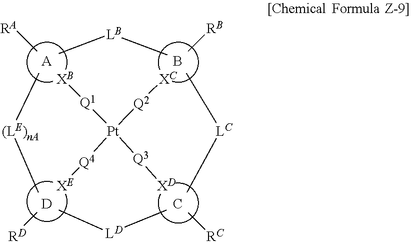

In Chemical Formula Z-9, rings A, B, C, and D may each independently be, e.g., a 5-membered or 6-membered carbocyclic or heterocyclic ring.

R^{A}, R^{B}, R^{C}, and R^{D} may each independently be, e.g., mono-, di-, tri-, or tetra-substitution, or unsubstitution.

nA may be, e.g., an integer of 0 or 1.

L^{B}, L^{C}, and L^{D} may each independently be, e.g., a direct bond, BR, NR, PR, O, S, Se, C═O, S═O, SO₂, CRR′, SiRR′, GeRR′, or a combination thereof.

In an implementation, nA may be 1, and L^{E} may be, e.g., a direct bond, BR, NR, PR, O, S, Se, C═O, S═O, SO₂, CRR′, SiRR′, GeRR′, or a combination thereof.

In an implementation, nA may be 0 and L^{E} may not exist.

R^{A}, R^{B}, R^{C}, R^{D}, R, and R′ may each independently be, e.g., hydrogen, deuterium, a halogen, an alkyl group, a cycloalkyl group, a heteroalkyl group, an arylalkyl group, an alkoxy group, an aryloxy group, an amino group, a silyl group, an alkenyl group, a cycloalkenyl group, a heteroalkenyl group, an alkynyl group, an aryl group, a heteroaryl group, an acyl group, a carbonyl group, a carboxylic acid group, an ester group, a nitrile group, an isonitrile group, a sulfanyl group, a sulfinyl group, a sulfonyl group, a phosphino group, or a combination thereof, any adjacent R^{A}, R^{B}, R^{C}, R^{D}, R, and R′ may be optionally linked to each other to provide a ring; X^{B}, X^{c}, X^{D}, and X^{E} may each independently be, e.g., carbon or nitrogen; and Q¹, Q², Q³, and Q⁴ may each independently be, e.g., oxygen or a direct bond.

The platinum complex may be represented by, e.g., Chemical Formula 8-1 or Chemical Formula 8-2.

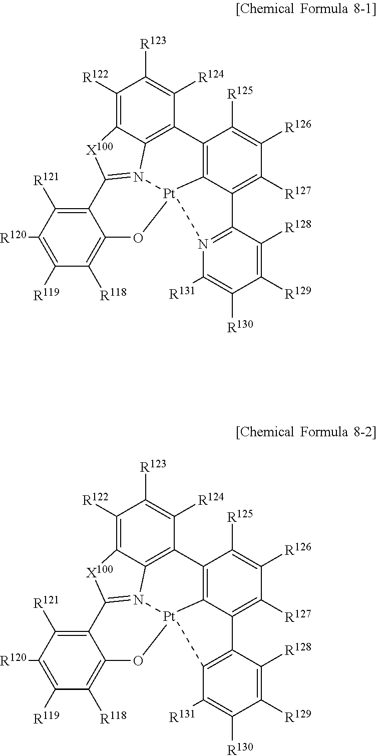

In Chemical Formula 8-1 and Chemical Formula 8-2. X¹⁰⁰ may be, e.g., O. S. or NR¹³².

R¹¹⁸ to R¹³² may each independently be, e.g., hydrogen, deuterium, a substituted or unsubstituted C1 to C10 alkyl group, a substituted or unsubstituted C6 to C20 aryl group, or —SiR¹³³R¹³⁴R¹³⁵.

R¹³³ to R¹³⁵ may each independently be, e.g., a substituted or unsubstituted C1 to C6 alkyl group.

In an implementation, at least one of R¹¹⁸ to R¹³² may be, e.g., —SiR¹³³R¹³⁴R¹³⁵ or a tert-butyl group.

R¹³³ to R¹³⁵ may each independently be, e.g., a substituted or unsubstituted C1 to C6 alkyl group.

The organic layer may further include a charge transport region in addition to the light emitting layer.

The charge transport region may be, e.g., the hole transport region 140.

The hole transport region 140 may help further increase hole injection or hole mobility between the anode 120 and the light emitting layer 130 and block electrons.

In an implementation, the hole transport region 140 may include, e.g., a hole transport layer between the anode 120 and the light emitting layer 130, and a hole transport auxiliary layer between the light emitting layer 130 and the hole transport layer, and a compound of Group B may be included in at least one of the hole transport layer and the hole transport auxiliary layer.

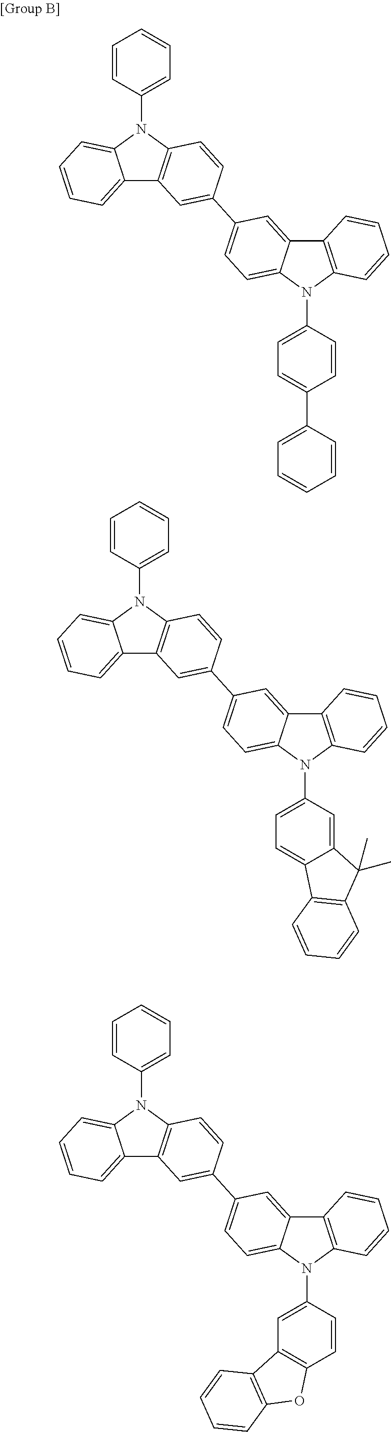 [SMILES: c1ccc(-c2ccc(-n3c4ccccc4c4cc(-c5ccc6c(c5)c5ccccc5n6-c5ccccc5)ccc43)cc2)cc1 ; CC1(C)c2ccccc2-c2ccc(-n3c4ccccc4c4cc(-c5ccc6c(c5)c5ccccc5n6-c5ccccc5)ccc43)cc21 ; c1ccc(-n2c3ccccc3c3cc(-c4ccc5c(c4)c4ccccc4n5-c4ccc5oc6ccccc6c5c4)ccc32)cc1]

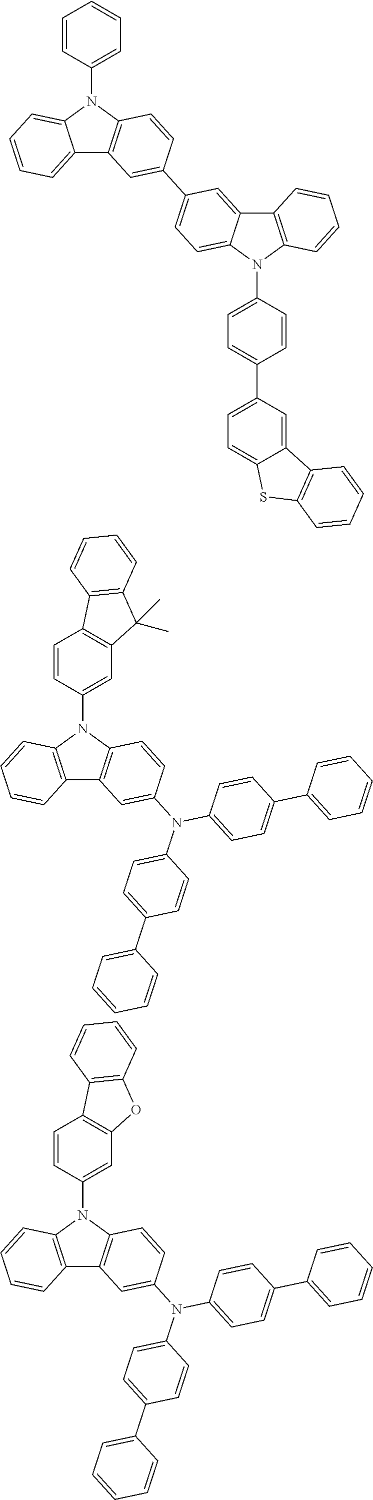

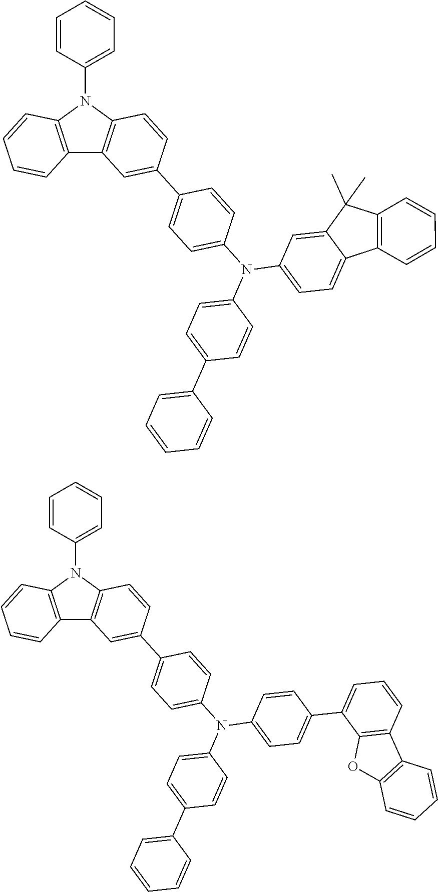 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(-c2ccc(N(c3ccc(-c4ccc5c(c4)c4ccccc4n5-c4ccccc4)cc3)c3ccc(-c4cccc5c4oc4ccccc45)cc3)cc2)cc1]

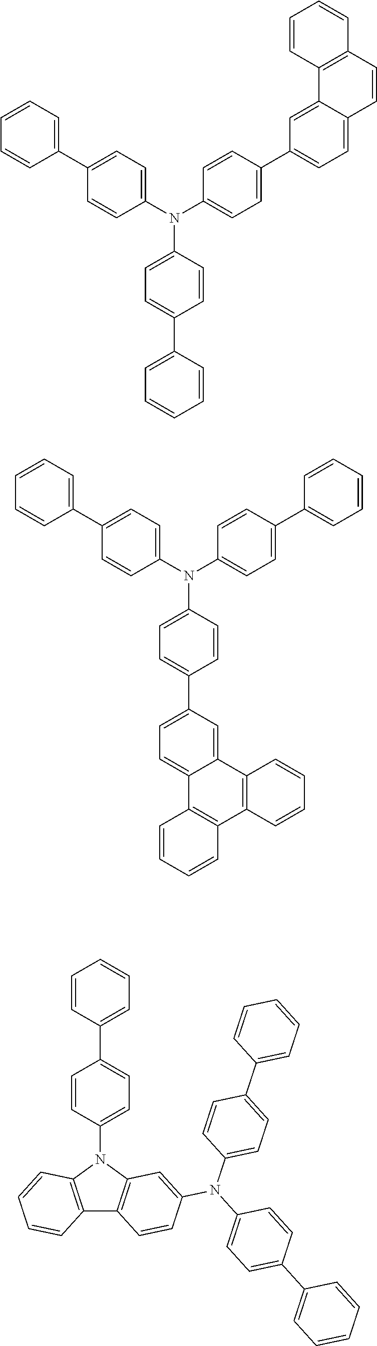 [SMILES: c1ccc(-c2ccc(N(c3ccc(-c4ccccc4)cc3)c3ccc(-c4ccc5ccc6ccccc6c5c4)cc3)cc2)cc1 ; c1ccc(-c2ccc(N(c3ccc(-c4ccccc4)cc3)c3ccc(-c4ccc5c6ccccc6c6ccccc6c5c4)cc3)cc2)cc1 ; c1ccc(-c2ccc(N(c3ccc(-c4ccccc4)cc3)c3ccc4c5ccccc5n(-c5ccc(-c6ccccc6)cc5)c4c3)cc2)cc1]

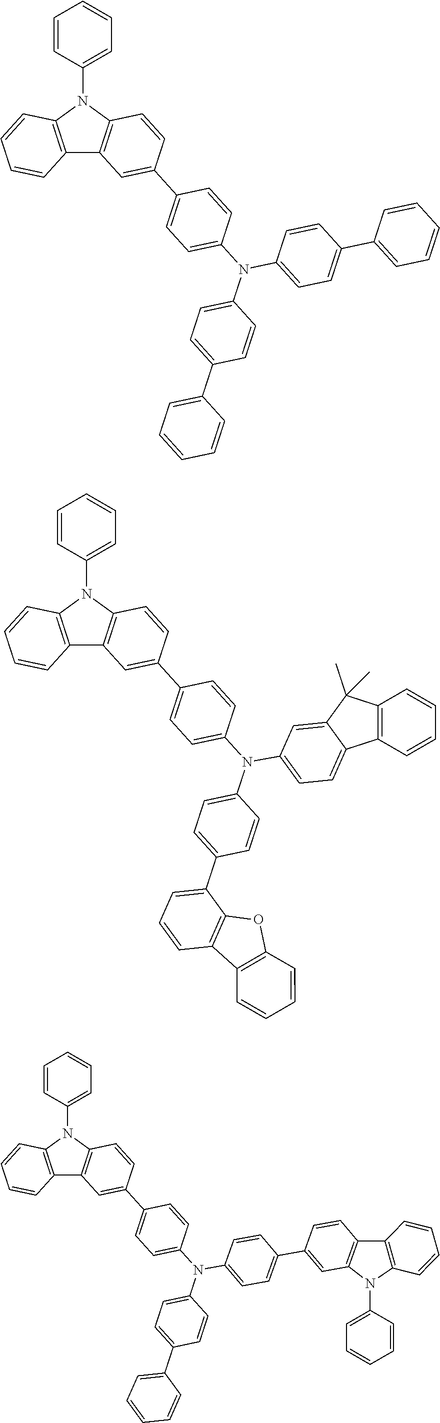 [SMILES: c1ccc(-c2ccc(N(c3ccc(-c4ccccc4)cc3)c3ccc(-c4ccc5c(c4)c4ccccc4n5-c4ccccc4)cc3)cc2)cc1 ; CC1(C)c2ccccc2-c2ccc(N(c3ccc(-c4ccc5c(c4)c4ccccc4n5-c4ccccc4)cc3)c3ccc(-c4cccc5c4oc4ccccc45)cc3)cc21 ; c1ccc(-c2ccc(N(c3ccc(-c4ccc5c(c4)c4ccccc4n5-c4ccccc4)cc3)c3ccc(-c4ccc5c6ccccc6n(-c6ccccc6)c5c4)cc3)cc2)cc1]

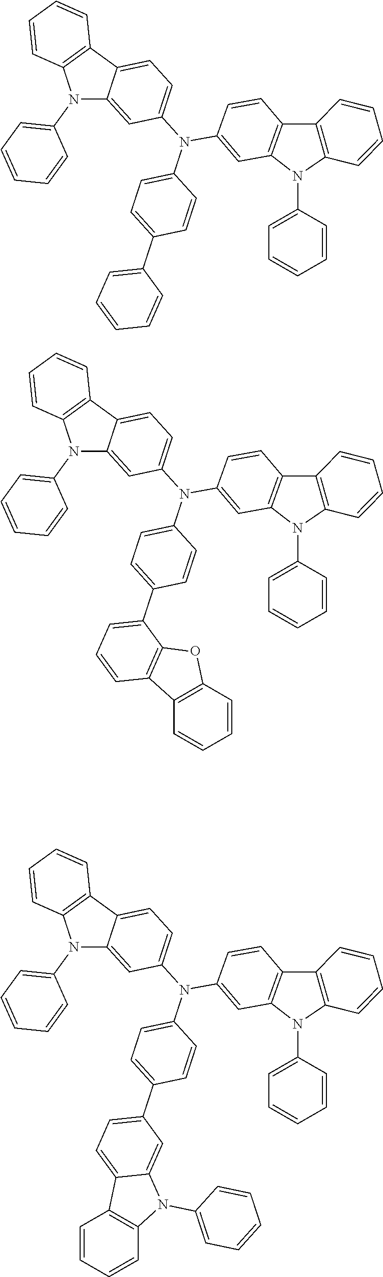 [SMILES: c1ccc(-c2ccc(N(c3ccc4c5ccccc5n(-c5ccccc5)c4c3)c3ccc4c5ccccc5n(-c5ccccc5)c4c3)cc2)cc1 ; c1ccc(-n2c3ccccc3c3ccc(N(c4ccc(-c5cccc6c5oc5ccccc56)cc4)c4ccc5c6ccccc6n(-c6ccccc6)c5c4)cc32)cc1 ; c1ccc(-n2c3ccccc3c3ccc(-c4ccc(N(c5ccc6c7ccccc7n(-c7ccccc7)c6c5)c5ccc6c7ccccc7n(-c7ccccc7)c6c5)cc4)cc32)cc1]

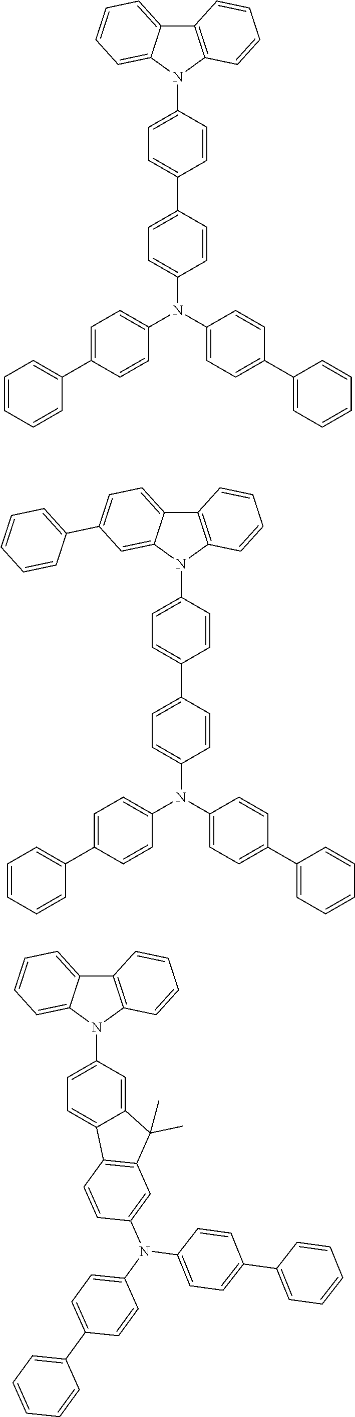

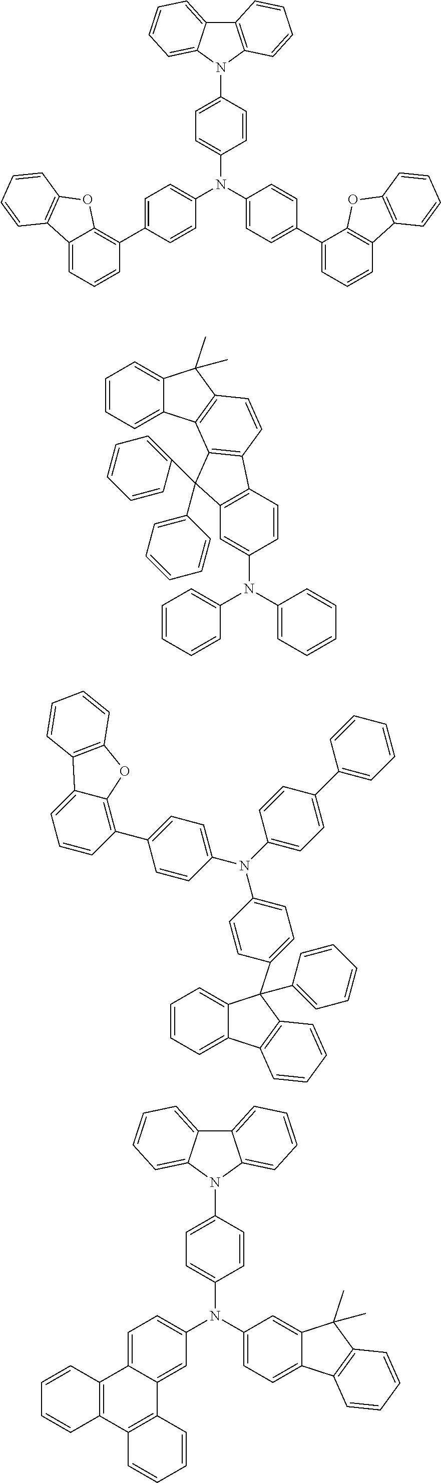

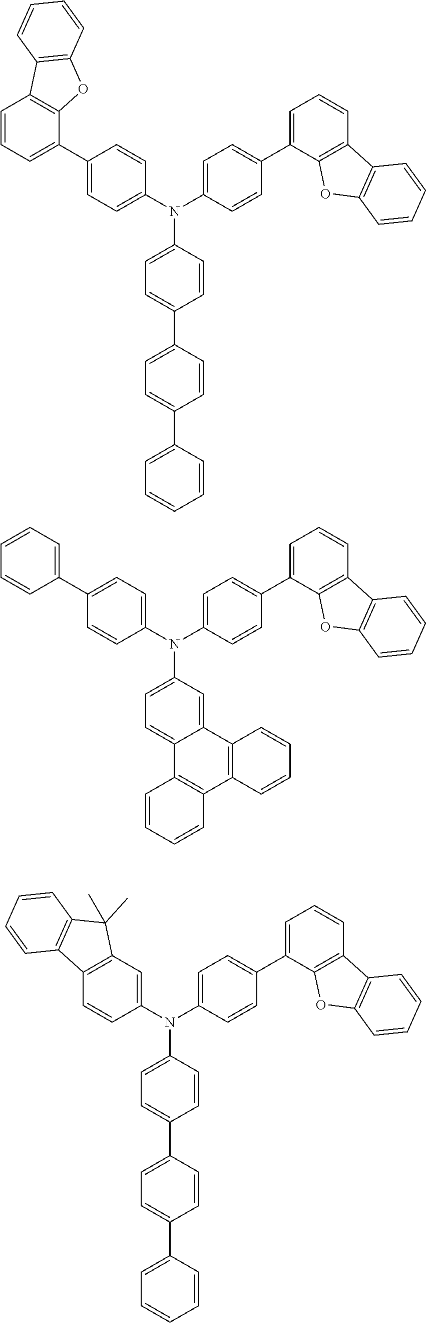 [SMILES: c1ccc(-c2ccc(-c3ccc(N(c4ccc(-c5cccc6c5oc5ccccc56)cc4)c4ccc(-c5cccc6c5oc5ccccc56)cc4)cc3)cc2)cc1 ; c1ccc(-c2ccc(N(c3ccc(-c4cccc5c4oc4ccccc45)cc3)c3ccc4c5ccccc5c5ccccc5c4c3)cc2)cc1 ; CC1(C)c2ccccc2-c2ccc(N(c3ccc(-c4ccc(-c5ccccc5)cc4)cc3)c3ccc(-c4cccc5c4oc4ccccc45)cc3)cc21]

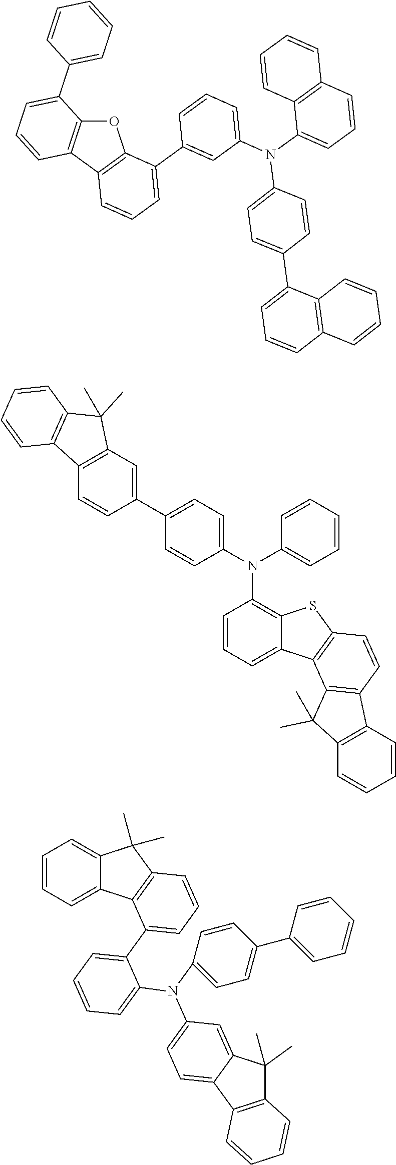 [SMILES: c1ccc(-c2cccc3c2oc2c(-c4cccc(N(c5ccc(-c6cccc7ccccc67)cc5)c5cccc6ccccc56)c4)cccc23)cc1 ; CC1(C)c2ccccc2-c2ccc(-c3ccc(N(c4ccccc4)c4cccc5c4sc4ccc6c(c45)C(C)(C)c4ccccc4-6)cc3)cc21 ; CC1(C)c2ccccc2-c2ccc(N(c3ccc(-c4ccccc4)cc3)c3ccccc3-c3cccc4c3-c3ccccc3C4(C)C)cc21]

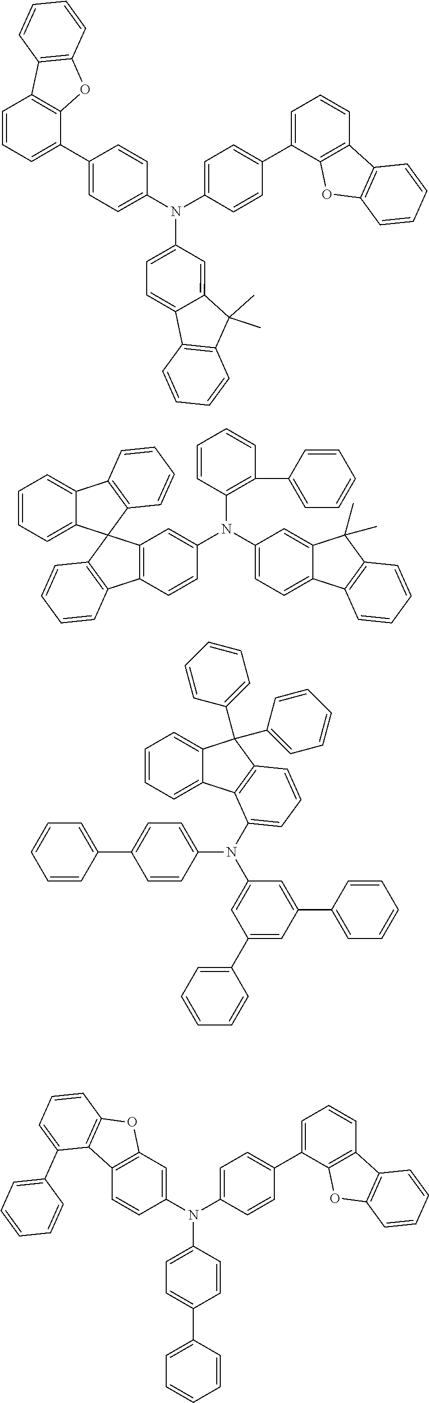

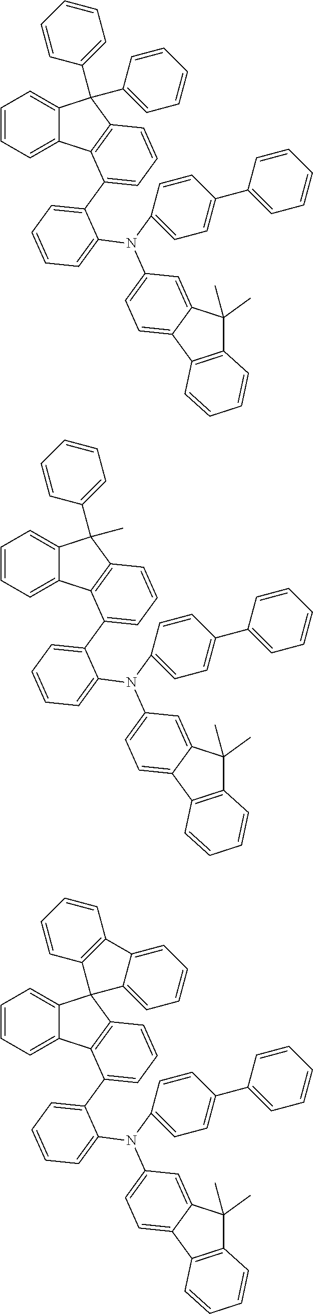 [SMILES: CC1(C)c2ccccc2-c2ccc(N(c3ccc(-c4ccccc4)cc3)c3ccccc3-c3cccc4c3-c3ccccc3C4(c3ccccc3)c3ccccc3)cc21 ; CC1(C)c2ccccc2-c2ccc(N(c3ccc(-c4ccccc4)cc3)c3ccccc3-c3cccc4c3-c3ccccc3C4(C)c3ccccc3)cc21 ; CC1(C)c2ccccc2-c2ccc(N(c3ccc(-c4ccccc4)cc3)c3ccccc3-c3cccc4c3-c3ccccc3C43c4ccccc4-c4ccccc43)cc21]

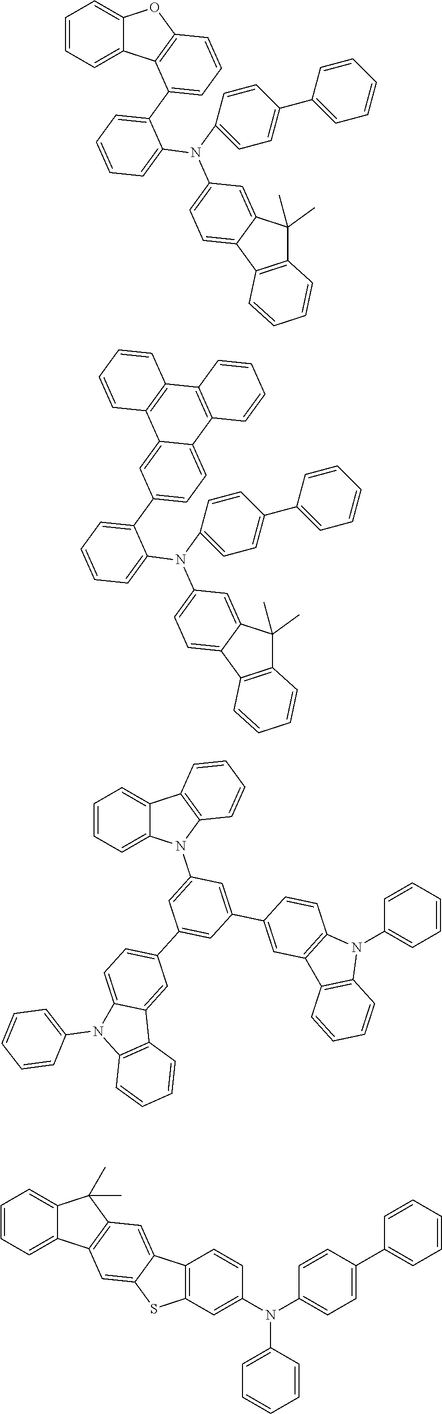

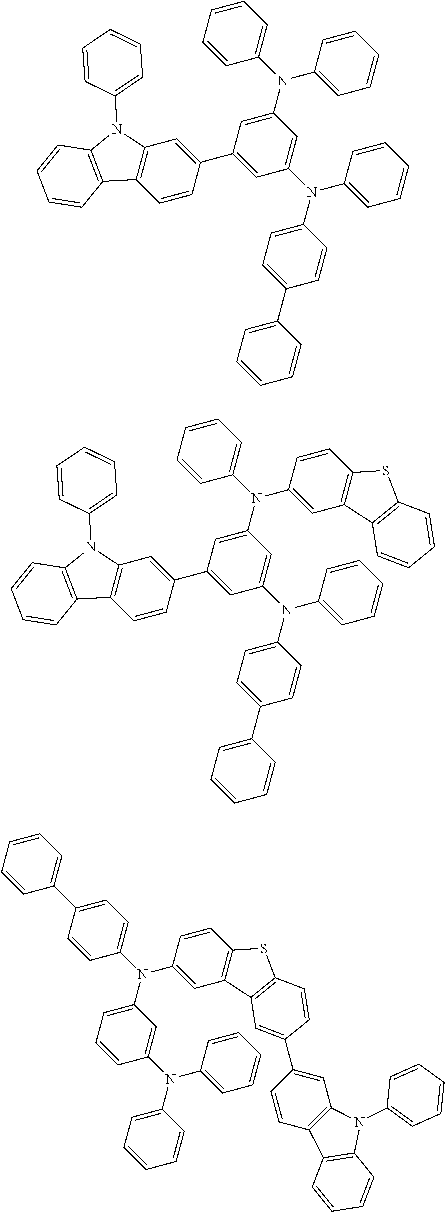 [SMILES: c1ccc(-c2ccc(N(c3ccccc3)c3cc(-c4ccc5c6ccccc6n(-c6ccccc6)c5c4)cc(N(c4ccccc4)c4ccccc4)c3)cc2)cc1 ; c1ccc(-c2ccc(N(c3ccccc3)c3cc(-c4ccc5c6ccccc6n(-c6ccccc6)c5c4)cc(N(c4ccccc4)c4ccc5sc6ccccc6c5c4)c3)cc2)cc1 ; c1ccc(-c2ccc(N(c3cccc(N(c4ccccc4)c4ccccc4)c3)c3ccc4sc5ccc(-c6ccc7c8ccccc8n(-c8ccccc8)c7c6)cc5c4c3)cc2)cc1]

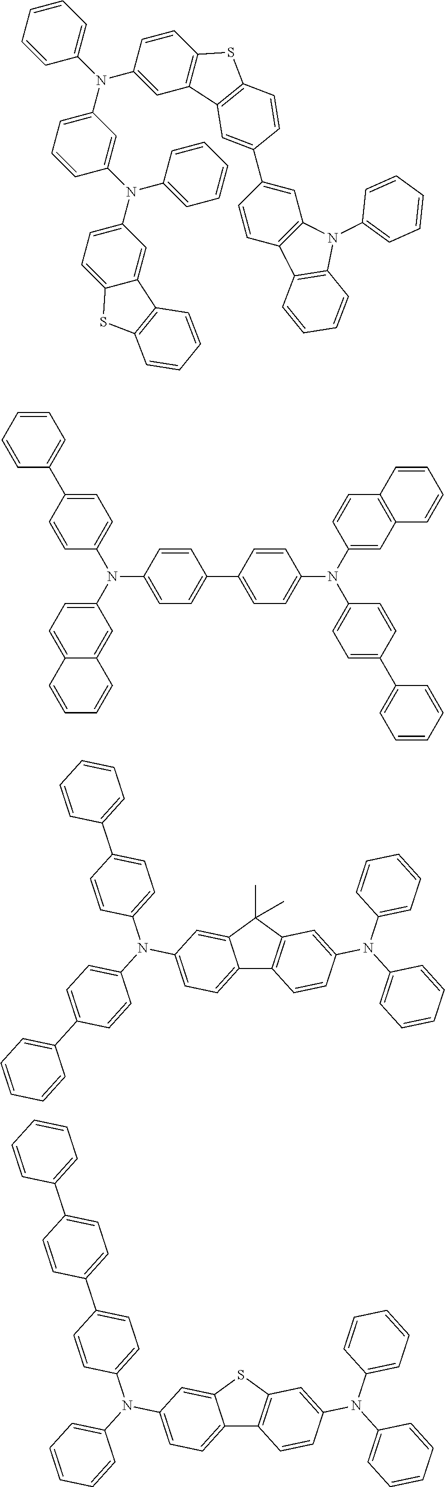

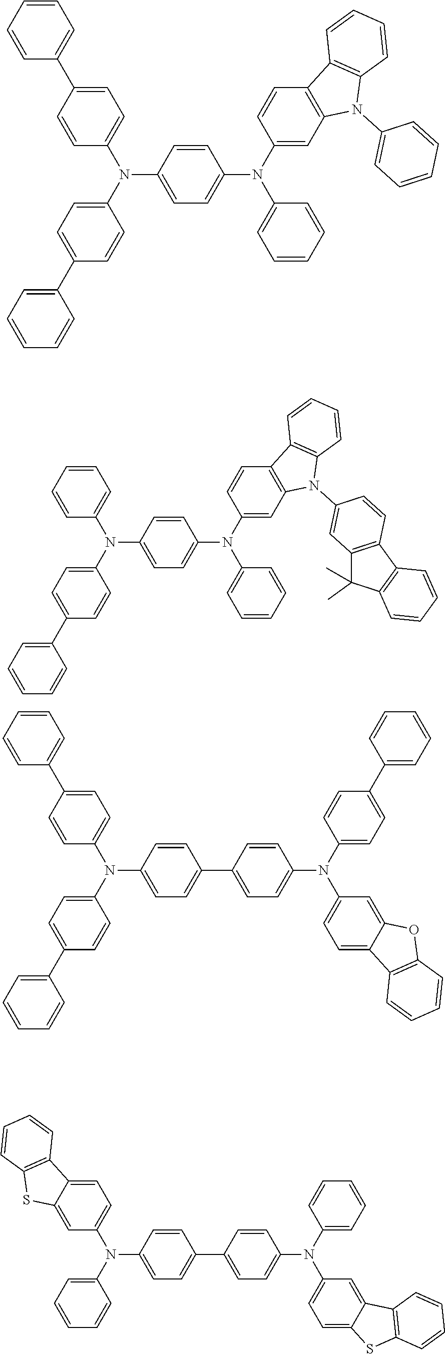

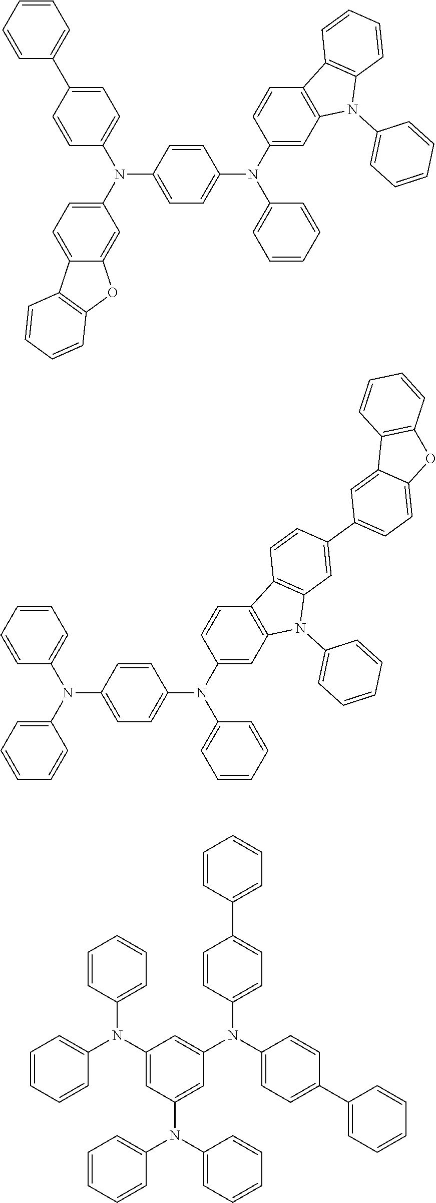 [SMILES: c1ccc(-c2ccc(N(c3ccc(N(c4ccccc4)c4ccc5c6ccccc6n(-c6ccccc6)c5c4)cc3)c3ccc4c(c3)oc3ccccc34)cc2)cc1 ; c1ccc(N(c2ccccc2)c2ccc(N(c3ccccc3)c3ccc4c5ccc(-c6ccc7oc8ccccc8c7c6)cc5n(-c5ccccc5)c4c3)cc2)cc1 ; c1ccc(-c2ccc(N(c3ccc(-c4ccccc4)cc3)c3cc(N(c4ccccc4)c4ccccc4)cc(N(c4ccccc4)c4ccccc4)c3)cc2)cc1]

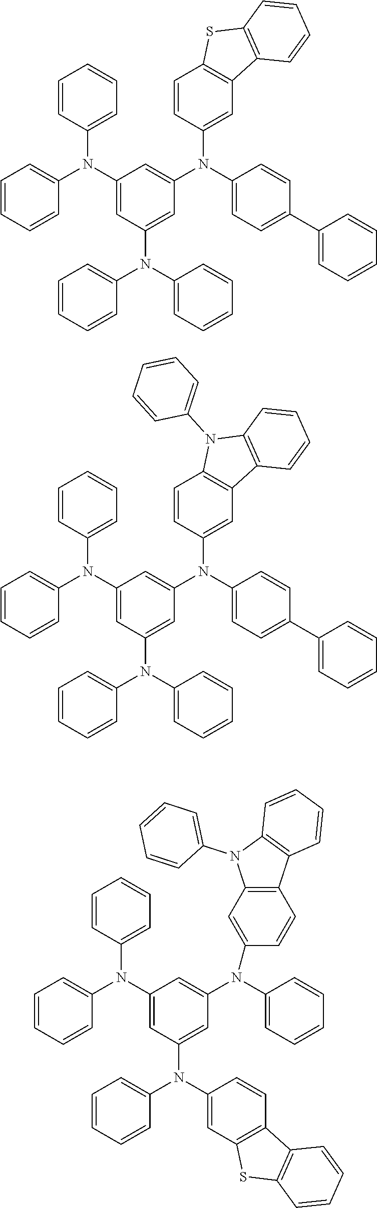

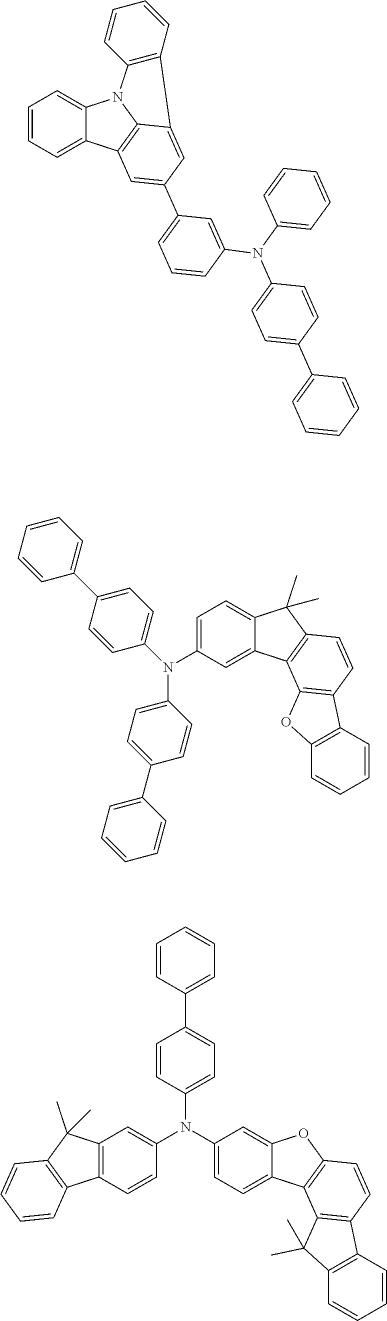 [SMILES: c1ccc(-c2ccc(N(c3ccccc3)c3cccc(-c4cc5c6ccccc6n6c7ccccc7c(c4)c56)c3)cc2)cc1 ; CC1(C)c2ccc(N(c3ccc(-c4ccccc4)cc3)c3ccc(-c4ccccc4)cc3)cc2-c2c1ccc1c2oc2ccccc21 ; CC1(C)c2ccccc2-c2ccc(N(c3ccc(-c4ccccc4)cc3)c3ccc4c(c3)oc3ccc5c(c34)C(C)(C)c3ccccc3-5)cc21]

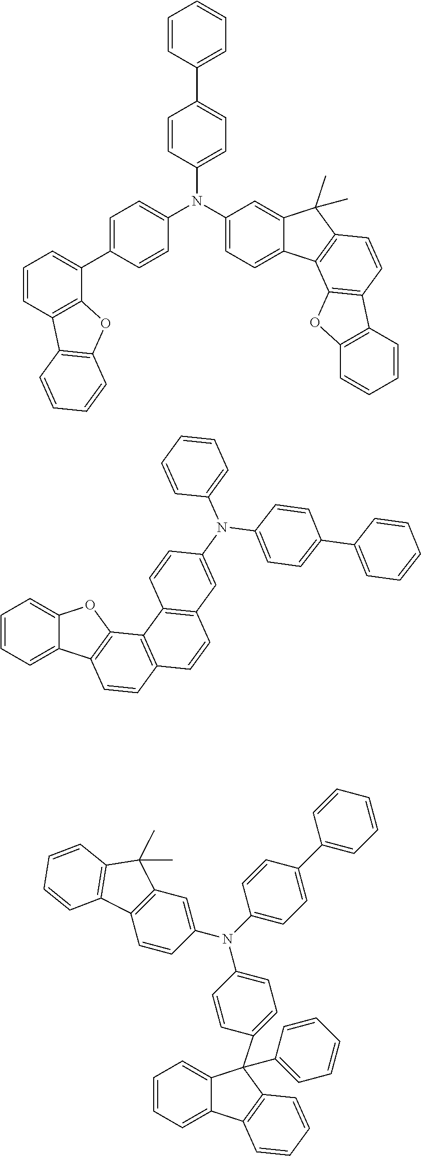 [SMILES: CC1(C)c2cc(N(c3ccc(-c4ccccc4)cc3)c3ccc(-c4cccc5c4oc4ccccc45)cc3)ccc2-c2c1ccc1c2oc2ccccc21 ; c1ccc(-c2ccc(N(c3ccccc3)c3ccc4c(ccc5ccc6c7ccccc7oc6c54)c3)cc2)cc1 ; CC1(C)c2ccccc2-c2ccc(N(c3ccc(-c4ccccc4)cc3)c3ccc(C4(c5ccccc5)c5ccccc5-c5ccccc54)cc3)cc21]

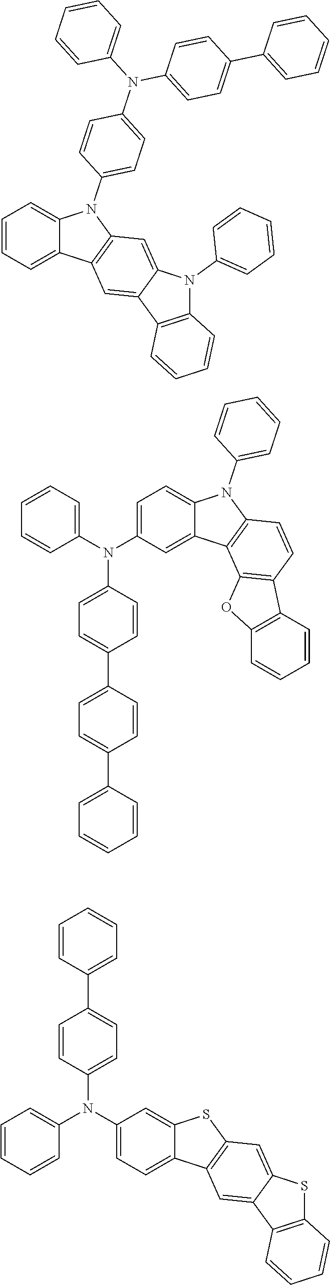

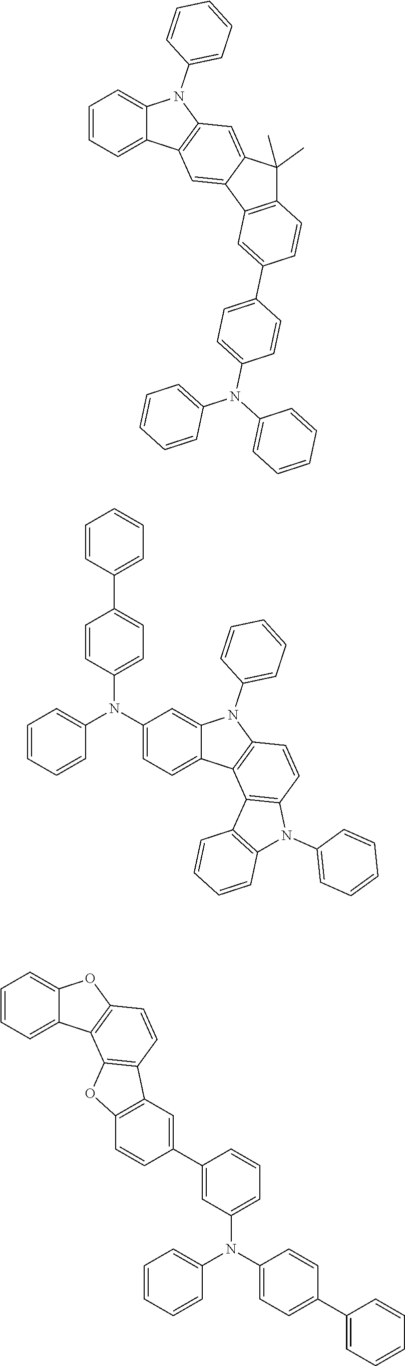 [SMILES: CC1(C)c2ccc(-c3ccc(N(c4ccccc4)c4ccccc4)cc3)cc2-c2cc3c4ccccc4n(-c4ccccc4)c3cc21 ; c1ccc(-c2ccc(N(c3ccccc3)c3ccc4c5c6c7ccccc7n(-c7ccccc7)c6ccc5n(-c5ccccc5)c4c3)cc2)cc1 ; c1ccc(-c2ccc(N(c3ccccc3)c3cccc(-c4ccc5oc6c(ccc7oc8ccccc8c76)c5c4)c3)cc2)cc1]

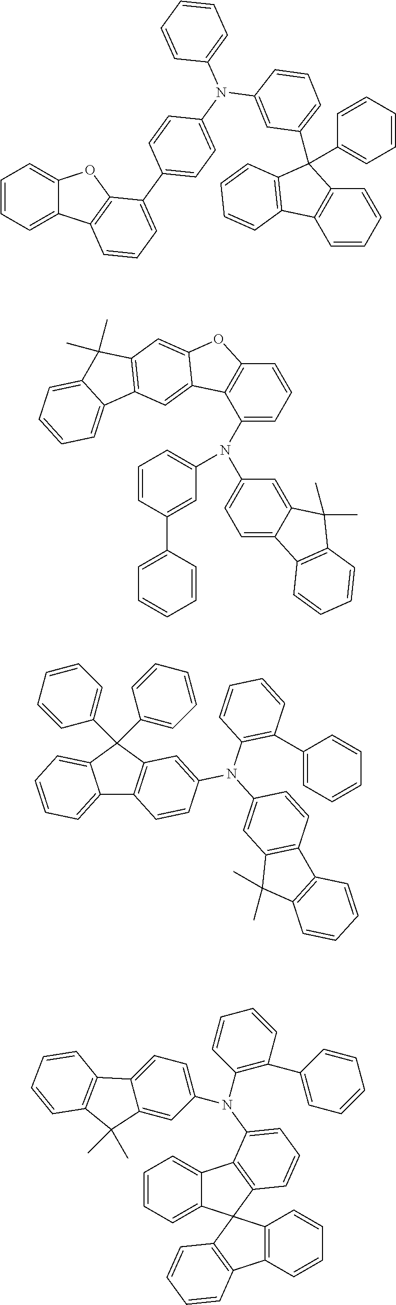 [SMILES: c1ccc(N(c2ccc(-c3cccc4c3oc3ccccc34)cc2)c2cccc(C3(c4ccccc4)c4ccccc4-c4ccccc43)c2)cc1 ; CC1(C)c2ccccc2-c2ccc(N(c3cccc(-c4ccccc4)c3)c3cccc4oc5cc6c(cc5c34)-c3ccccc3C6(C)C)cc21 ; CC1(C)c2ccccc2-c2ccc(N(c3ccc4c(c3)C(c3ccccc3)(c3ccccc3)c3ccccc3-4)c3ccccc3-c3ccccc3)cc21 ; CC1(C)c2ccccc2-c2ccc(N(c3ccccc3-c3ccccc3)c3cccc4c3-c3ccccc3C43c4ccccc4-c4ccccc43)cc21]

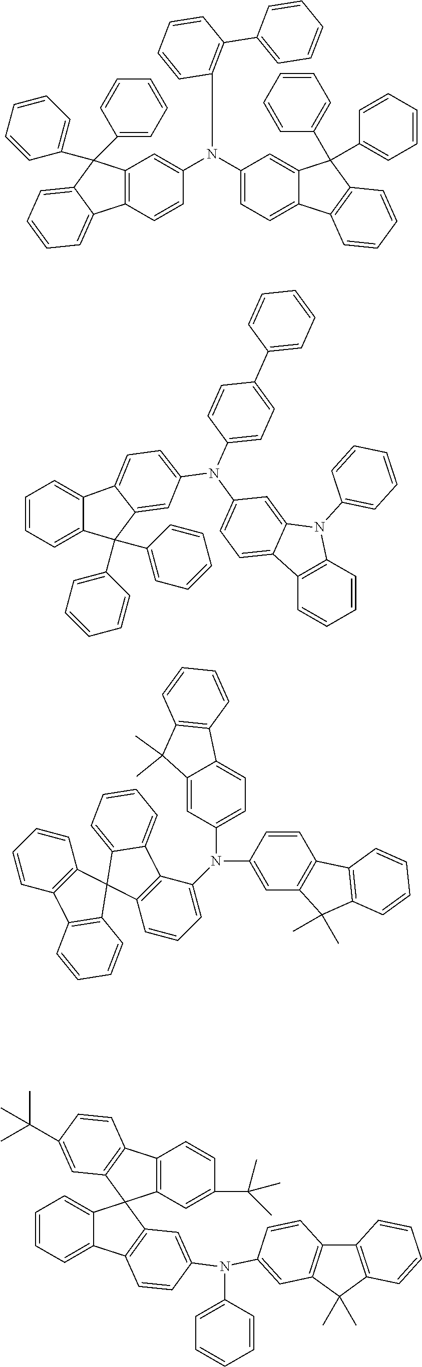 [SMILES: c1ccc(-c2ccccc2N(c2ccc3c(c2)C(c2ccccc2)(c2ccccc2)c2ccccc2-3)c2ccc3c(c2)C(c2ccccc2)(c2ccccc2)c2ccccc2-3)cc1 ; c1ccc(-c2ccc(N(c3ccc4c(c3)C(c3ccccc3)(c3ccccc3)c3ccccc3-4)c3ccc4c5ccccc5n(-c5ccccc5)c4c3)cc2)cc1 ; CC1(C)c2ccccc2-c2ccc(N(c3ccc4c(c3)C(C)(C)c3ccccc3-4)c3cccc4c3-c3ccccc3C43c4ccccc4-c4ccccc43)cc21 ; CC(C)(C)c1ccc2c(c1)C1(c3ccccc3-c3ccc(N(c4ccccc4)c4ccc5c(c4)C(C)(C)c4ccccc4-5)cc31)c1cc(C(C)(C)C)ccc1-2]

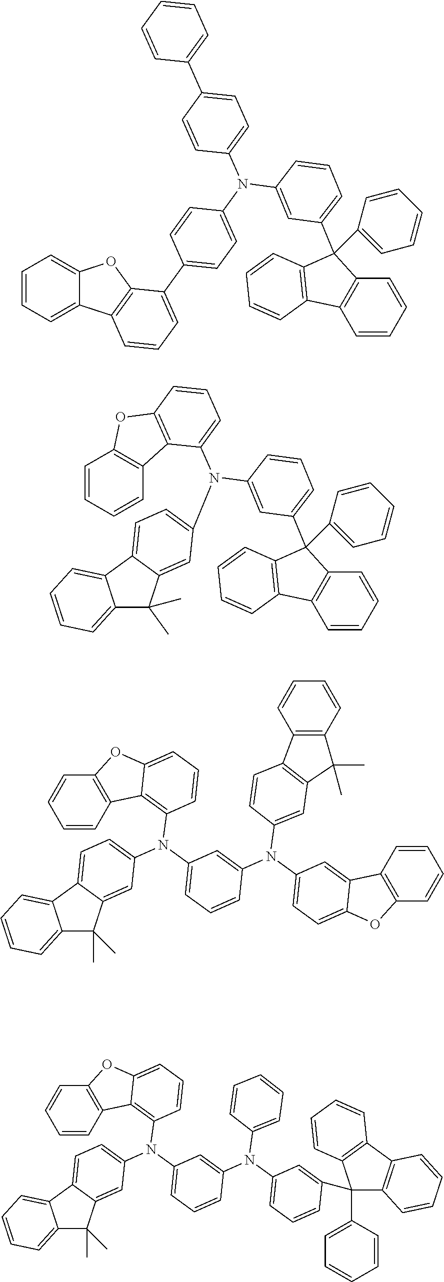

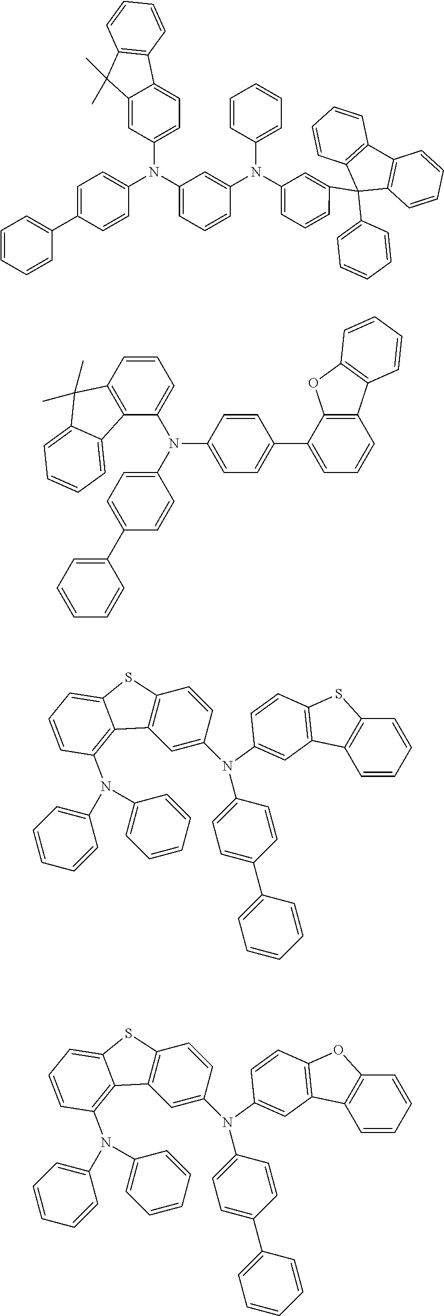

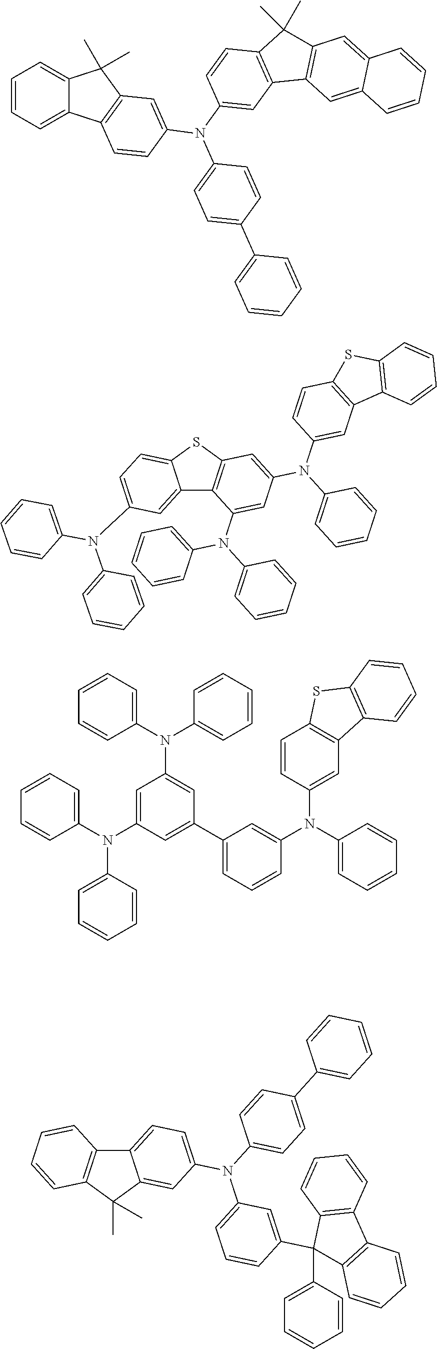 [SMILES: CC1(C)c2ccccc2-c2ccc(N(c3ccc(-c4ccccc4)cc3)c3ccc4c(c3)-c3cc5ccccc5cc3C4(C)C)cc21 ; c1ccc(N(c2cc(N(c3ccccc3)c3ccccc3)c3c(c2)sc2ccc(N(c4ccccc4)c4ccccc4)cc23)c2ccc3sc4ccccc4c3c2)cc1 ; c1ccc(N(c2ccccc2)c2cc(-c3cccc(N(c4ccccc4)c4ccc5sc6ccccc6c5c4)c3)cc(N(c3ccccc3)c3ccccc3)c2)cc1 ; CC1(C)c2ccccc2-c2ccc(N(c3ccc(-c4ccccc4)cc3)c3cccc(C4(c5ccccc5)c5ccccc5-c5ccccc54)c3)cc21]

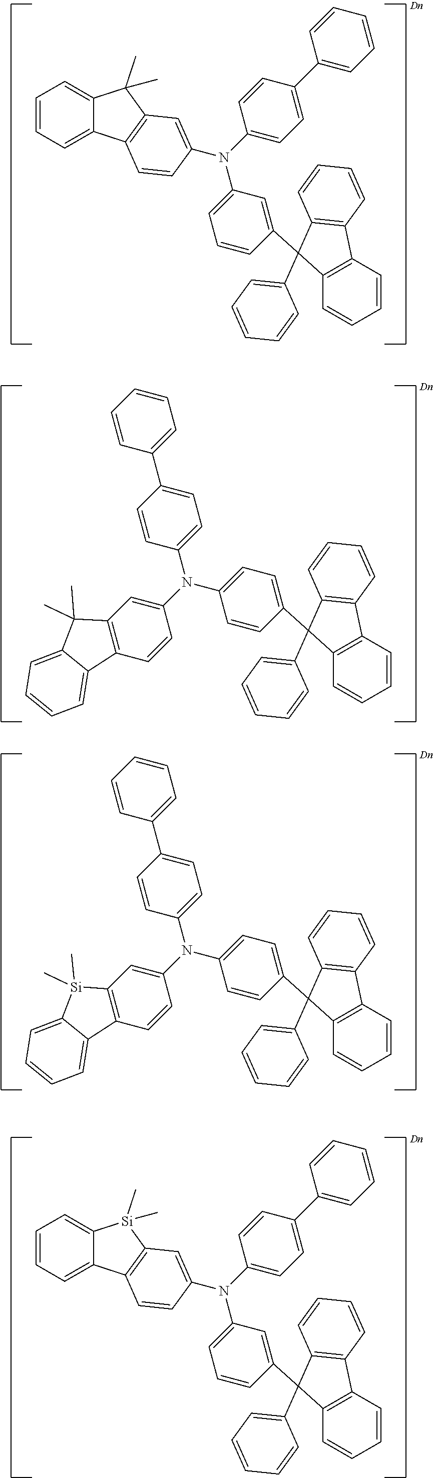

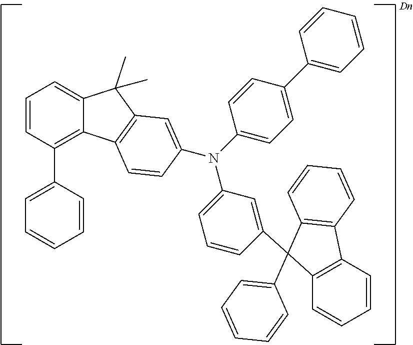

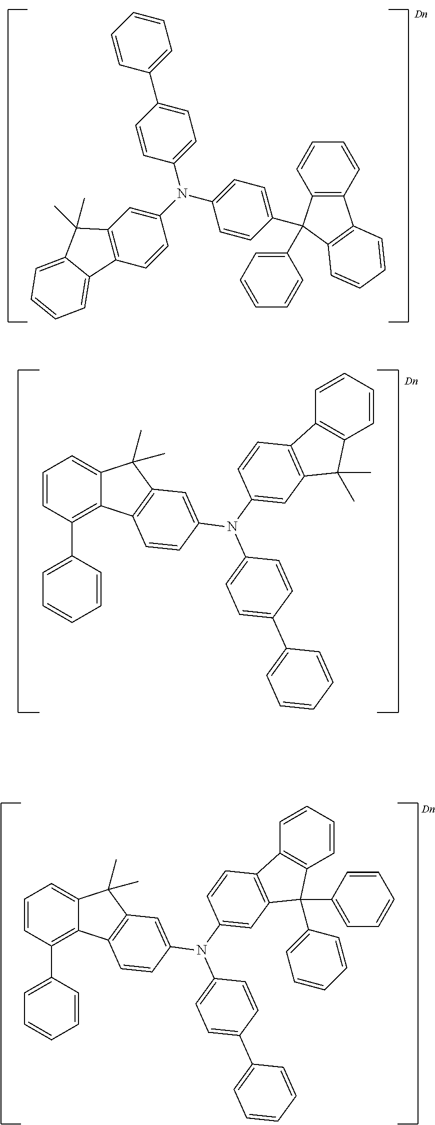

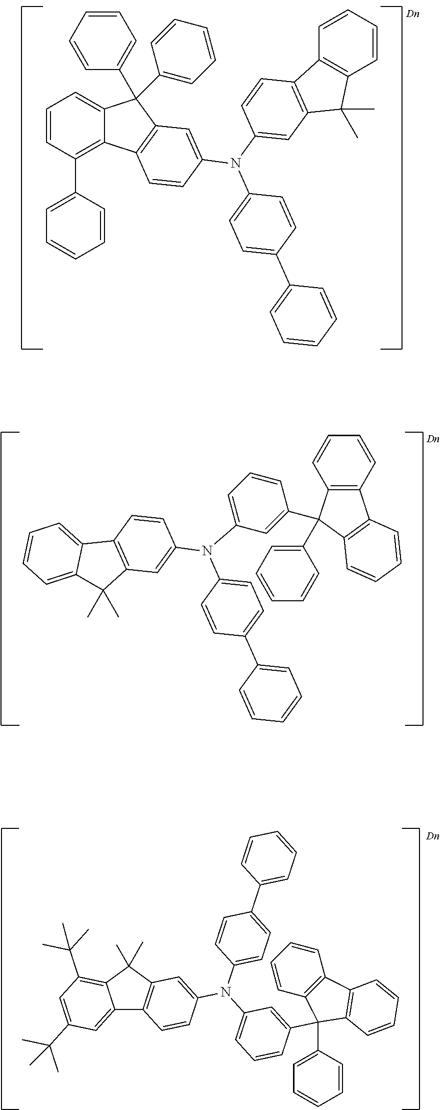

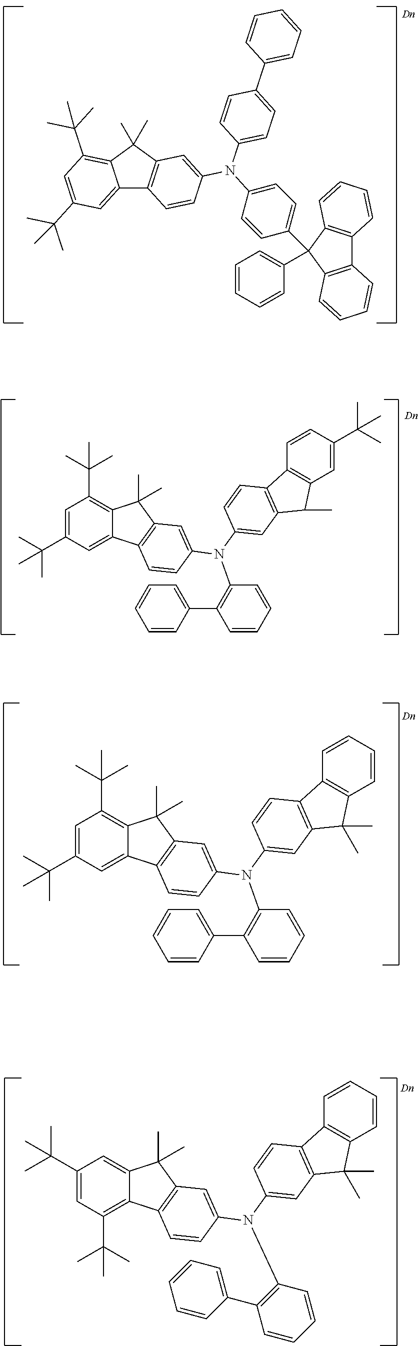

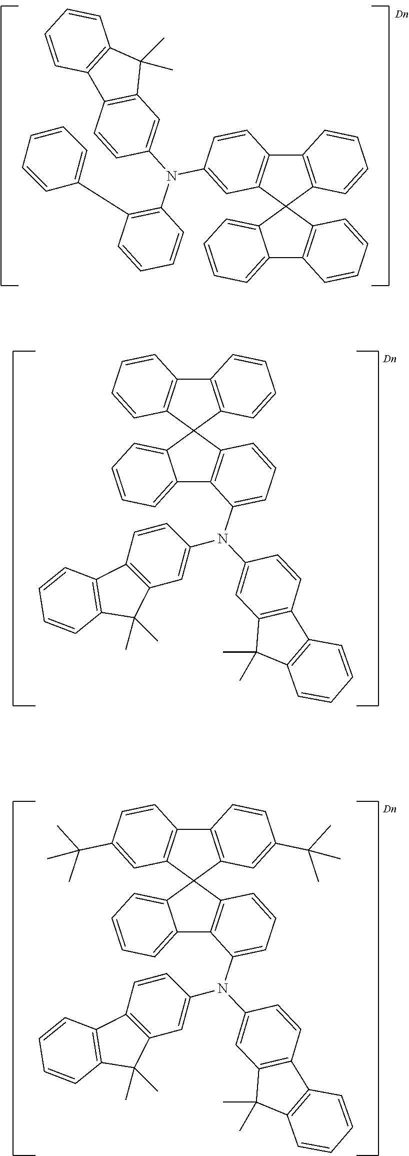

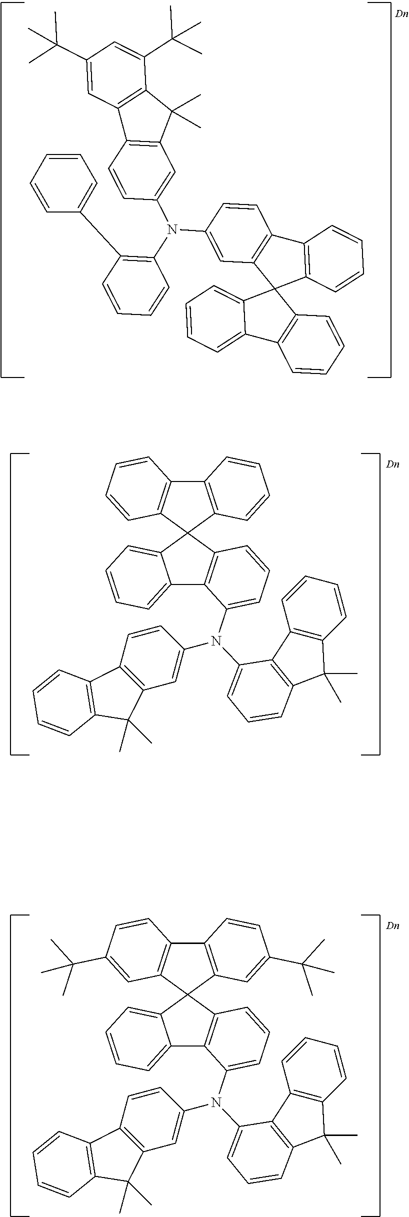

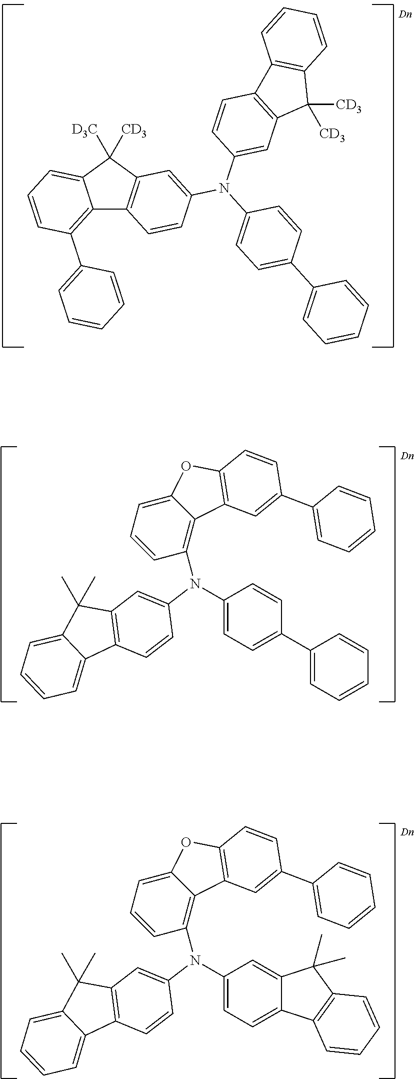

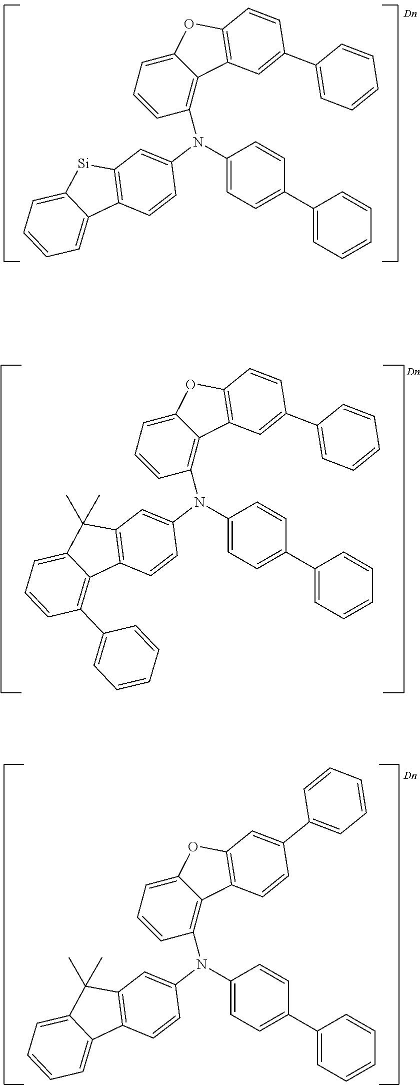

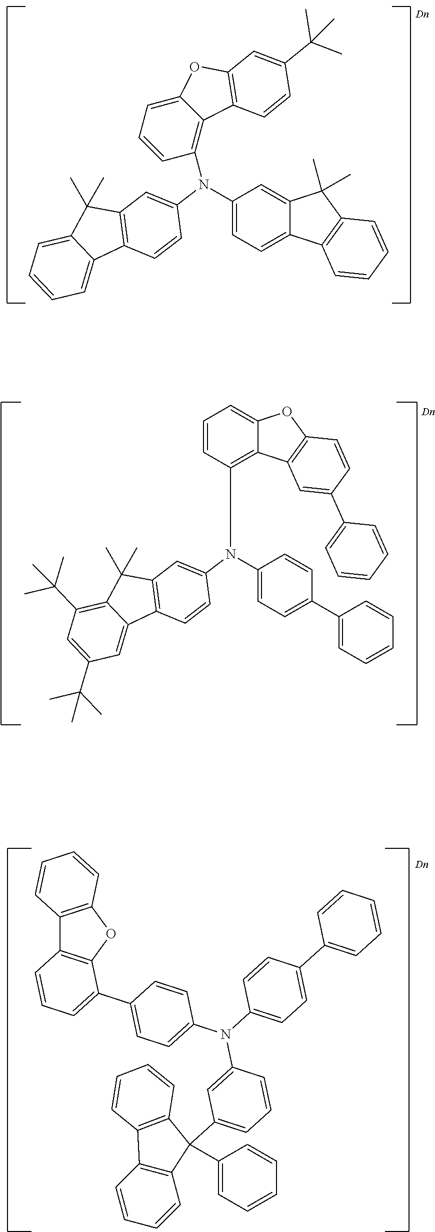

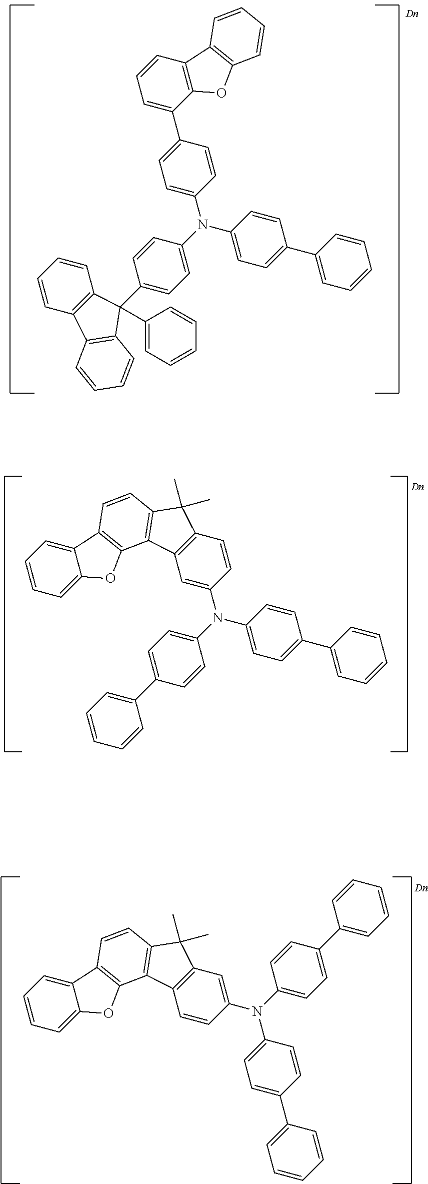

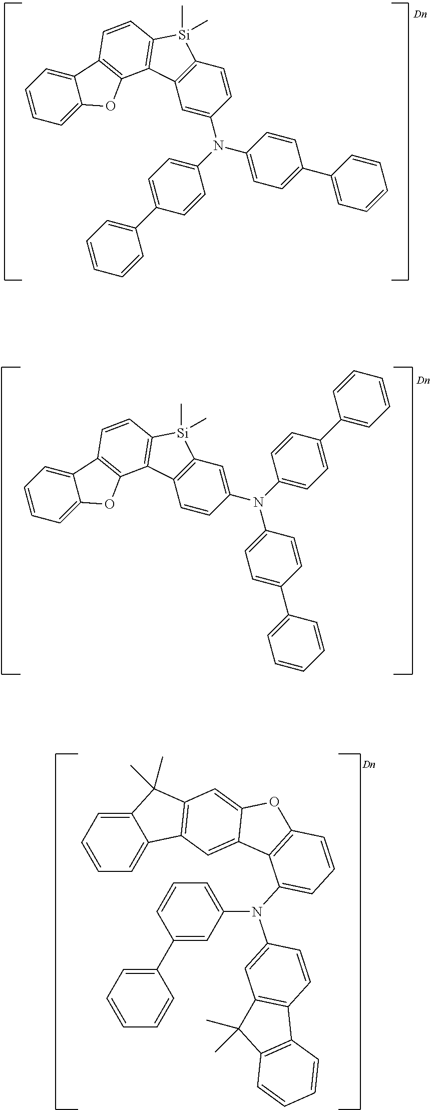

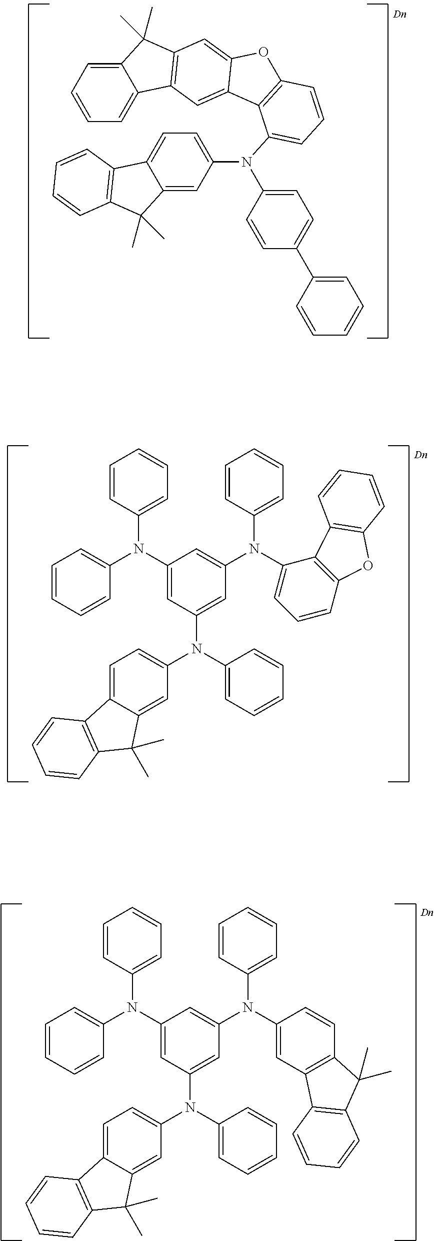

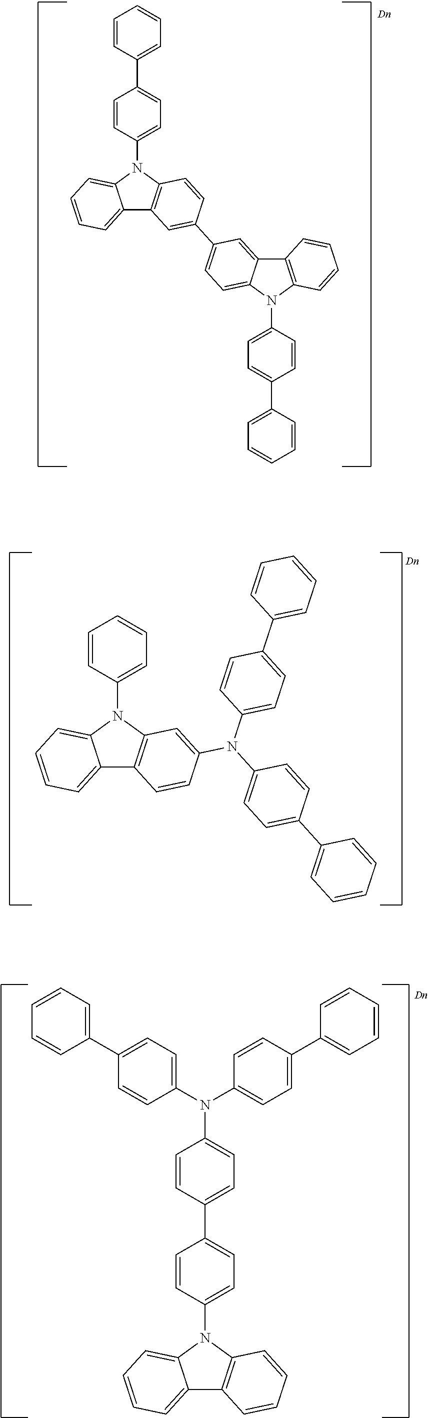

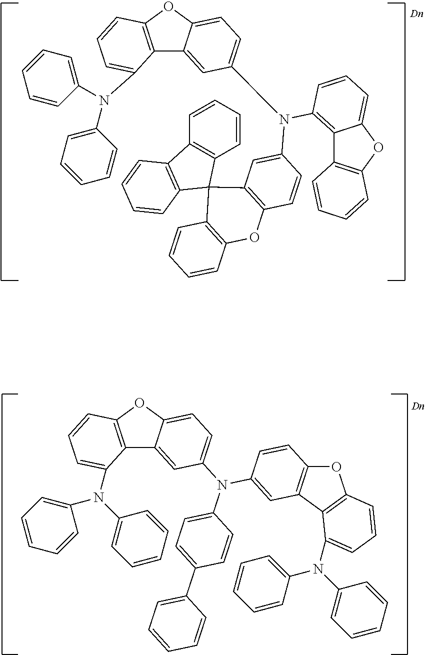

(Dn Refers to the Number of Deuterium Substitutions and Indicates a Structure Substituted with One or More Deuterium Atoms)

In the hole transport region 140, in addition to the compounds described above, other suitable compounds having a similar structure may also be used.

In an implementation, the charge transport region may be, e.g., the electron transport region 150.

The electron transport region 150 may help further increase electron injection or electron mobility and help block holes between the cathode 110 and the light emitting layer 130.

In an implementation, the electron transport region 150 may include an electron transport layer between the cathode 110 and the light emitting layer 130, and an electron transport auxiliary layer between the light emitting layer 130 and the electron transport layer, and a compound of Group C may be included in at least one of the electron transport layer and the electron transport auxiliary layer.

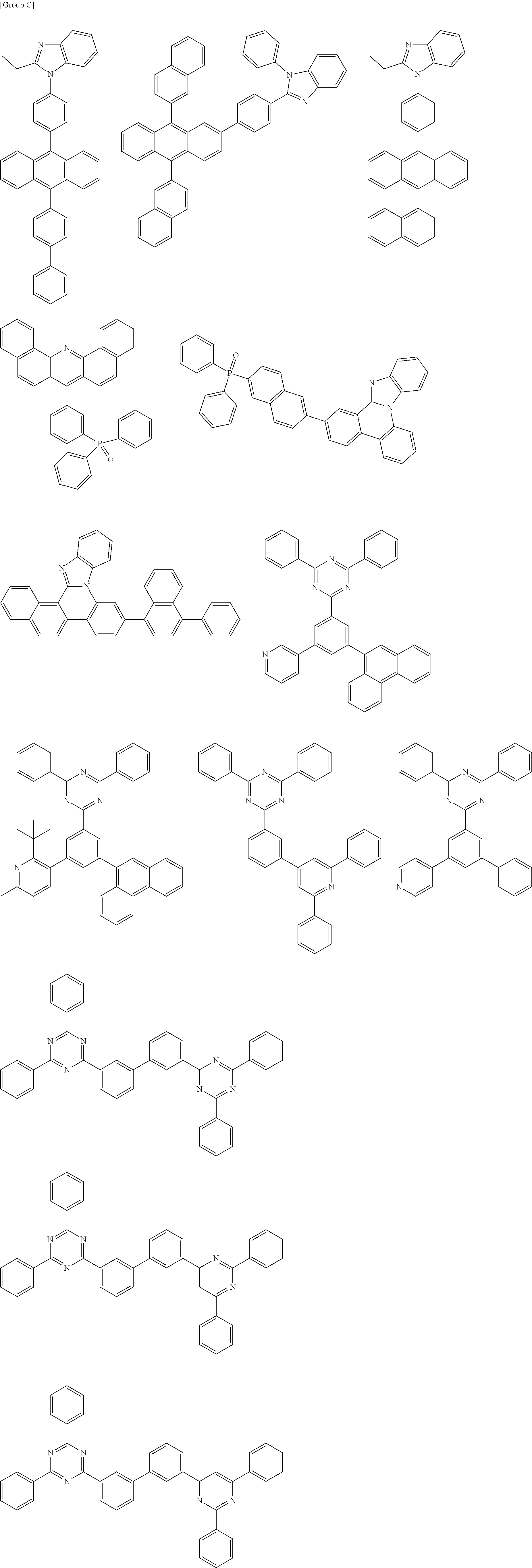

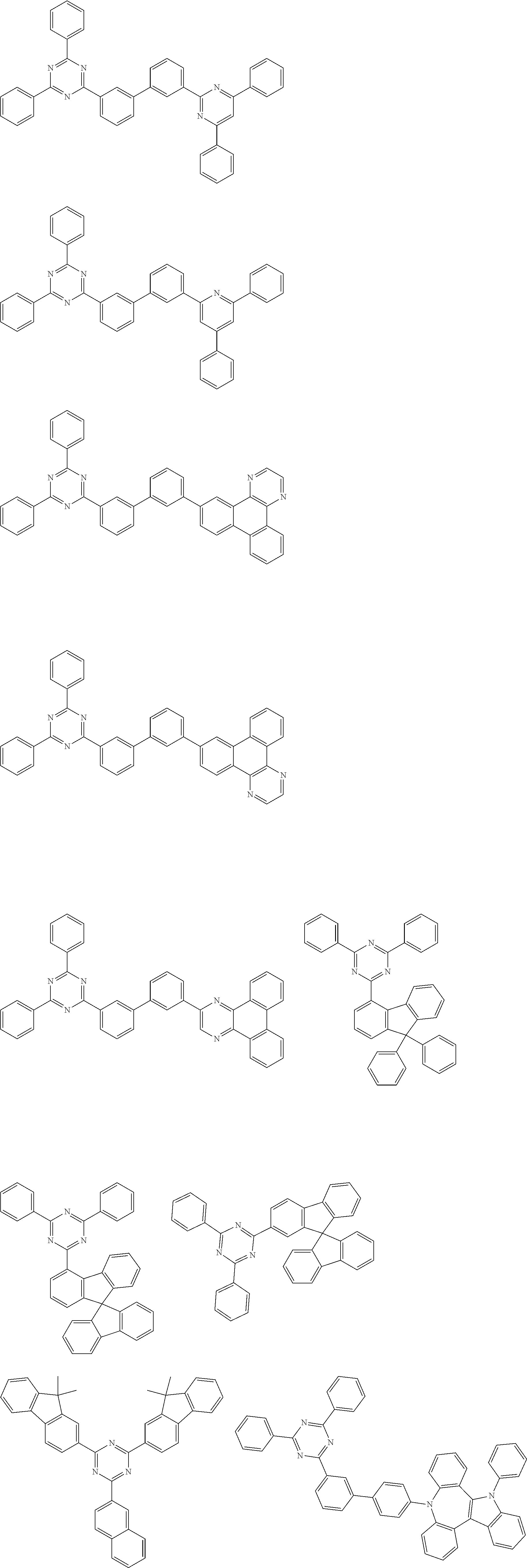

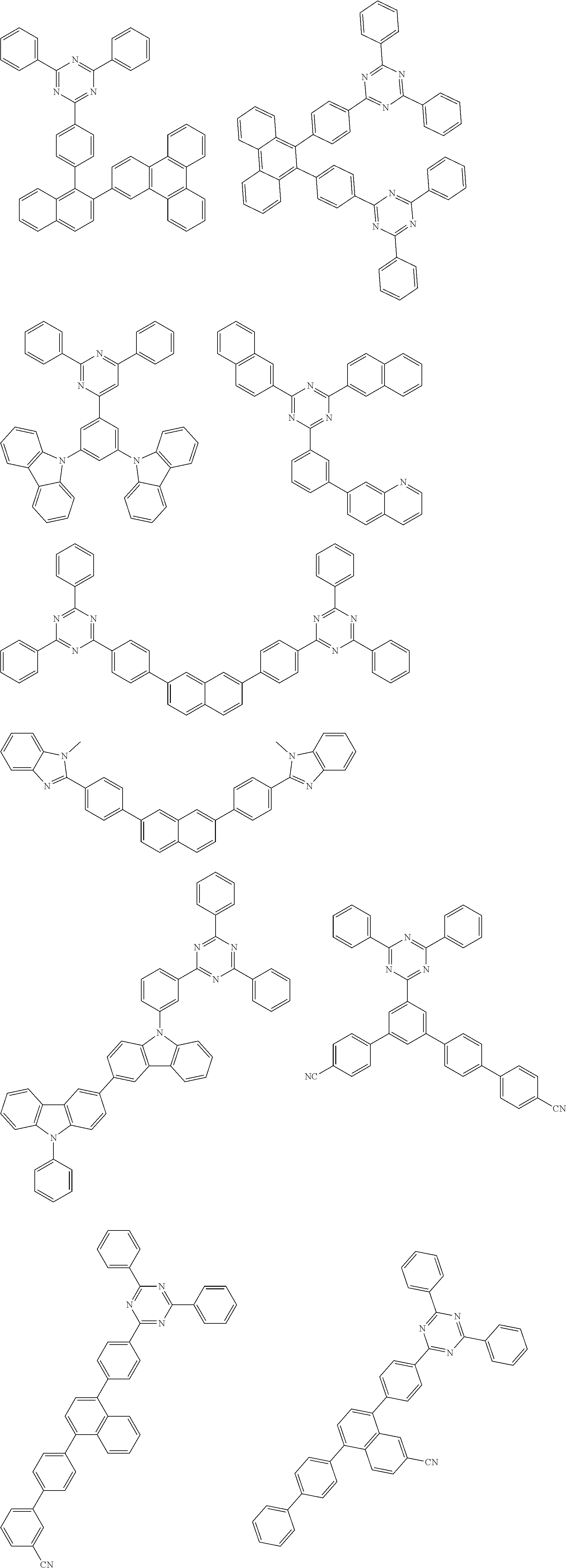

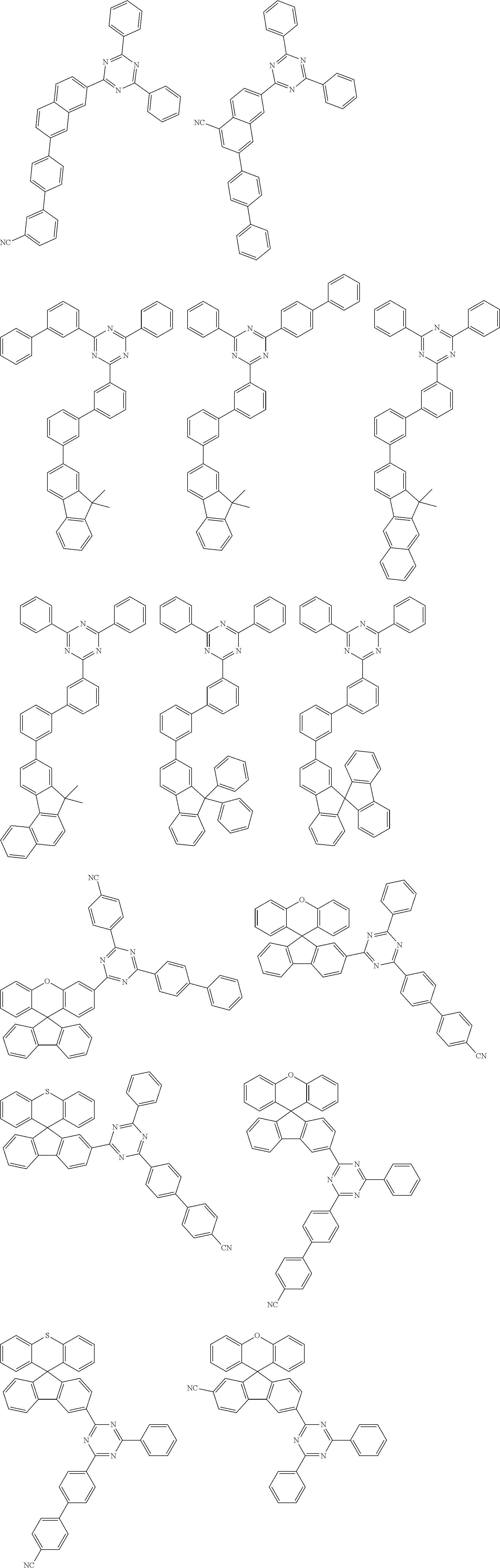

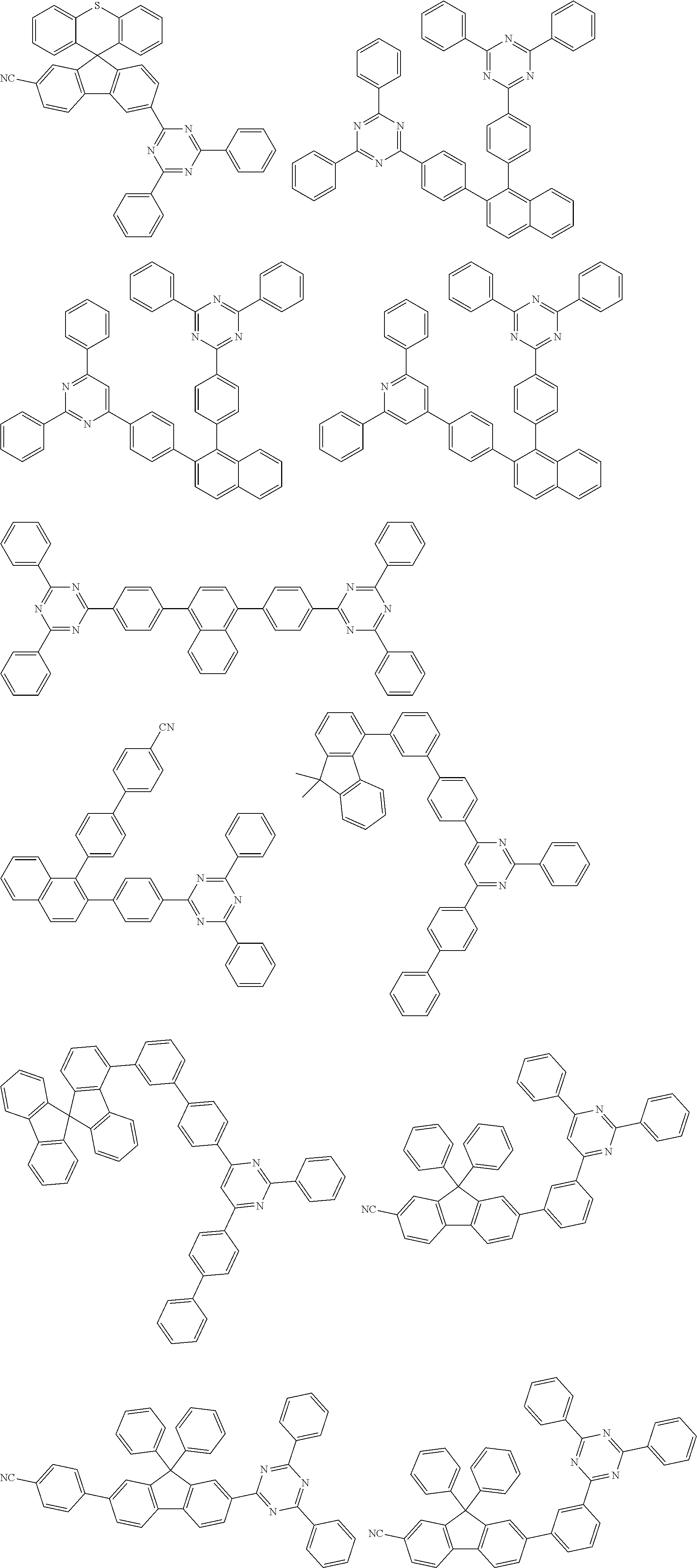

Some example embodiments may be an organic light emitting diode including the light emitting layer as the organic layer.

Some example embodiments may be an organic light emitting diode including a light emitting layer and a hole transport region as the organic layer.

Some example embodiments may be an organic light emitting diode including a light emitting layer and an electron transport region as the organic layer.

An organic light emitting diode according to an implementation may include a hole transport region 140 and an electron transport region 150 in addition to the light emitting layer 130 as the organic layer 105, as shown in the FIGURE.

In an implementation, an organic light emitting diode may further include an electron injection layer, a hole injection layer, or the like, in addition to the light emitting layer as the organic layer.

The organic light emitting diodes 100 may be manufactured by forming an anode or a cathode on a substrate, and then forming an organic layer by a dry film method, e.g., vacuum deposition, sputtering, plasma plating or ion plating, and forming a cathode or an anode thereon.

The organic light emitting diode may be applied to an organic light emitting display device.

The following Examples and Comparative Examples are provided in order to highlight characteristics of one or more embodiments, but it will be understood that the Examples and Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples.

Hereinafter, starting materials and reactants used in Examples and Synthesis Examples were purchased from Sigma-Aldrich Co. Ltd., TCI Inc., Tokyo chemical industry, or P&H tech as far as there is no particular comment or were synthesized by suitable methods.

### Synthesis of Compound for Organic Optoelectronic Device

### Synthesis Example 1: Synthesis of Compound A-2

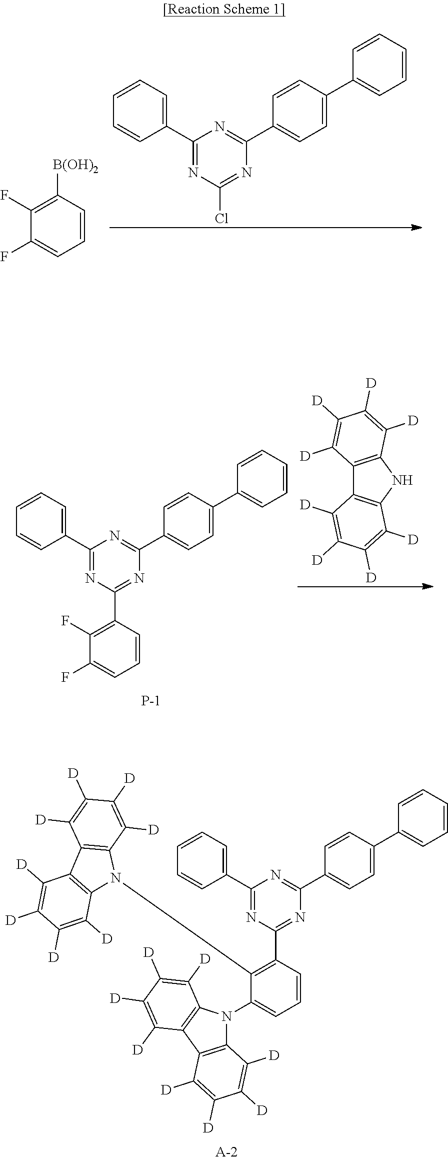

### 1st Step: Synthesis of Intermediate P-1

2-([1,1′-biphenyl]-4-yl)-4-chloro-6-phenyl-1,3,5-triazine (30 g/1.0 eq.), (2,3-difluorophenyl)boronic acid (1.1 eq.), Pd(PPh₃)₄(0.05 eq.), and K₂CO₃ (3.0 eq.) were added to a flask together with THE (tetrahydrofuran) (750 mL) and distilled water (250 mL) and then, refluxed at 80° C. After 12 hours, a reaction was terminated, and the resultant was diluted with DCM (dichloromethane), three times washed with brine, and dried with MgSO₄. Subsequently, column chromatography was performed to obtain 25 g of Intermediate P-1.

### 2nd Step: Synthesis of Compound A-2

Intermediate P-1 (10 g/1.0 eq.), 9H-carbazole-d8 (2.5 eq.), and K₃PO₄ (3.0 eq.) along with DMF (dimethylformamide) (200 mL) were added to the flask and then, refluxed at 150° C. After 12 hours, a reaction was terminated, and the reaction solution was diluted with DCM, three times washed with brine, and dried with MgSO₄. 8 g of Compound A-2 was obtained through column chromatography.

### Synthesis Example 2: Synthesis of Compound A-8

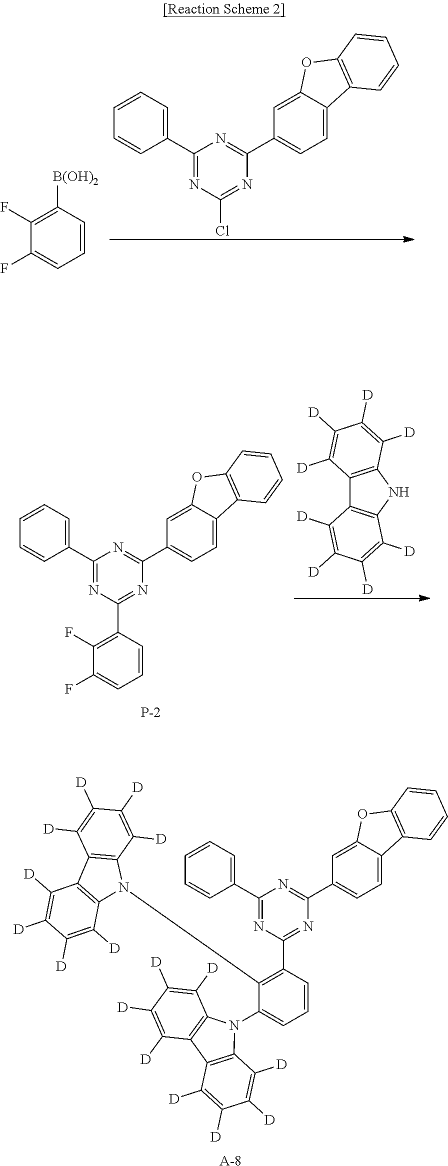

### 1 st step: Synthesis of Intermediate P-2

2-chloro-4-(dibenzo[b,d]furan-3-yl)-6-phenyl-1,3,5-triazine (30 g/1.0 eq.), (2,3-difluorophenyl)boronic acid (1.1 eq.), Pd(PPh₃)₄(0.05 eq.), and K₂CO₃ (3.0 eq.) were added to a flask together with THE (750 mL) and distilled water (250 mL) and then, refluxed at 80° C. After 12 hours, a reaction was terminated, and the resultant was diluted with DCM, three times washed with brine, and dried with MgSO₄. Subsequently, column chromatography was performed to obtain 22 g of Intermediate P-2.

### 2nd step: Synthesis of Compound A-8

Intermediate P-2 (10 g/1.0 eq.), 9H-carbazole-d8 (2.5 eq.), and K₃PO₄ (3.0 eq.) were added to a flask together with DMF (200 mL) and then, refluxed at 150° C. After 12 hours, a reaction was terminated, and the resultant was diluted with DCM, three times washed with brine, and dried with MgSO₄. Subsequently, column chromatography was performed to obtain 7 g of Compound A-8.

### Synthesis Example 3: Synthesis of Compound A-9

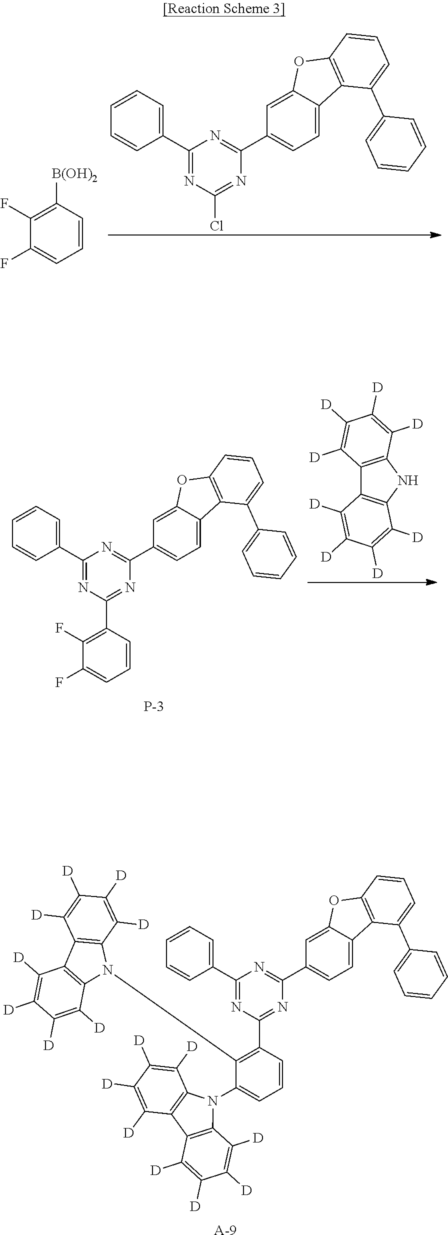

### 1st step: Synthesis of Intermediate P-3

2-chloro-4-phenyl-6-(9-phenyldibenzo[b,d]furan-3-yl)-1,3,5-triazine (30 g/1.0 eq.), (2,3-difluorophenyl)boronic acid (1.1 eq.), Pd(PPh₃)₄(0.05 eq.), and K₂CO₃ (3.0 eq.) were added to a flask together with THE (750 mL) and distilled water (250 mL) and then, refluxed at 80° C. After 12 hours, a reaction was terminated, and the resultant was diluted with DCM, three times washed with brine, and dried with MgSO₄. Subsequently, column chromatography was performed to obtain 26 g of Intermediate P-3.

### 2nd step: Synthesis of Compound A-9

Intermediate P-3 (10 g/1.0 eq.), 9H-carbazole-d8 (2.5 eq.), and K₃PO₄ (3.0 eq.) were added to a flask together with DMF (200 mL) and then, refluxed at 150° C. After 12 hours, a reaction was terminated, and the resultant was diluted with DCM, three times washed with brine, and dried with MgSO₄. Subsequently, column chromatography was performed to obtain 8 g of Compound A-9.

### Synthesis Example 4: Synthesis of Compound C-4

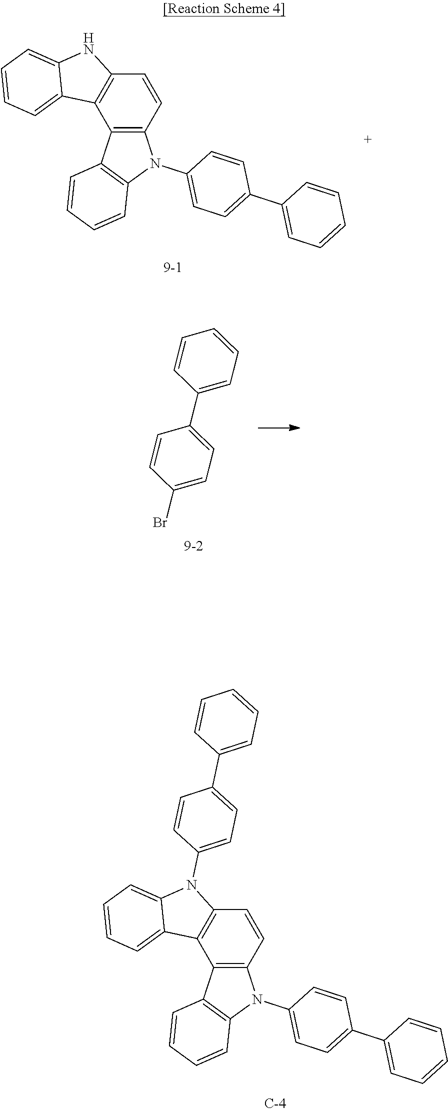 [SMILES: c1ccc(-c2ccc(-n3c4ccccc4c4c5c(ccc43)[nH]c3ccccc35)cc2)cc1 ; Brc1ccc(-c2ccccc2)cc1 ; c1ccc(-c2ccc(-n3c4ccccc4c4c5c6ccccc6n(-c6ccc(-c7ccccc7)cc6)c5ccc43)cc2)cc1]

10.0 g (24.5 mmol) of Intermediate 9-1, 6.3 g (26.9 mmol) of Intermediate 9-2, 1.1 g (1.2 mmol) of Pd₂(dba)₃, 3.5 g (36.7 mmol) of NaOtBu, and 0.7 g (3.7 mmol) of P(t-Bu)₃ were added to a round-bottomed flask and after adding xylene (122 ml) thereto, and stirred under reflux at 140° C. for 8 hours. When a reaction was completed, distilled water was added thereto and then, stirred, and after removing an aqueous layer, an organic layer therefrom was filtered through a silica gel to obtain 10.3 g (75%) of Compound C-4.

(LC/MS theoretical value: 560.23 g/mol, measured value: M+=561.54 g/mol)

### Synthesis Example 5: Synthesis of Compound B-136

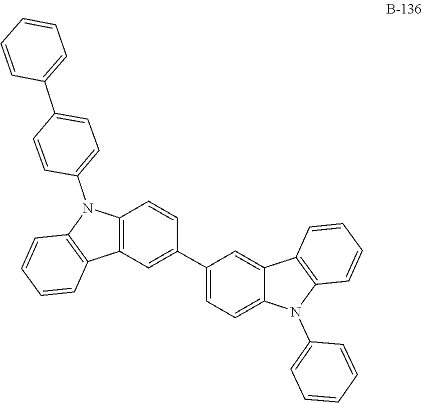 [SMILES: c1ccc(-c2ccc(-n3c4ccccc4c4cc(-c5ccc6c(c5)c5ccccc5n6-c5ccccc5)ccc43)cc2)cc1]

Compound B-136 was obtained by purchase from GemChem.

HRMS (70 eV, EI+): m/z calcd for C42H28N2: 560.2252, found: 560.

Elemental Analysis: C, 90%; H, 5%

### Synthesis Example 6: Synthesis of Compound R-1

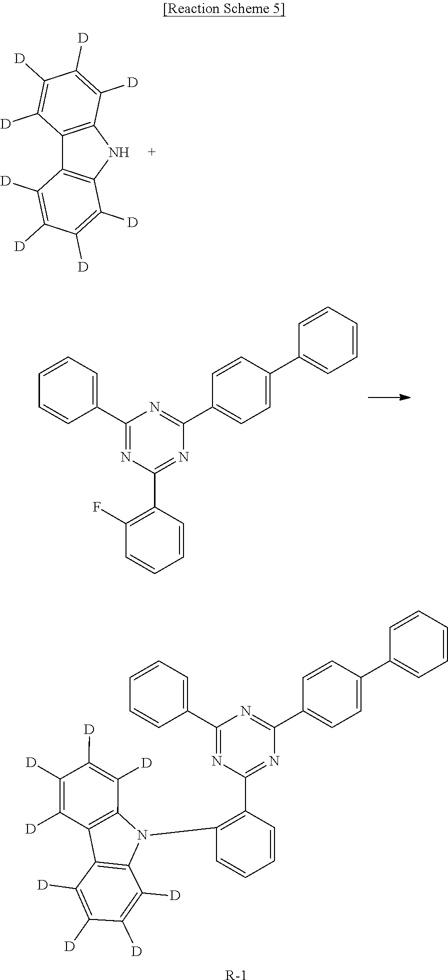 [SMILES: [2H]c1c([2H])c([2H])c2c([nH]c3c([2H])c([2H])c([2H])c([2H])c32)c1[2H] ; Fc1ccccc1-c1nc(-c2ccccc2)nc(-c2ccc(-c3ccccc3)cc2)n1 ; [2H]c1c([2H])c([2H])c2c(c1[2H])c1c([2H])c([2H])c([2H])c([2H])c1n2-c1ccccc1-c1nc(-c2ccccc2)nc(-c2ccc(-c3ccccc3)cc2)n1 ; "R-1"]

2-([1,1′-biphenyl]-4-yl)-4-(2-fluorophenyl)-6-phenyl-1,3,5-triazine (10 g/1.0 eq.), 9H-carbazole-d8 (1.5 eq.), and K₃PO₄ (3.0 eq.) were added to a flask together with DMF (200 mL) and then, refluxed at 150° C. After 12 hours, a reaction was terminated, and the resultant was diluted with DCM, three times washed with brine, and dried with MgSO₄. Subsequently, column chromatography was performed to obtain 6 g of Compound R-1.

### Synthesis Example 7: Synthesis of Compound R-2

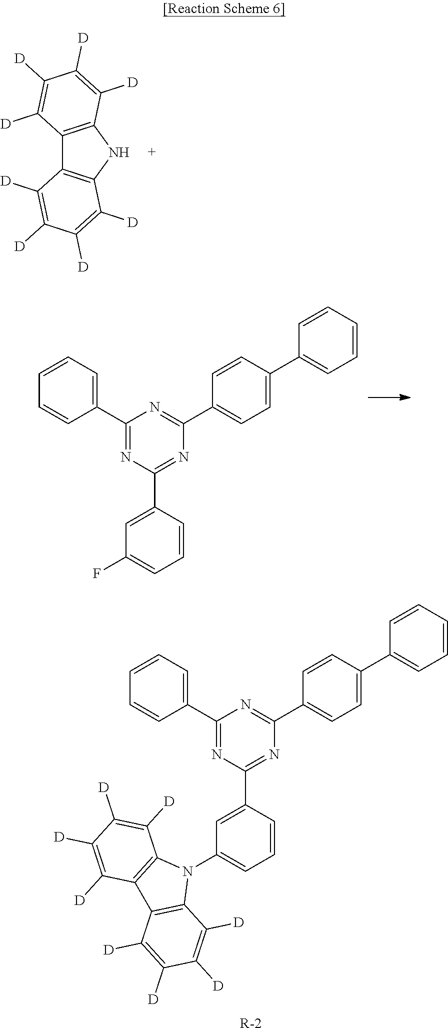

2-([1,1′-biphenyl]-4-yl)-4-(3-fluorophenyl)-6-phenyl-1,3,5-triazine (10 g/1.0 eq.), 9H-carbazole-d8 (1.5 eq.), and K₃PO₄ (3.0 eq.) were added to a flask together with DMF (200 mL) and then, refluxed at 150° C. After 12 hours, a reaction was terminated, and the resultant was diluted with DCM, three times washed with brine, and dried with MgSO₄. Subsequently, column chromatography was performed to obtain 7 g of Compound R-2.

### Synthesis Example 8: Synthesis of Compound R-3

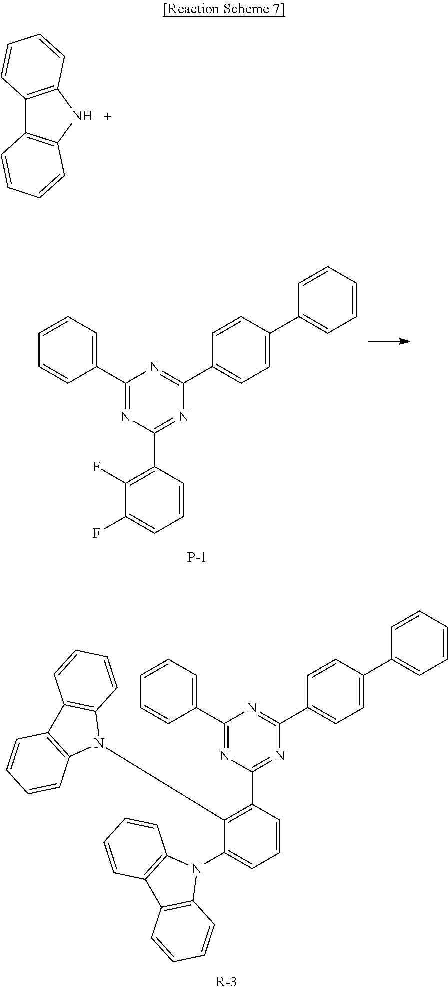

Intermediate P-1 (10 g/1.0 eq.), 9H-carbazole (2.5 eq.), and K₃PO₄ (3.0 eq.) along with DMF (200 mL) were added to a flask and then, refluxed at 150° C. After 12 hours, a reaction was terminated, and the resultant was diluted with DCM, three times washed with brine, and dried with MgSO₄. Subsequently, column chromatography was performed to obtain 7 g of Compound R-3.

### Example 1: Manufacturing of Green Organic Light Emitting Diode

A glass substrate coated with a thin film of ITO (indium tin oxide) was ultrasonically cleaned with distilled water. After washing with the distilled water, the glass substrate was ultrasonically washed with isopropyl alcohol, acetone, or methanol, and dried and then, moved to a plasma cleaner, cleaned by using oxygen plasma for 10 minutes, and moved to a vacuum depositor. This prepared ITO transparent electrode was used as an anode, Compound E doped with 3% NDP-9 (Novaled GmbH) was vacuum-deposited on the ITO substrate to form a 100 Å-thick hole injection layer, and Compound E was deposited on the hole injection layer to a thickness of 1,350 Å to form a hole transport layer. Compound F was deposited on the hole transport layer to a thickness of 320 Å to form a hole transport auxiliary layer. On the hole transport auxiliary layer, Compound A-2 and Compound C-4 were simultaneously used as hosts in a weight ratio of 4:6, and PhGD was doped at 10 wt % as a dopant to form a 380 Å-thick light emitting layer by vacuum deposition. Subsequently, Compound G was deposited on the light emitting layer to a thickness of 50 Å to form an electron transport auxiliary layer, and Compound H and LiQ were simultaneously vacuum-deposited in a weight ratio of 1:1 to form a 300 Å-thick electron transport layer. An organic light emitting diode was manufactured by sequentially vacuum-depositing 15 Å of LiQ and 1,200 Å of Al on the electron transport layer to form a cathode.

The organic light emitting diode was manufactured to have a structure of ITO/Compound E (3% NDP-9 doping, 100 Å)/Compound E (1,350 Å)/Compound F (320 Å)/EML [Host (Compound A-2: Compound C-4=4: 6 wt %/wt %): PhGD=90 wt %: 10 wt %](380 Å)/Compound G (50 Å)/Compound H:LiQ (300 Å)/LiQ (15 Å)/Al (1,200 Å).
Compound E: N-(9,9-diphenyl-9H-fluoren-2-yl)-N,9-diphenyl-9H-carbazol-2-amine
Compound F: 9,9-dimethyl-N-[3-(9-phenyl-9H-fluoren-9-yl)phenyl]-4-(4-phenylphenyl)-9H-fluoren-2-amine
Compound G: 4-{4-[4-(9,9-dimethyl-9H-fluoren-4-yl)phenyl]phenyl}-2-phenyl-6-(4-phenylphenyl)pyrimidine
Compound H: 2-(4-{1-[4-(diphenyl-1,3,5-triazin-2-yl)phenyl]naphthalene-2-yl}-4,6-diphenyl-1,3,5-triazine

### Examples 2 and 3 and Comparative Examples 1 to 3

Each organic light emitting diode was manufactured in the same manner as Example 1, except that the compositions were changed to those shown in Table 1.

### Evaluation

The driving voltage, luminous efficiency, and life-span characteristics of the organic light emitting diodes according to Examples 1 to 3 and Comparative Examples 1 to 3 were evaluated.

The specific measurement method is as follows, and the results are as shown in Table 1.

### (1) Measurement of Current Density Change Depending on Voltage Change

The obtained organic light emitting diodes were measured regarding a current value flowing in the unit device, while increasing the voltage from 0 V to 10 V using a current-voltage meter (Keithley 2400), and the measured current value was divided by area to provide the results.

### (2) Measurement of Luminance Change Depending on Voltage Change

Luminance was measured by using a luminance meter (Minolta Cs-1000A), while the voltage of the organic light emitting diodes was increased from 0 V to 10 V.

### (3) Measurement of Luminous Efficiency

Luminous efficiency (cd/A) at the same current density (10 mA/cm²) were calculated by using the luminance and current density from (1) and (2) above and voltage.

The luminous efficiency values of Examples 1 to 3 and Comparative Examples 1 to 3 were calculated as relative values based on Comparative Example 1 and are shown in Table 1.

### (4) Measurement of Life-Span

Time when each current efficiency (cd/A) was reduced to 97%, while maintaining luminance (cd/m²) at 24,000 cd/m², was measured as a life-span.

The luminous efficiency values of Examples 1 to 3 and Comparative Examples 1 to 3 were calculated as relative values based on Comparative Example 1 and are shown in Table 1.

### (5) Measurement of Driving Voltage

The results were obtained by measuring the driving voltage of each device at 15 mA/cm² using a current-voltage meter (Keithley 2400).

The driving voltages of Examples 1 to 3 and Comparative Examples 1 to 3 were calculated as relative values based on Comparative Example 1 and are listed in Table 1.

TABLE 1

| | | Luminous | Life- | Driving |
|---|---|---|---|---|
| | | efficiency | span | voltage |
| No. | Host | (%) | (%) | (%) |
| Example 1 | A-2/C-4 | 105 | 150 | 103 |
| Example 2 | A-8/C-4 | 107 | 140 | 99 |
| Example 3 | A-9/C-4 | 108 | 160 | 97 |
| Comparative | R-1/C-4 | 100 | 100 | 100 |
| Example 1 | | | | |
| Comparative | R-2/C-4 | 95 | 110 | 97 |
| Example 2 | | | | |
| Comparative | R-3/B-136 | 106 | 40 | 115 |
| Example 3 | | | | |

Referring to Table 1, the organic light emitting diodes according to Examples 1 to 3 exhibited significantly improved luminous efficiency and life-span characteristics compared to the organic light emitting diodes according to Comparative Examples 1 to 3.

By way of summation and review, some example embodiments may provide a composition for an organic optoelectronic device that can lower the driving voltage and realize an organic optoelectronic device with high efficiency and long life-span.

Some example embodiments may provide an organic optoelectronic device including the composition for an organic optoelectronic device.

Some example embodiments may provide a display device including the organic optoelectronic device.

High-efficiency, long life-span organic optoelectronic devices can be realized while lowering the operating voltage.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. A composition for an organic optoelectronic device, the composition comprising:
a first compound represented by Chemical Formula 1; and
a second compound represented by a combination of Chemical Formula 2 and Chemical Formula 3: 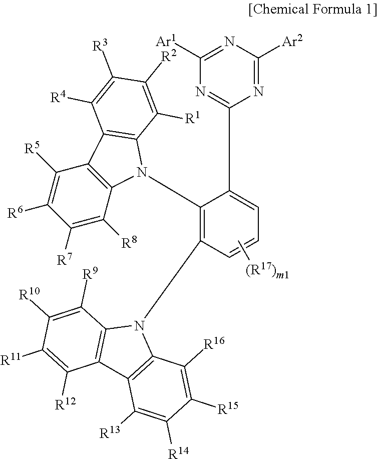
wherein, in Chemical Formula 1,
R¹ to R¹⁷ are each independently hydrogen, deuterium, a cyano group, a substituted or unsubstituted C1 to C10 alkyl group, a substituted or unsubstituted C1 to C10 alkylsilyl group, or a substituted or unsubstituted C6 to C30 aryl group,
at least one of R¹ to R¹⁶ is deuterium, a C1 to C10 alkyl group substituted with one or more deuterium atoms, a C1 to C10 alkylsilyl group substituted with one or more deuterium atoms, or a C6 to C30 aryl group substituted with one or more deuterium atoms,
Ar¹ and Ar² are each independently a substituted or unsubstituted C6 to C30 aryl group or a substituted or unsubstituted C2 to C30 heterocyclic group,
m1 is an integer of 1 to 3, and
when m1 is 2 or 3, each R¹⁷ is the same or different from each other; 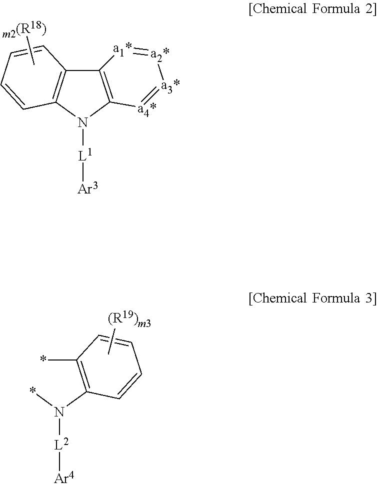
wherein, in Chemical Formula 2 and Chemical Formula 3,
two adjacent ones of a₁* to a₄* of Chemical Formula 2, are linking carbons linked at * of Chemical Formula 3,
the remaining two of a₁* to a₄* of Chemical Formula 2 that are not linked at * of Chemical Formula 3 are C-L^{a}-R^{a},
L^{a}, L¹, and L² are each independently a single bond or a substituted or unsubstituted C6 to C20 arylene group,
R^{a}, R¹⁸, and R¹⁹ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group,
Ar³ and Ar⁴ are each independently a substituted or unsubstituted C6 to C20 aryl group or a substituted or unsubstituted C2 to C30 heterocyclic group,
m2 and m3 are each independently an integer of 1 to 4,
when m2 is 2, 3, or 4, each R¹⁸ is the same or different from each other, and
when m3 is 2, 3, or 4, each R¹⁹ is the same or different from each other.

2. The composition for an organic optoelectronic device as claimed in claim 1, wherein at least one of R¹ to R⁸ is deuterium, a C1 to C10 alkyl group substituted with one or more deuterium atoms, a C1 to C10 alkylsilyl group substituted with one or more deuterium atoms, or a C6 to C30 aryl group substituted with one or more deuterium atoms.

3. The composition for an organic optoelectronic device as claimed in claim 1, wherein R¹ to R⁸ are each independently deuterium, a C1 to C10 alkyl group substituted with one or more deuterium atoms, a C1 to C10 alkylsilyl group substituted with one or more deuterium atoms, or a C6 to C30 aryl group substituted with one or more deuterium atoms.

4. The composition for an organic optoelectronic device as claimed in claim 1, wherein at least one of R⁹ to R¹⁶ is deuterium, a C1 to C10 alkyl group substituted with one or more deuterium atoms, a C1 to C10 alkylsilyl group substituted with one or more deuterium atoms, or a C6 to C30 aryl group substituted with one or more deuterium atoms.

5. The composition for an organic optoelectronic device as claimed in claim 1, wherein R⁹ to R¹⁶ are each independently deuterium, a C1 to C10 alkyl group substituted with one or more deuterium atoms, a C1 to C10 alkylsilyl group substituted with one or more deuterium atoms, or a C6 to C30 aryl group substituted with one or more deuterium atoms.

6. The composition for an organic optoelectronic device as claimed in claim 1, wherein R¹ to R¹⁶ are each independently deuterium, a C1 to C10 alkyl group substituted with one or more deuterium atoms, a C1 to C10 alkylsilyl group substituted with one or more deuterium atoms, or a C6 to C30 aryl group substituted with one or more deuterium atoms.

7. The composition for an organic optoelectronic device as claimed in claim 1, wherein Ar¹ and Ar² are each independently a substituted or unsubstituted phenyl group, a substituted or unsubstituted biphenyl group, a substituted or unsubstituted dibenzofuranyl group, a substituted or unsubstituted dibenzothiophenyl group, or a substituted or unsubstituted carbazolyl group.

8. The composition for an organic optoelectronic device as claimed in claim 1, wherein R¹⁷ is hydrogen, deuterium, a substituted or unsubstituted C1 to C6 alkyl group, a substituted or unsubstituted C1 to C6 alkylsilyl group, or a substituted or unsubstituted C6 to C12 aryl group.

9. The composition for an organic optoelectronic device as claimed in claim 1, wherein the first compound is a compound of Group 1: 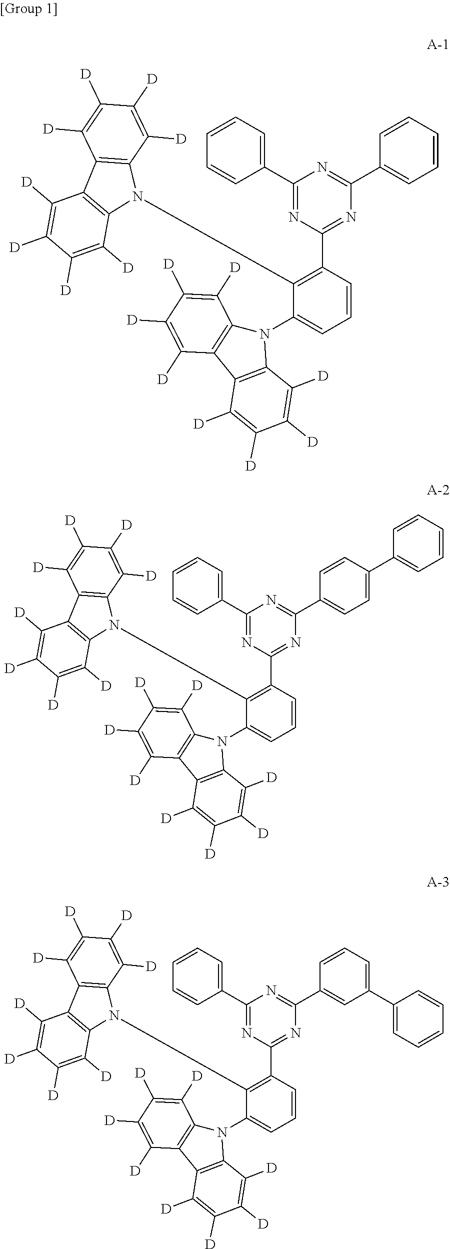 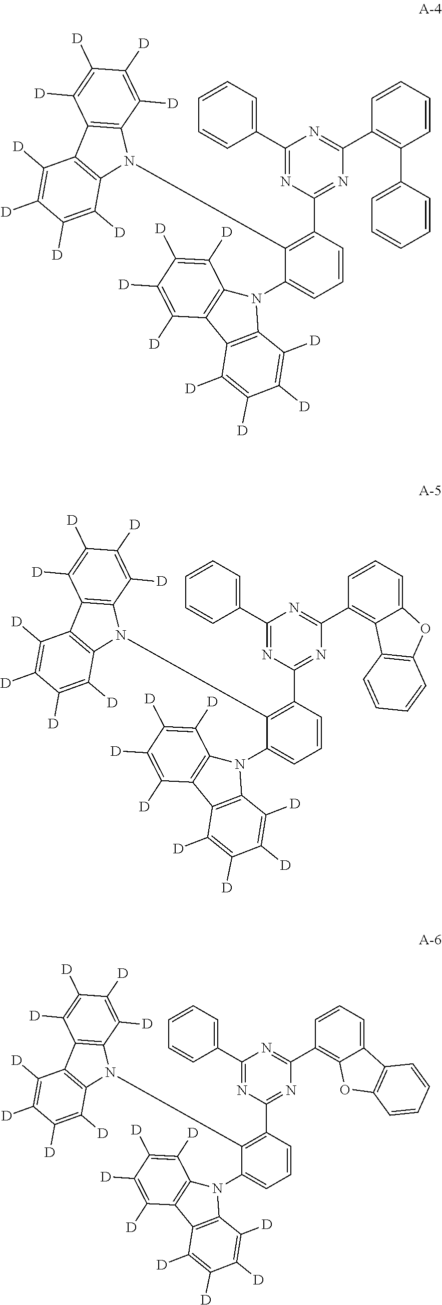 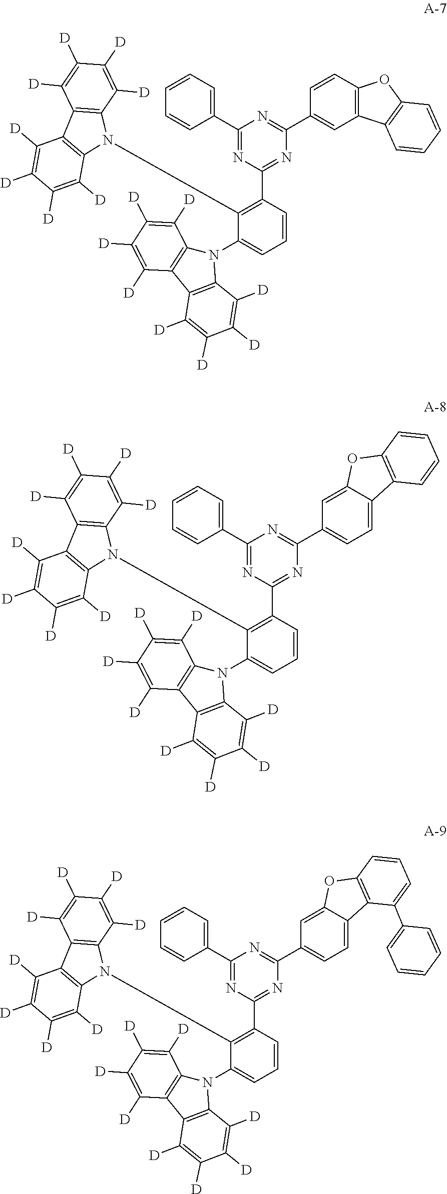 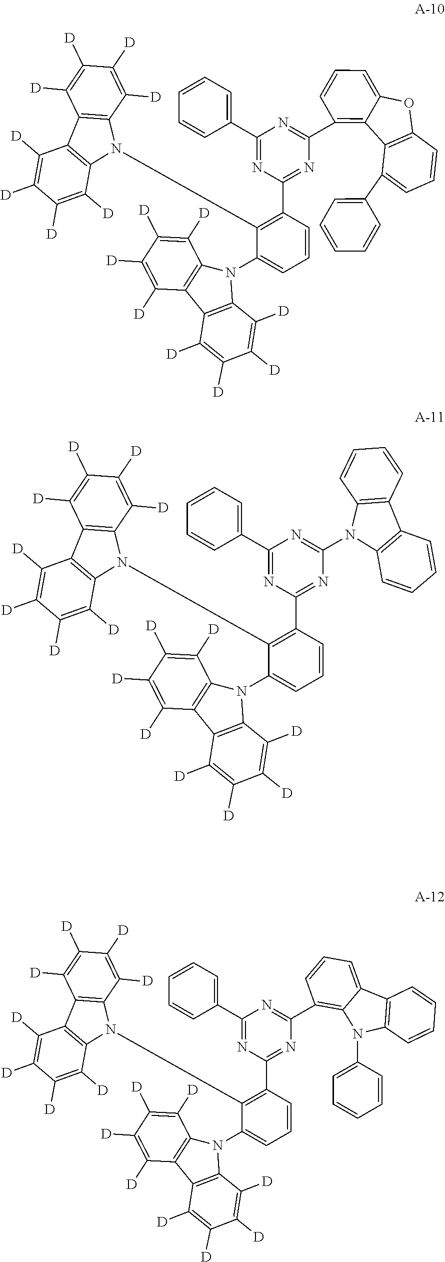 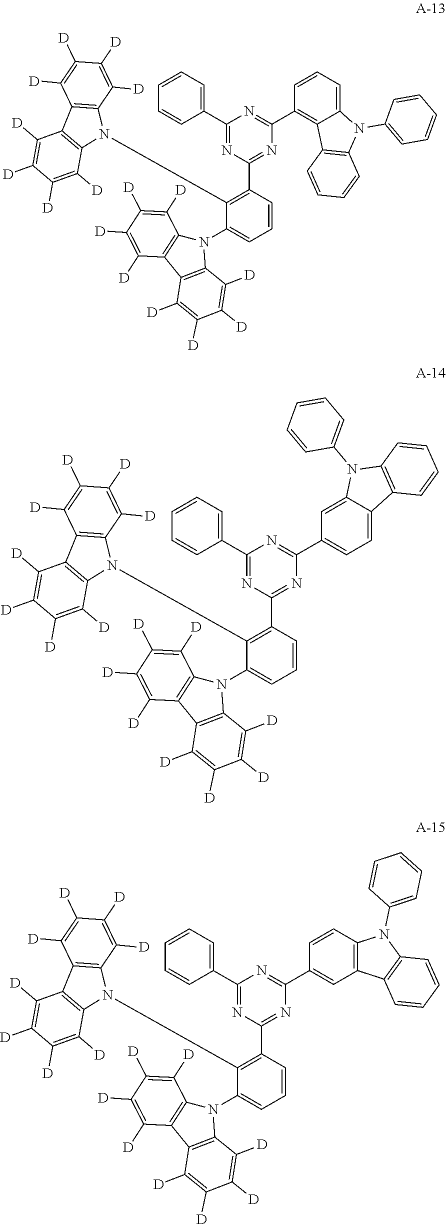 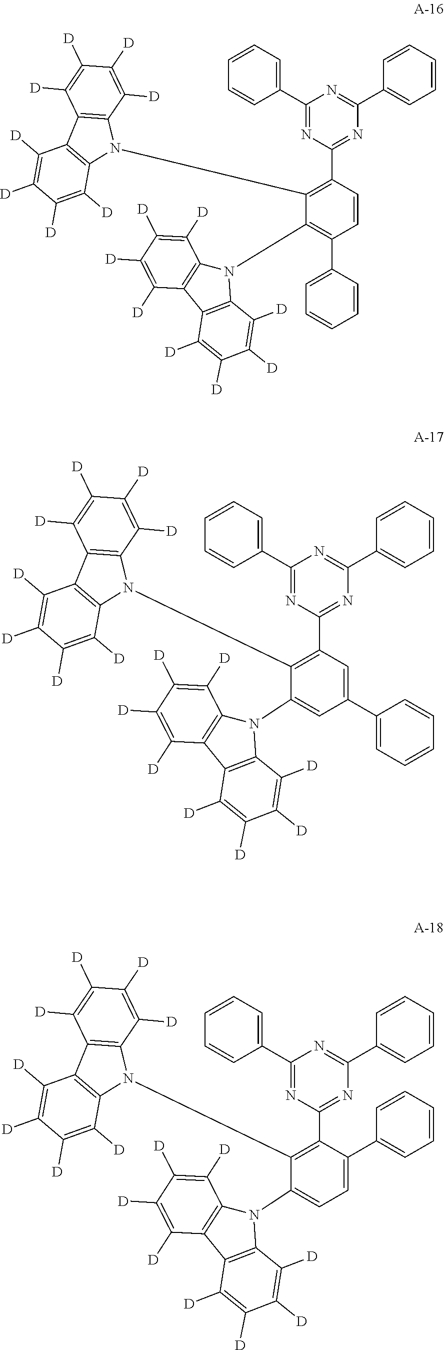 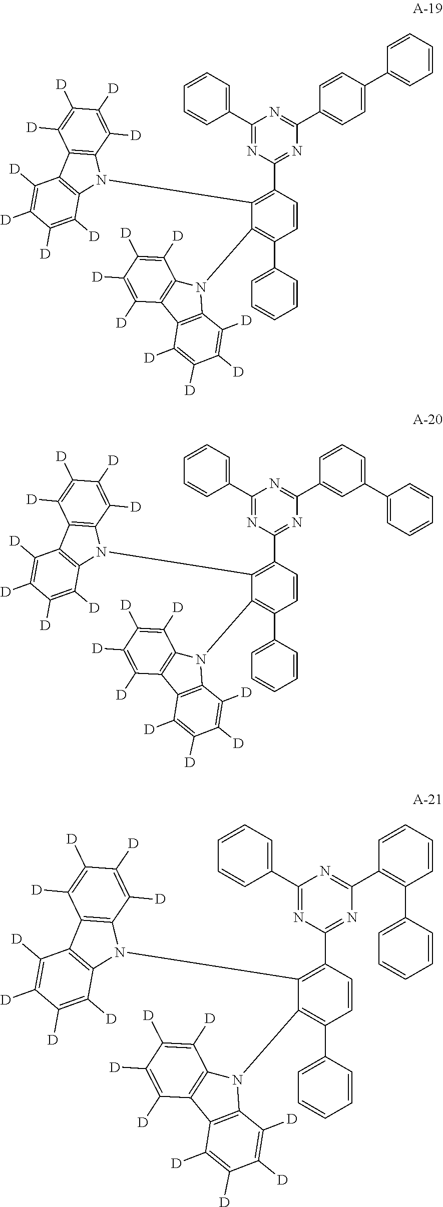 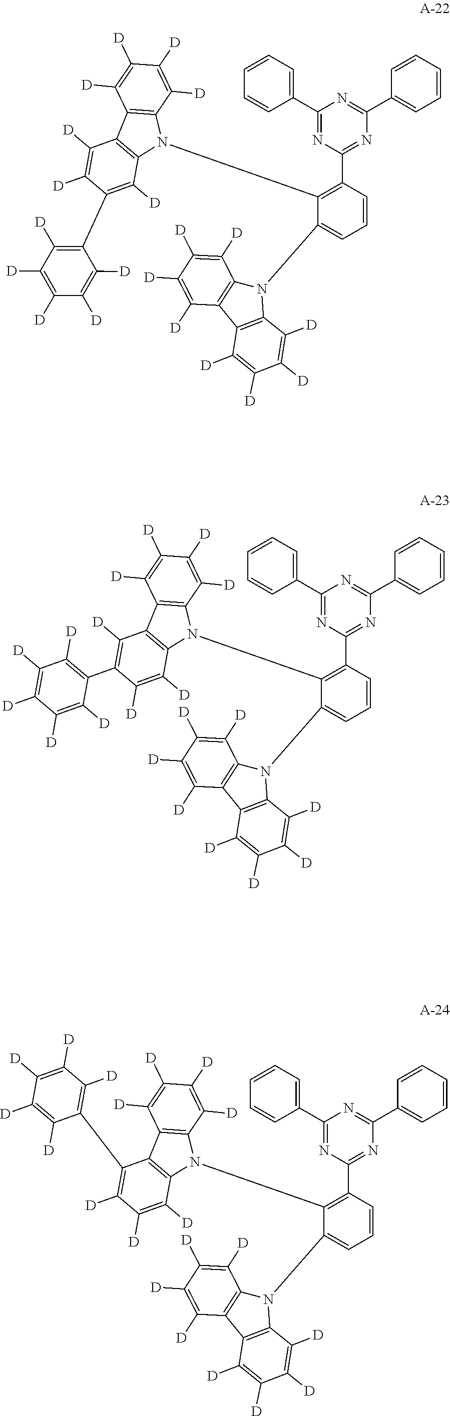 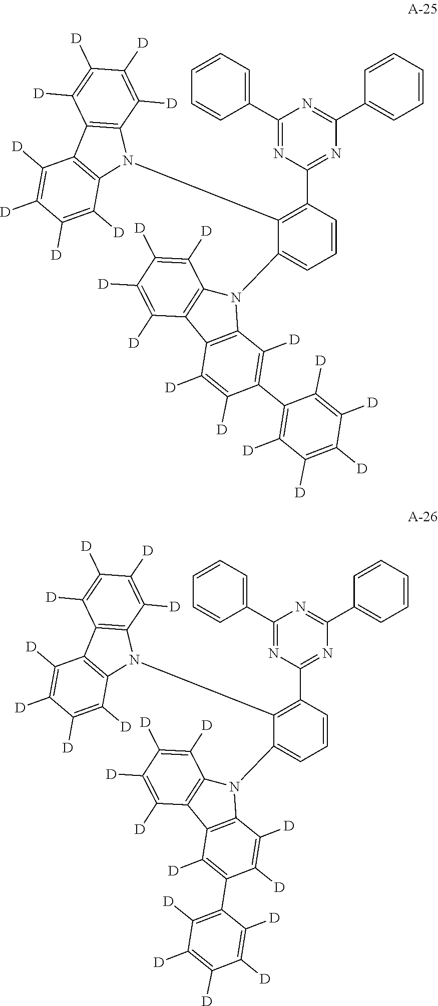 [SMILES: [2H]c1c([2H])c([2H])c(-c2c([2H])c([2H])c3c4c([2H])c([2H])c([2H])c([2H])c4n(-c4cccc(-c5nc(-c6ccccc6)nc(-c6ccccc6)n5)c4-n4c5c([2H])c([2H])c([2H])c([2H])c5c5c([2H])c([2H])c([2H])c([2H])c54)c3c2[2H])c([2H])c1[2H] ; [2H]c1c([2H])c([2H])c(-c2c([2H])c([2H])c3c(c2[2H])c2c([2H])c([2H])c([2H])c([2H])c2n3-c2cccc(-c3nc(-c4ccccc4)nc(-c4ccccc4)n3)c2-n2c3c([2H])c([2H])c([2H])c([2H])c3c3c([2H])c([2H])c([2H])c([2H])c32)c([2H])c1[2H]] 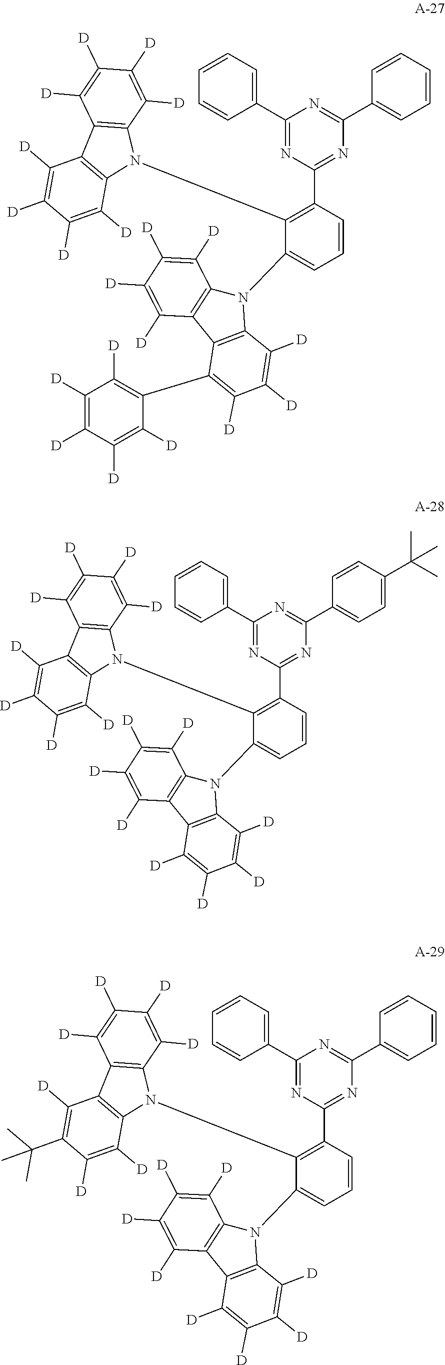 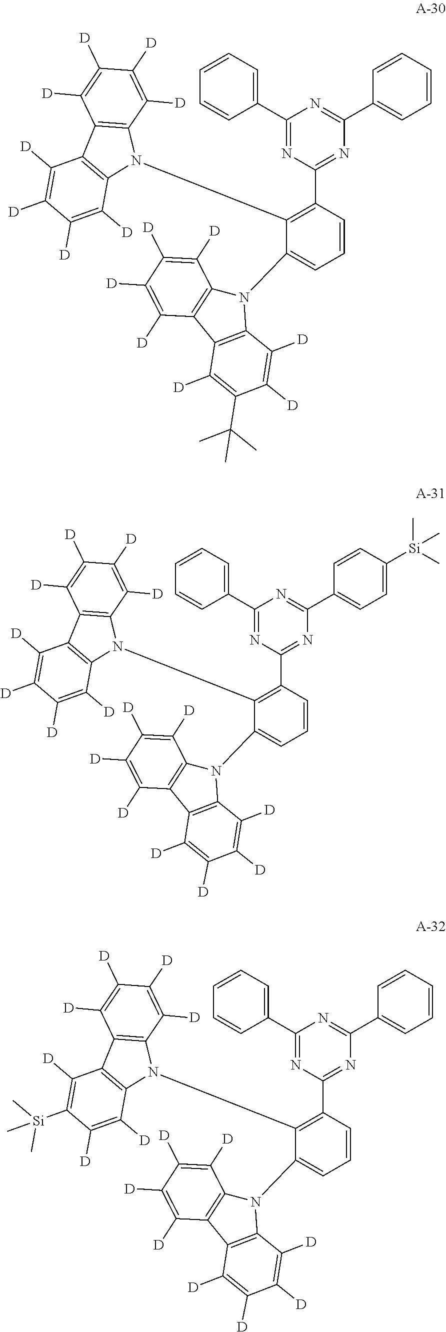 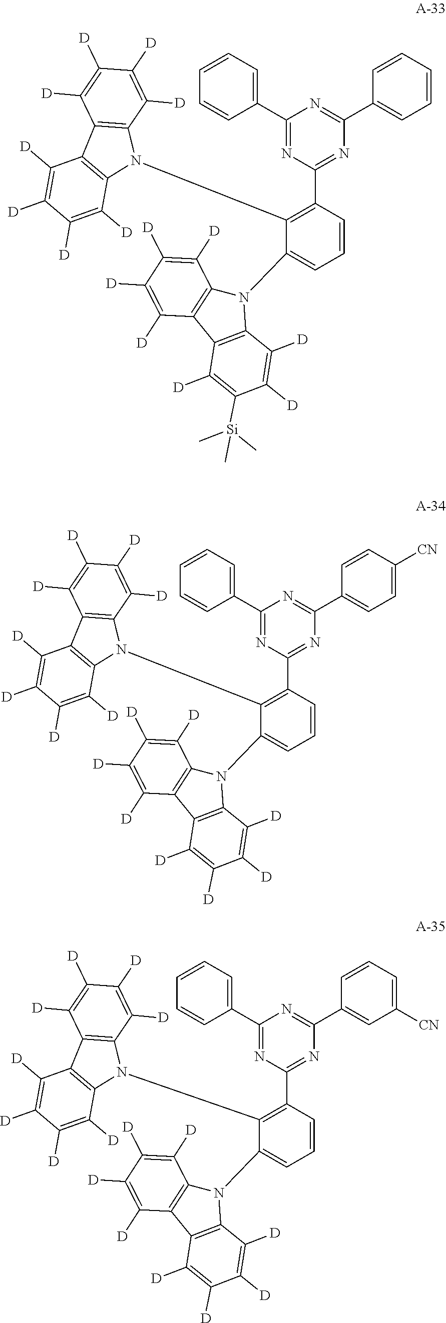 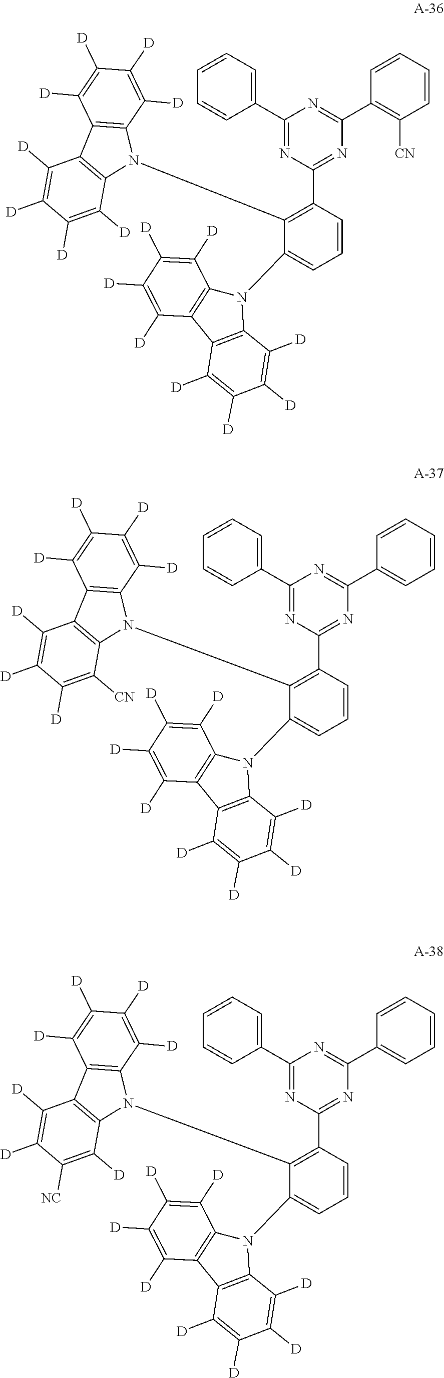 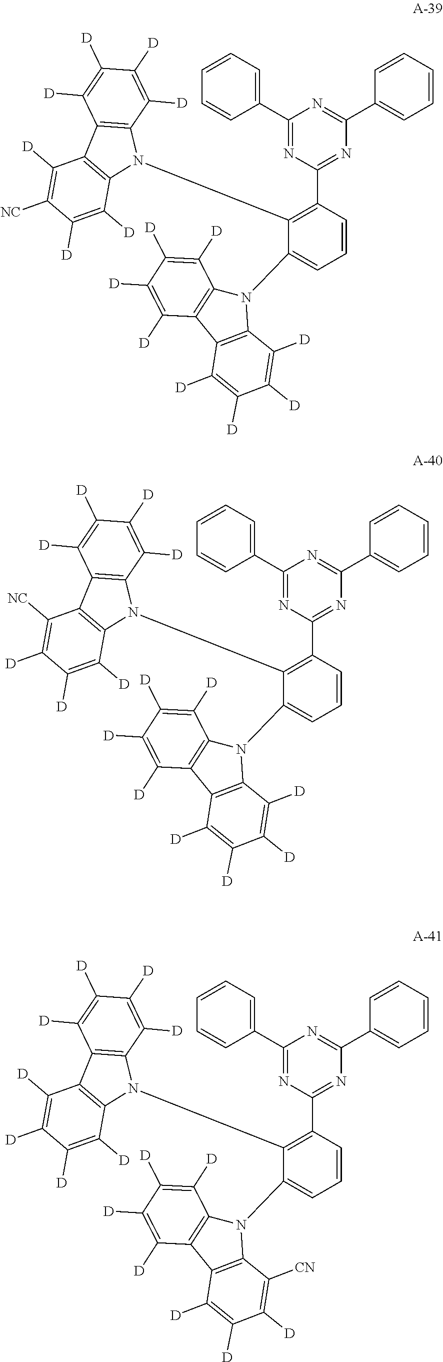 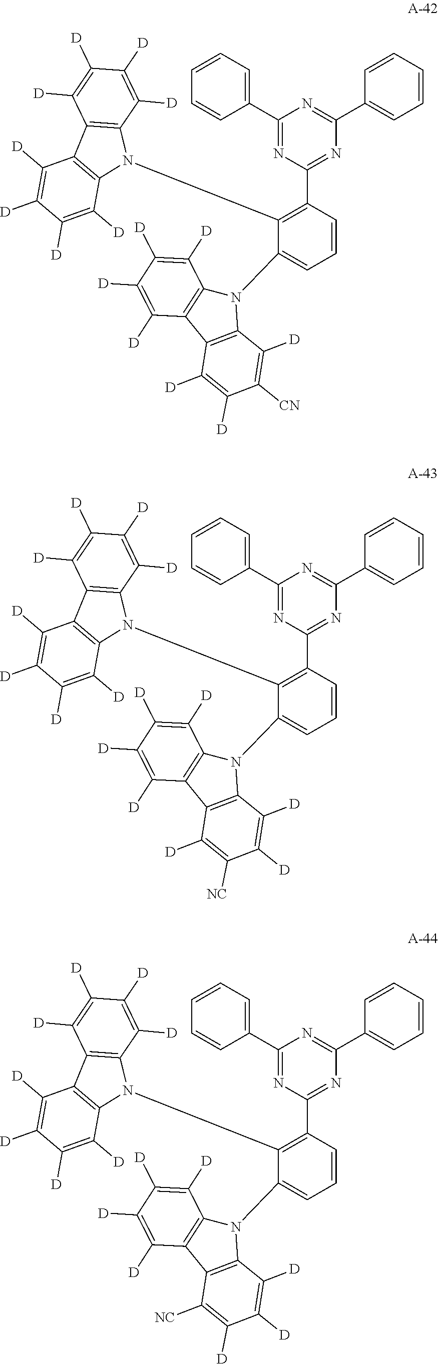 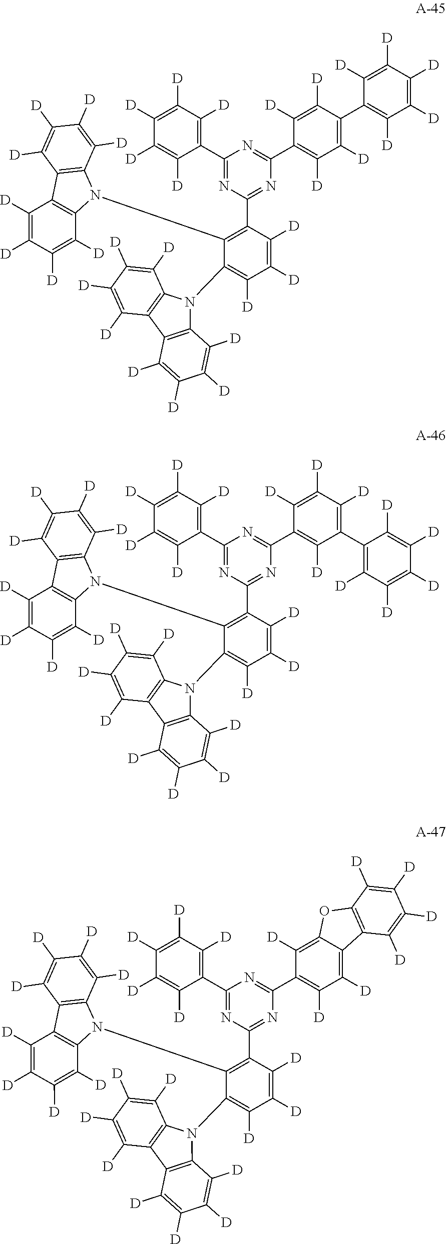 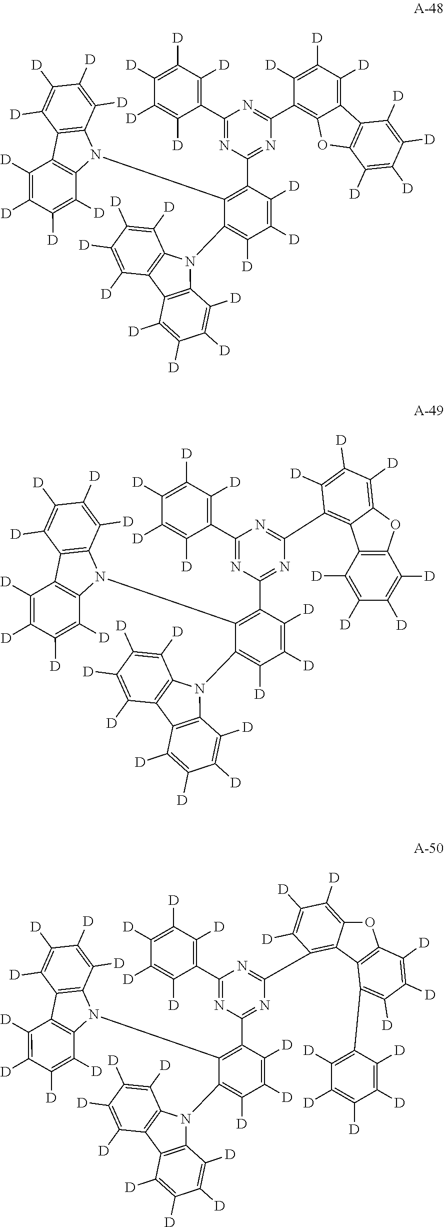
wherein D refers to deuterium.

10. The composition for an organic optoelectronic device as claimed in claim 1, wherein:
the second compound is represented by Chemical Formula 2C: 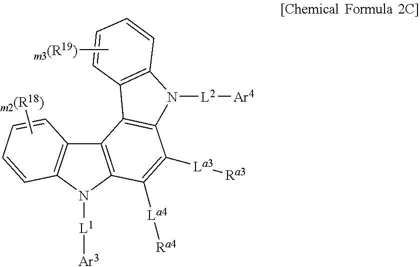
in Chemical Formula 2C,
L^{a3} and L^{a4} are a single bond,
R¹⁸, R¹⁹, R^{a3}, and R^{a4} are each independently hydrogen, deuterium, or a substituted or unsubstituted C6 to C12 aryl group,
m2 and m3 are each independently an integer of 1 to 4, and
moieties *-L¹-Ar³ and *-L²-Ar⁴ are each independently a moiety of Group I, 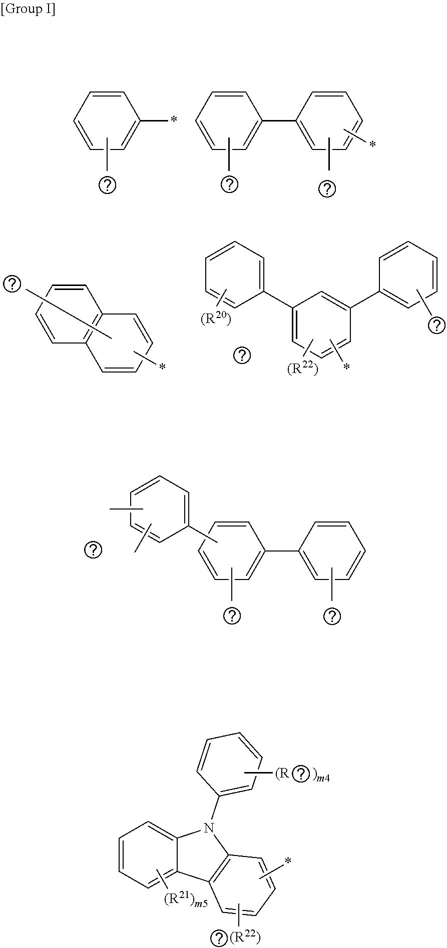 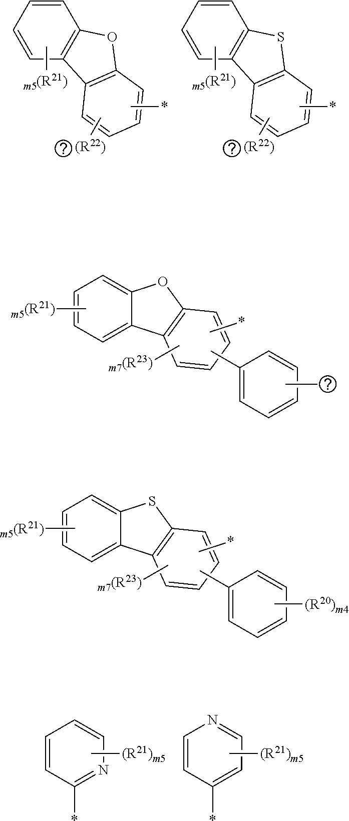 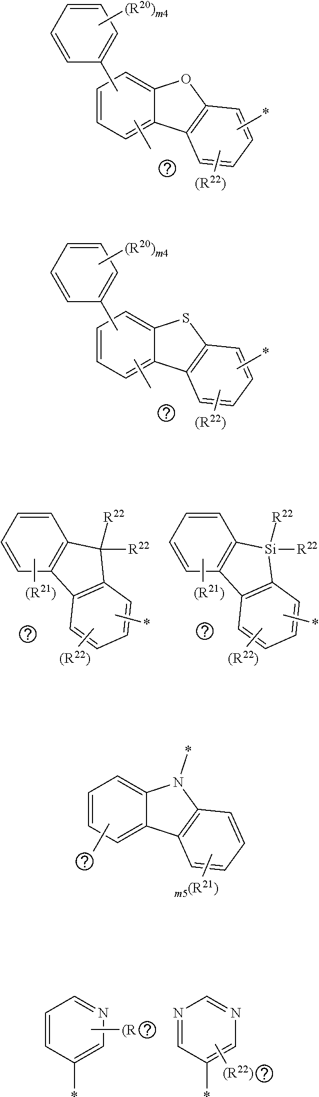 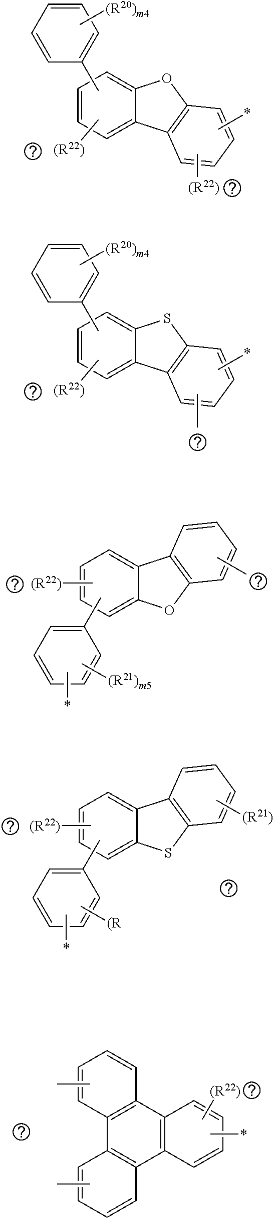 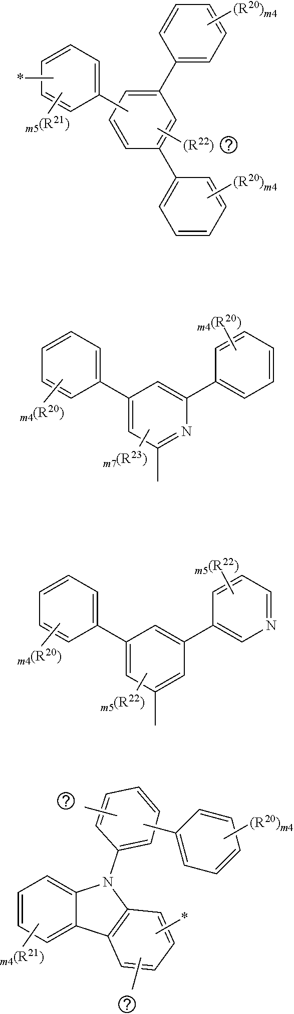 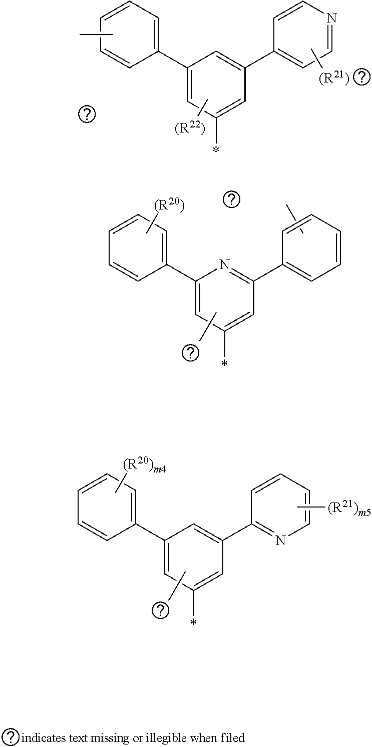
in Group I
R²⁰ to R²⁴ are each independently hydrogen, deuterium, a cyano group, a substituted or unsubstituted C1 to C10 alkyl group, or a substituted or unsubstituted C6 to C12 aryl group,
m4 is an integer of 1 to 5,
m5 is an integer of 1 to 4,
m6 is an integer of 1 to 3,
m7 is an integer of 1 or 2,
m8 is an integer of 1 to 7, and
* is a linking point.

11. An organic optoelectronic device, comprising:
an anode and a cathode facing each other, and
at least one organic layer between the anode and the cathode,
wherein the at least one organic layer includes the composition for the organic optoelectronic device as claimed in claim 1.

12. The organic optoelectronic device as claimed in claim 11, wherein:
the at least one organic layer includes a light emitting layer, and
the light emitting layer includes the composition for an organic optoelectronic device.

13. A display device comprising the organic optoelectronic device as claimed in claim 11.
